(19) United States
(12) Patent Application Publication
(10) Pub. No.: US 2023/0391881 A1
(43) Pub. Date: Dec. 7, 2023

(54) COMPOSITIONS AND METHODS FOR OVERCOMING DR5-INDUCED IMMUNE EVASION BY SOLID TUMORS

(71) Applicant: University of Virginia Patent Foundation, Charlottesville, VA (US)

(72) Inventors: Jogender Tushir-Singh, Davis, CA (US); Sanchita Bhatnagar, Davis, CA (US)

(73) Assignee: University of Virginia Patent Foundation, Charlottesville, VA (US)

(21) Appl. No.: 18/031,969

(22) PCT Filed: Oct. 14, 2021
(86) PCT No.: PCT/US2021/055017

§ 371 Date: Apr. 14, 2023

Related U.S. Application Data
(60) Provisional application No. 63/091,689, filed on Oct. 14, 2020.

(51) Int. Cl. C07K 16/28; A61K 31/5377; A61K 31/4439; A61P 35/00

(52) U.S. Cl. CPC C07K 16/2878; A61K 31/5377; A61K 31/4439; C07K 16/2827; A61P 35/00; A61K 2039/507

(57) ABSTRACT

Provided are methods for treating tumors and/or cancers in subjects in need thereof. In some embodiments, the method include administering to the subject an inhibitor of a Rho-Associated Coiled-Coil Containing Protein Kinase 1 (ROCK1) 5 biological activity and/or a checkpoint inhibitor. In some embodiments, the cancer is a solid tumor. Also provided are compositions that have an effective amount of an inhibitor of a Rho-Associated Coiled-Coil Containing Protein Kinase 1 (ROCK1) biological activity and/or an effective amount of a checkpoint inhibitor; and an effective amount of a DR5 agonist, which can be a bispecific antibody; bispecific antibodies that has a first antigen binding site that is specific for the DRS polypeptide and a second antigen binding site that is specific for the ROCK1 polypeptide, the CTLA4 polypeptide, the PD-1 polypeptide, or the PD-L1 polypeptide; compositions for use in treating tumors and/or cancers, including bispecific antibodies.

### CROSS-REFERENCE TO RELATED APPLICATION

The presently disclosed subject matter claims the benefit of U.S. Provisional Patent Application Ser. No. 63/091,689 filed Oct. 14, 2020, the disclosure of which is incorporated herein by reference in its entirety.

### GRANT STATEMENT

This invention was made with government support under Grant No. CA233752 awarded by the National Institutes of Health and under Grant Nos. W81XWH-18-1-0048,W81XWH-18-1-0049, and W81XWH-19-1-0190 awarded by the Department of Defense. The government has certain rights in the invention.

### REFERENCE TO SEQUENCE LISTING

The Sequence Listing associated with the instant disclosure has been electronically submitted to the United States Patent and Trademark Office as a 157 kilobyte ASCII text file created on Oct. 14, 2021 and entitled “3062_137_PCT_ST25.txt”. The Sequence Listing submitted via EFS-Web is hereby incorporated by reference in its entirety.

### BACKGROUND

In last few decades monoclonal and bispecific antibodies have shown great potential to mobilize immune system against cancer cells (Brinkmann & Kontermann, 2017; Mellman et al., 2011). Unfortunately, despite numerous FDA approvals for blood cancers and melanomas, immunotherapy based strategies have shown significant disappointment in clinical trials of ovarian, glioblastoma, pancreatic, lung and triple negative breast (TNBC) solid tumors. Considering differential architecture of tumor microenvironments, it is a well-accepted fact in the field that limited immune penetration in solid cancer is a major factor for the failure of immunotherapies (Melero, Rouzaut et al., 2014). Therefore, ever since its discovery, one targeting approach called pro-apoptotic receptor agonists (PARA) therapy using Trail ligand (Apo2L) or epithelial cancer enriched death receptor 5 (DR5/TRAIL-R2) activating antibodies (Ashkenazi, 2008) gained significant attention. PARA activate extrinsic apoptotic pathway by oligomerizing DR5, a hallmark of TNF receptor superfamily members (Ashkenazi & Herbst, 2008). In addition, important findings that DR5 can be activated using agonist antibodies to induce cell-death in p53 mutant cancer cells, many DR5 antibodies went to human clinical trials (Ashkenazi & Herbst, 2008; Wu et al., 1997). These DR5 agonist antibodies included lexatumumab (Marini, 2006; Human Genome Sciences, Rockville, Maryland, United States of America), apomab (Camidge, 2008; Genentech Inc., South San Francisco, California, United States of America), AMG655 (Kaplan-Lefko et al., 2010; Amgen Inc., Thousand Oaks, California, United States of America), and Tigatuzumab (Forero-Torres et al., 2010; University of Alabama, Tuscaloosa, Alabama, United States of America), which were highly effective in various immunodeficient xenograft solid tumor models (Camidge, 2008; Kaplan-Lefko et al., 2010; Motoki et al., 2005; Zhang et al., 2007).

None of the DR5 agonist antibodies has been FDA approved due to disappointing overall clinical results (Wajant, 2019). Since then, multiple efforts have been directed to generate second-generation DR5 activators (Tamada et al., 2015; Wajant, 2019). Reports have described DR5 ligand-antibody co-targeting and bispecific antibody based approaches to increase DR5 signaling in tumors (Graves et al., 2014, Shivange et al., 2018, Wajant, 2019). Although these strategies improve DR5 activation, it is well established that a substantial proportion of apoptotic-based therapeutics acquire intrinsic resistance and do not cure cancer (Wajant, 2019). As harnessing immune system has emerged as a powerful tool for oncologic therapeutics, in accordance with the presently disclosed subject matter it is sought to investigate whether factors that temper the potential to orchestrate a complementary immune activation function against solid tumors have contributed to the clinical failure of DR5 agonist antibodies.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to methods for treating tumors and/or cancers in subjects in need thereof. In some embodiments, the methods comprise, consist essentially of, or consist of administering to the subject (a) a composition comprising an effective amount of an inhibitor of a Rho-Associated Coiled-Coil Containing Protein Kinase 1 (ROCK1) biological activity and/or an effective amount of a checkpoint inhibitor, optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor, or any combination thereof, and (b) a composition comprising an effective amount of a Death Receptor 5 (DR5) agonist. In some embodiments, the cancer comprises a solid tumor, optionally a solid tumor selected from the group consisting of an ovarian tumor, a glioblastoma, a pancreatic tumor, a lung tumor, and a triple negative breast (TNBC) tumor. In some embodiments, the inhibitor of ROCK1 activity is a small molecule inhibitor. In some embodiments, the inhibitor of ROCK1 activity is selected from the group comprising N-(3-{[2-(4-Amino-1,2,5-oxadiazol-3-yl)-1-ethyl-1H-imidazo[4,5-c]pyridin-6-yl]oxy}phenyl)-4-[2-(morpholin-4-yl)ethoxy]benzamide (GSK269) and N-(6-Fluoro-1H-indazol-5-yl)-6-methyl-2-oxo-4-[4-(trifluoromethyl)phenyl]-3,4-dihydro-1H-pyridine-5-carboxamide (GSK429). In some embodiments, the checkpoint inhibitor comprises an antibody, optionally an antibody that binds to a CTLA4 polypeptide, a PD-1 polypeptide, and/or a PD-L1 polypeptide. In some embodiments, the antibody is selected from the group consisting of avelumab, atezolizumab, durvalumab, nivolumab, pembrolizumab, spartalizumab, tremelimumab, and ipilimumab. In some embodiments, the DR5 agonist comprises a DR5 targeting antibody. In some embodiments, the DR5 targeting antibody is selected from the group comprising lexatumumab, apomab, AMG655, LBy135, WD-1, KMTR2, and tigatuzumab. In some embodiments, the composition comprises (a) an effective amount of an inhibitor of a ROCK1 biological activity and/or an effective amount of a checkpoint inhibitor, optionally wherein the checkpoint inhibitor is a CTLA4 inhibitor, a PD-1 inhibitor, a PD-L1 inhibitor, or any combination thereof, and (b) an effective amount of a DR5 agonist in a single composition.

The presently disclosed subject matter also relates in some embodiments to compositions comprising, consisting essentially of, or consisting of (a) an effective amount of an inhibitor of a Rho-Associated Coiled-Coil Containing Protein Kinase 1 (ROCK1) biological activity and/or an effective amount of a checkpoint inhibitor, optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor, or any combination thereof, and (b) an effective amount of a DR5 agonist. In some embodiments, the composition comprises a bispecific antibody. In some embodiments, the composition further comprises, consists essentially of, or consists of a pharmaceutically acceptable carrier, optionally a pharmaceutically acceptable carrier that is pharmaceutically acceptable for use in a human.

The presently disclosed subject matter also relates in some embodiments to bispecific antibodies that binds to a death receptor 5 (DR5) polypeptide and second polypeptide selected from the group consisting of a Rho-Associated Coiled-Coil Containing Protein Kinase 1 (ROCK1) polypeptide, a Cytotoxic T-Lymphocyte Associated Protein 4 (CTLA4) polypeptide, a Programmed Cell Death Protein 1 (PD-1) polypeptide, and a Programmed Death-Ligand 1 (PD-L1) polypeptide, wherein said bispecific antibody comprises a first antigen binding site that is specific for the DR5 polypeptide and a second antigen binding site that is specific for the ROCK1 polypeptide, the CTLA4 polypeptide, the PD-1 polypeptide, or the PD-L1 polypeptide. In some embodiments, the bispecific antibody comprises a heavy chain variable region and/or a light chain variable as set forth in any of SEQ ID NOs: 1-12. In some embodiments, the bispecific antibody is humanized. In some embodiments, a bispecific antibody of the presently disclosed subject matter further comprises, consists essentially of, or consists of a pharmaceutically acceptable carrier, optionally a pharmaceutically acceptable carrier that is pharmaceutically acceptable for use in a human.

The presently disclosed subject matter also relates in some embodiments to compositions for use in treating tumors and/or cancers. In some embodiments, the compositions comprise, consist essentially of, or consist of (a) an effective amount of an inhibitor of a Rho-Associated Coiled-Coil Containing Protein Kinase 1 (ROCK1) biological activity and/or an effective amount of a checkpoint inhibitor, optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor, or any combination thereof, and (b) an effective amount of a Death Receptor 5 (DR5) agonist. In some embodiments, the composition comprises a bispecific antibody, and further wherein the bispecific antibody comprises a first antigen binding site that is specific for the DR5 polypeptide and a second antigen binding site that is specific for a ROCK1 polypeptide, a CTLA4 polypeptide, a PD-1 polypeptide, and/or a PD-L1 polypeptide. In some embodiments, a composition for use of the presently disclosed subject matter further comprises, consists essentially of, or consists of a pharmaceutically acceptable carrier, optionally a pharmaceutically acceptable carrier that is pharmaceutically acceptable for use in a human.

The presently disclosed subject matter also relates in some embodiments to bispecific antibodies for use in treating tumors and/or cancers. In some embodiments, a bispecific antibody of the presently disclosed subject matter comprises, consist essentially of, or consists of a first binding activity that binds to a death receptor 5 (DR5) polypeptide and a second binding activity that binds to a ROCK1 polypeptide, a CTLA4 polypeptide, a PD-1 polypeptide, and/or a PD-L1 polypeptide. In some embodiments, a bispecific antibody of the presently disclosed subject matter comprises a heavy chain variable region and/or a light chain variable as set forth in any of SEQ ID NOs: 1-12. In some embodiments, a bispecific antibody is humanized. In some embodiments, a bispecific antibody for use in treating a tumor and/or a cancer of the presently disclosed subject matter further comprises, consists essentially of, or consists of a pharmaceutically acceptable carrier, optionally a pharmaceutically acceptable carrier that is pharmaceutically acceptable for use in a human.

Accordingly, it is an object of the presently disclosed subject matter to provide compositions and methods for treating tumors and/or cancers. This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, Figures, and Examples.

### BRIEF DESCRIPTIONS OF THE FIGURES

FIGS. 1A-1M. DR5 agonist antibodies surface stabilizes PD-L1 on solid cancer cells and tumors. (FIGS. 1A, 1B, and 1D) Total PD-L1 and PARP from colon (Colo-205), lung (A549), pancreatic (PANK1), triple negative breast cancer cell (MDA-MB-436), and ovarian cell (Cavo-3) lysates treated with indicated DR5 agonist antibodies named Lexa (lexatumumab), KMTR2, BaCa, and tigatuzumab. GAPDH is loading control. (FIG. IC) Total PD-L1, CD47, Calreticulin, PARP from MDA-MB-436 cell lysates treated with indicated DR5 agonist antibodies ±caspase inhibitor Z-VAD. GAPDH is loading control. (FIG. 1E) Total PD-L1 western blotting signal from the lysates of OVCAR-3 (n=3) and MDA-MB-436 (n=3) cells after 6 hours treatment of 100 nM lexa (DR5 agonist antibody) was normalized to GAPDH. Representative blots are in FIG. 20A. (FIG. 1F) Surface biotinylation of PD-L1 from indicated tumor cells after indicated DR5 agonist treatments. In lane 3 and 4, KMTR2 was pre-neutralized either with recombinant DR5 (rDR5) or recombinant FOLR1 (rFOLR1) (FIG. 1G) Representative flow cytometry plots of PD-L1 from two different tumor cell lines treated with indicated DR5 antibodies. Secondary alone and IgG1 control are included for background. (FIG. 1H) Relative surface PD-L1% cells, after DR5 agonist treatments (see FIGS. 8B, 19A, and 19B). Relative signal is normalized to surface PD-L1 in IgG1 treated cells in corresponding tumor cells (n=3-4) (FIG. 1I) 0.5×10⁶-2×10⁶ indicated tumor cells were injected subcutaneously in NOD.Cg-Prkdc^{scid}I12rg^{tm}1Wj1/SzJ animals with Matrigel in PBS. When tumors appeared on animals (3-4 weeks), animals were i.p. injected with indicated DR5 agonists (4-6 doses), followed by tumor extraction and single cell suspension isolation from tumors after indicated antibody treatments. (FIG. 1J) Relative surface PD-L1% cells in indicated tumors after DR5 agonist treatments (see FIGS. 21A-21C). Relative signal is normalized to surface PD-L1 in IgG1 treated tumors (n=2-6). (FIG. 1K) ER (−), PR (−) and HER2 (−) UCD52 patient-derived tissue was xenografted in breast fat pad of NOD.CgPrkdc^{scid}I12rg^{tm1Wj1}/SzJ mice, following by treatment with IgG1 or KMTR2 (50 μg, 4 doses). Harvested tumors were analyzed for PDL1, CD47 and PARP in lysates. (FIG. 1L) KMTR2 treated (50 μg, 4 doses) UCD52 TNBC PDX tumors were stained for PD-L1 using immunohistochemistry (IHC). For additional images see FIG. 20C. (FIG. 1M) Total PD-L1 and CD47 blotting analysis from DR5 resistant MDA-MB-436 cell lines after indicated DR5 agonist treatment (50 nM, 6 hr). For DR5 resistant cell generation and additional data see FIG. 9A-9C. Data information: Error bars represent SD. In FIG. 1E, FIG. 1H, and FIG. 1J unpaired Welch's t-test was used to determine p values. (*p<0.05, **p<0.005, ***p <0.0001).

FIGS. 2A-2P. PD-L1 stabilization is CSN5 independent but protease dependent. (FIG. 2A) Model of CD3 activation that induces luciferase in PD-1 effector jurkat reporter cells. See also FIGS. 10A-10D. (FIG. 2B) Tumor cell-Jurkat cell co-culture model: Upon DR5 activation in tumor cells, surface mobilized PD-L1 engages PD-1 on jurkat reporter cells leading to loss of luciferase activity. See also FIGS. 10A-10D. (FIG. 2C) Luciferase activity of reporter lines from tumor cell-Jurkat cell co-culture assay using MDA-MB-436 and OVCAR3 cells after treatment (50 nM) with indicated DR5 agonists (tiga: tigatuzumab, AMG: AMG655, KMTR1, Lexa: Lexatumumab). The background luciferase signal from untreated cells (due to basal surface PD-L1) was subtracted. Various controls treatments (IgG1, anti-PD-L1, anti-EGFR) are also shown. (FIG. 2D) Same as FIG. 2C except tumor cells were pre-treated with indicated inhibitors for AKT, ERK, mTOR, MEK, STAT3 NF-Kβ along with DR5 agonist KMTR2 antibody prior to coculture. (FIGS. 2E-2G) MDA-MB-436 cells were treated with TNFα and indicated DR5 agonists for indicated times. Lysates were analyzed for PD-L1, CSN5, phosphorylated p65, total p65, cleaved caspase-3 and PARP. GAPDH is loading control. Additional data from OVCAR-3 is shown in FIG. 11C. (FIG. 2H) Flow cytometry analysis of MDA-MB-436 cells treated with TNFα±MG132 (top) and indicated DR5 agonist±MG132 (bottom). (FIG. 2I) Total PD-L1 from OVCAR3 cell lysates was analyzed after treatment of indicated DR5 agonist ±MG132. Additional data from MDA-MB-436 is shown in FIG. 11F. (FIG. 2J) Flow cytometry surface PD-L1 analysis of OVCAR-3 cells treated with indicated DR5 agonist ±MG132. Flow cytometry data is shown in FIG. 11D. (FIG. 2K) MDA-MB-436 cells were treated with MG132 for indicated times and total PD-L1 from lysates was analyzed. GAPDH is loading control. (FIG. 2L) Immunoblotting of DR5 confirming generation of knock out (DR5-KO) cell lines. (FIG. 2M) Cell viability analysis of DR5-KO and DR5-WT cells. (FIG. 2N) DR5-KO and WT cells were treated with TNFα and indicated DR5 agonist followed by flow cytometry using PD-L1 specific antibodies. See also FIG. 11E. (FIG. 2O) TNBC WT and DR5-KO (MDA-MB-436) cells were treated with either DR5 agonist or TNFα as indicated. Lysates were analyzed for PD-L1, S5a/PSMD4 and DR5. GAPDH is loading control. (FIG. 2P) OVCAR-3 and MDA-MB-436 (WT and DR5-KO) cells were treated with KMTR2 followed by ubiquitin and S5a immunoblotting from total lysates. GAPDH is loading control Data information: Error bars in FIG. 2C and FIG. 2D represent SD. Unpaired Welch's t-test was used to determine p values for FIG. 2C and FIG. 2D. Paired t test was used for M. (*p<0.05, **p <0.005, ***p<0.0001).

FIGS. 3A-3M. ApoEVs and non-apoptotic caspase-8 help stabilize PD-L1 in DR5 insensitive tumor cells. (FIG. 3A) Schematic showing presence of tumor cells capable of optimal and non-optimal apoptotic activation by DR5 agonists constitute heterogeneous tumors. (FIGS. 3B-3D) ApoEVs isolated after IgG1 and KMTR2 treatment (OVCAR-3) were blotted against CD63 and PD-L1 in dot blots. ApoEVs isolated after IgG1 and tigatuzumab (FIG. 3C) and lexatumumab (FIG. 3D) treatment (MDA-MB-436) were run on western and blotted against CD63 and PD-L1. (FIG. 3E) Details of experimental data described in FIG. 3F. (FIG. 3F) ApoEVs isolated from DR5 sensitive tumor cells grown in Met−/HPG+ media were added on to DR5-KO cells (growing in regular media). After 48 hours, flow cytometry analysis was carried out with HPG catalyzing dye. HPG incorporation (3rd plot; see Materials and Methods for the EXAMPLES below for more details) from flow cytometry data confirms ApoEVs transfer from cells growing in Met−/HPG+(DR5-WT) to DR5-KO cells. (FIG. 3G) ApoEVs isolated from DR5-WT cells were added on to DR5-KO (MDA-MB-231) cells for indicated times (24-96 hours) to analyze PD-L1 transfer kinetics from ApoEVs. In top panel ApoEVs were isolated from OVCAR-3 cells and in bottoM panel ApoEVs were isolated from MDA-MB-436. (FIG. 3H) Same as FIG. 3G except after addition of MDA-MB-436 derived ApoEVs (treated with combination of KMTR2+lexatumumab DR5 agonists or IgG1) on to DR5-KO MDA-MB-231 cells, total lysates were immunoblotted for PD-L1 after 24 hours. KMTR2+lexatumumab (n=3), IgG1 (n=2). (FIG. 3I) Left: PD-L1 surface histogram shows direct antibody treatment on DR5-KO cells. Right: PD-L1 surface histogram from DR5-KO cells after treatment (24 hours) with ApoEVs isolated from DR5 sensitive cells after either Lexa or KMTR2 treatment. (FIG. 3J) Same as FIG. 3H and FIG. 3I except ApoEVs treated tumor cells were analyzed in PD-1 reporter co-culture assays for luciferase signal (see FIGS. 2A-2P). (FIG. 3K) MDA-MB-436 cells were treated with KMTR2 for 6 hours and along with ZDEVD for indicated times.−ZDEVD shows final time of KMTR2 exposure to cells in absence inhibitor. Lysates were immunoblotted for cleaved caspase-3, 8, PARP, PD-L1, S5a, ROCK1 and GAPDH. u.c: uncleaved, c: cleaved, n.s: non-specific band (FIG. 3L) Relative caspase-8 and caspase-3 activity assays from FIG. 3K. Similar to cleaved caspase-8 profile in FIG. 3K, caspase-8 maintained steady activity, while caspase-3 activity required at least 40+ mins of DR5 agonist treatment without ZDEVD. (FIG. 3M) After indicated treatments with KMTR2 (DR5 agonist) and ZDEVD (caspase-3 preferred inhibitor) similar to FIG. 3K and FIG. 3L, cells were allowed to grow 24 hours, followed by cell viability analysis. Data information: Error bars in FIG. 3I represent SD. Error bars in FIG. 3L and FIG. 3M represent SEM. Unaired t test was used for FIG. 3J, FIG. 3L, and FIG. 3M. (*p<0.05, **p<0.005, ***p<0.0001).

FIGS. 4A-4K. DR5 agonist activated ROCK1 functions to help PD-L1 surface mobilization. (FIGS. 4A and 4B) KMTR2 and lexatumumab treated MDA-MB-436 and OVCAR3 (respectively) lysates for indicated early time points were analyzed for caspase-8, caspase-3, ROCK1, pMLC, PARP, PD-L1 and CMTM6 etc. as indicated. Vertical arrows indicate sequential kinetics of caspase-8, ROCK1, and caspase-3 activation, arrows with “n.s.” below them indicate nonspecific band by ROCK1 antibody. (FIG. 4C) OVCAR3 cells were treated with pMLC activating ionophore A23187 (positive control) and lexatumumab ±ROCK1 inhibitors or ±rDR5 or ±rFOLR1. Lysates were later analyzed for ROCK1, pMLC, caspase-3 and PARP with GAPDH as loading control. (FIG. 4D) MDA-MB-436 and OVCAR3 cells were treated with indicated ROCK1 inhibitors 2 hours prior to DR5 agonist KMTR2 treatment. After 4 hours flow cytometry was used to analyze surface PD-L1. (GSK269: GSK269962A, GSK429: GSK429286A). (FIG. 4E) MDA-MB-436 cell survival assay after treatment with DR5 agonist antibody KMTR2±ROCK1 inhibitors. (GSK269: GSK269962A, GSK429: GSK429286A) (FIG. 4F) Schematic of immunoprecipitation assay shown in FIG. 4G and FIG. 4H. Cultured MDA-MB-436 cells were treated with KMTR2 (IgG1-Fc) or IgG1 control ±ROCK1i (GSK269962) for 6 hours. After 6 hours, 500 nM anti-PD-L1 avelumab (IgG4-Fc) was added to the media for additional 1 hr. Cellular lysates were pulled down with anti-hu-IgG4 specific beads. (FIG. 4G) Immunoprecipitated lysates and leftover supernatant after various treatments as described in FIG. 4F were run at the same time followed by blotting with ROCK1, PD-L1 and CMTM6. Total lysates as a control were also generated in exactly similar conditions and run next to supernatant and IP samples. (FIG. 4H) Immunoprecipitation assays with anti-PD-L1 (avelumab) same as FIG. 4G using OVCAR-3 cell lines. (FIG. 4I) After treatment with IgG1 control and indicated DR5 agonists (KMTR2, Lexa, Tigatuzumab) ±ROCK1 inhibitors (GSK269962A, GSK429286A) tumor cells were cocultured with anti-CD3 stimulated PD1+ effector Jurkat cells (stably expressing luciferase under NFAT-RE promoter). Relative luciferase signal was quantified and plotted after subtraction of background signal from untreated cells. (FIG. 4J) Relative luciferase activity from assay as described in FIG. 4I. Data is from 4 different DR5 agonist antibodies used in combination of two different ROCK1i inhibitors. (FIG. 4K) Tumor regression efficacy of MD5-1 (50 μg), GSK269962A (<2 mg/kg) and MD5-1+ GSK269962A (50 μg+2 mg/kg) 4T1 tumors (n=4-7). 6-8 weeks old BALB/c mice bearing 4T1 tumors (˜100 mm³) were intraperitoneally (i.p.) injected with 50 μg of indicated antibody every third day (n=4-6). GSK269962A (ROCK1i) was injected directly into the tumors. Tumor volumes were quantified at indicated days by caliper measurements. Data information: Error bars in FIG. 4J represent SD. Statistical significance in FIG. 4J was determined by unpaired student t-test and in FIG. 4K using two-tailed paired Wilcoxon Mann-Whitney test. (*p<0.05, **p<0.005, ***p<0.0001).

FIGS. 5A-5Q. Generation and testing of chimeric DR5 for co-targeting of human DR5 agonist and ROCK1 in immunocompetent murine tumors. (FIG. 5A) Schematic and genetic construction of two chimeric human-mouse DR5 (Chi-DR5, Chi-G4S-DR5) with human extracellular domain and mouse transmembrane (TM) and intracellular domains (ICD). (FIGS. 5B-5D) FACs plots confirming expression of human DR5 (huDR5) and Chi-G4S-DR5 in mouse 4T1 cells. Chi-DR5 was not expressed on cell surface (FIG. 5C). See also FIG. 15A. (FIG. 5E) Cell viability analysis of huDR5 and Chi-G4S-DR5 stable 6T1 cells with indicated human DR5 agonists lexatumumab and KMTR2. (FIG. 5F) Comparison of tumor growth of grafted huDR5 and Chi-G4S-DR5 stable 6T1 cells in Balb/c mice (n=3). (FIG. 5G) Chi-G4S-DR5 stable 6T1 tumors (after reaching ˜300 mm³) were treated with 4 doses (100 g) of IgG1, lexatumumab, lexatumumab +avelumab followed by tumor recovery and surface PD-L1 analysis using flow cytometry similar to described in FIGS. 1I and 1J. See also FIG. 23. (FIG. 5H) Chi-G4S-DR5 sTable 6T1 tumors (after reaching ˜100 mm³) were treated with 6 doses of indicated treatment. Isolated tumors were imaged together (n=4-5). ROCK1i indicates GSK269962A and was administered to animals via intra-tumor injections (2 mg/kg). (FIG. 5I) Same as FIG. 5H, except average tumor weight is shown. (FIGS. 5J and 5K) Confirmation of selective CD8+ T cell population depletion in mice spleen and grafted tumors after injecting animals with anti-CD8a antibody. CD4+ T-cells remained unchanged. (FIG. 5L) Average of tumor weights (harvested at same time) from mice treated (i.p.) with IgG1, lexatumumab, ROCK1i, lexatumumab+ROCK1i and anti-CD8+lexatumumab+ROCK1i (n=4-7, 50 g Lexatumumab, 2 mg/kg ROCK1i, 6 doses). ROCK1i indicates GSK269962A and was administered to animals via intra-tumor injections. Various treatments were started when tumors were ˜100 mm³ size. Also see FIG. 15B (FIG. 5M) Average of tumor weights (harvested at same time) from mice treated (i.p.) with IgG1, lexatumumab, avelumab, lexatumumab+avelumab and anti-CD8+lexatumumab+avelumab (n=4-7). See also FIGS. 15C and 15D for additional confirmation with PD-1 inhibitor. (FIG. 5N) 6-8 weeks old C57BL/6 bearing Chi-G4S-DR5 expressing MC38 tumors were intraperitoneally (i.p.) injected with 50 g of indicated antibody every third day (n=4-6). Various treatments were started when tumors were ˜100 mm³ size. Tumor volumes were quantified at indicated days by caliper measurements. (FIG. 5O) Same as FIG. 5H, except KMTR2 (8 doses) antibody instead of lexatumumab was used as the DR5 agonist for the experiment. (FIG. 5P) Average of tumor weights of data shown in FIG. 5O. (FIG. 5Q) Kaplan-Meier plot depicting the survival of syngeneic Chi-G4S-DR5 4T1 tumor bearing animals injected i.p. with 100 g of indicated antibodies such as IgG1, KMTR2 and avelumab. Animals were injected with GSK269962A 2 mg/kg (in PBS) directly into tumors wherever ROCK1i is indicated. Data information: Mean±SD. Statistical significance in FIGS. 5E, FIG. 5G, FIG. 5I, FIG. 5L, FIG. 5M, and FIG. 5P was determined by unpaired two-tailed t-test and in FIG. 5N using two-tailed paired Wilcoxon Mann-Whitney test (n=4-6) (*p<0.05, **p<0.005, ***p<0.0001).

FIGS. 6A-6M. Co-targeting of DR5 with ROCK1i or PD-L1 enhances immune infiltration, overpowers immune suppression and improves anti-tumor activity. (FIG. 6A) Chi-G4S-DR5 stable 4T1 tumors harboring mice were treated lexatumumab, lexatumumab +ROCK1i, and avelumab+lexatumumab and other controls as indicated. Antibodies were treated i.p at 100 g dose (6 total), ROCK1i (in PBS) was injected directly. into tumors at 2 mg/kg dose (6 total). Various treatments were started when tumors were −400 mm³ size. Harvested tumors were grouped together and sized matched (3 independent sets: n=2-6 tumors in each set) followed by TIL isolation (see methods). CD8/CD45 and CD4/CD45 expressing cells were measured by flow cytometry. The data shown is from a single set of experiment. See also FIG. 24. (FIG. 6B) Plots showing % of total double positive TILs (CD8+CD45++CD4+CD45+) in right upper quadrant after combining 3 independent experiments. Indicated treatments are shown at the bottom of bars. (C) Ratio of CD8+CD45+/CD4+CD45+isolated TILs from tumors in each indicated treatment. (FIG. 6D) Similar to FIG. 6A Chi-G4S-DR5 stable 4T1 tumors harboring mice were treated (6 total doses) lexatumumab, avelumab, ROCK1i, lexatumumab+ROCKi, and avelumab+lexatumumab and IgG1 control as indicated. Harvested tumors homogenized followed by quantitation. Protein lysates were run on SDS-Page followed by immunoblotting using indicated CD8, CD4, Foxp3, caspase-3 and granzyme-b antibody. GAPDH is loading control. For additional Chi-G4S-DR5 stable MC38 tumors western data, see FIG. 18A. (FIGS. 6E-6G) Schematic and genetic construction of avelu-MD5-1 bispecific antibody that contains anti-PD-L1 (Avelumab), anti-muDR5 (MD5-1). Both monospecific and bispecific antibodies contain LALA mutation to avoid interference with Fc-effector function. (FIG. 6G) Working mechanism of avelu-MD5-1 bispecific antibody where surface stabilized PDL1 acts as an anchor to enhance avidity optimized binding and clustering of DR5 receptor mediated apoptotic signaling. (FIG. 6H) Cell killing assay of 4T1 cells treated with murine DR5 agonist MD5-1 and bispecific avelu-MD5 antibody. (FIG. 6I) Balb/c mice harboring luciferase stable 4T1 tumors were i.p. injected with indicated antibodies (100 and mice were imaged after 5 doses. (FIG. 6J) 6-8 weeks old C57BL/6 mice bearing MC38 tumors were intraperitoneally (i.p.) injected with 50 g of indicated antibody every third day (n=4-6). Indicated treatments were started when tumors were ˜100 mm³ size. Tumor volumes were quantified at indicated days by caliper measurements. (FIG. 6K) −400 mm³ size MC38 tumor bearing C57LB/6 mice were treated with indicated MD5-1, avelumab and bispecific antibodies along with control IgG1, 6 total doses. Harvested tumors were homogenized followed by quantitation. Protein lysates were run on SDSPage followed by immunoblotting using indicated CD8, CD4, caspase-3 and granzyme-b antibody. GAPDH is loading control. (FIG. 6L) 6-8 weeks old C57BL/6 mice were injected with MC38 tumors. When tumors reached −400 mm³, animals were intraperitoneally (i.p.) injected with 50 g of indicated antibody every third day. On day 18, tumors were harvested, sized matched and pooled by treatment group, exposed to collagenase/DNase and were single cell suspensions enriched for CD8+ cells. Enriched CD8+ T-cells from various treatments were restimulated with anti-CD3 (OKT3) antibody for 4 additional hours. CD8 gated cells were next analyzed for IFN-γ intracellular expression using flow cytometry. The data shown is from a single set of experiment. See also FIGS. 25A-25D. (FIG. 6M) Percentage of IFN-γ+CD8+ double positive cells from 3 independent experiments. For supporting flow cytometry data, see also FIGS. 25A-25D. Data information: Mean±SD. Statistical significance in FIG. 6B and FIG. 6C was determined by Mann Whitney two tailed test and in FIG. 6I using two-tailed paired Wilcoxon Mann-Whitney test. Statistical significance in FIG. 6M was determined by unpaired t test. (*p <0.05, **p<0.005, ***p<0.0001).

FIGS. 7A-7C. Working model of PD-L1 immunosuppression by DR5 agonist antibodies. Heterogeneous tumors consisting of DR5 sensitive (FIG. 7A), partially sensitive (FIG. 7B), and potentially resistant (FIG. 7C) tumor cells. DR5 agonist activates cell death above tumor clearance threshold in sensitive cells. Activation of caspase-8 and caspase-3 inactivates proteasome function and stabilizes intracellular PD-L1. Activated ROCK1 potentially help mobilizes PD-L1 to membrane by some unknown mechanism, which is also released in ApoEVs from dying cells. These PD-L1 containing ApoEVs shuttles and transfer cargo PD-L1 to other heterogenous cell types in tumors (potentially DR5 resistant) to increase the overall basal pool of PD-L1 in tumors. At the same time, due to extrinsic DR5 agonist mediated cytotoxicity, tumor cells are eliminated to generate partial tumor clearance and break down. However, incoming immune effector cells including T-cells are exhausted in the tumors due to overactive PD-L1, thus, limiting their anti-tumor response. Co-targeting ROCK1-DR5 reduces ApoEVs stabilized PD-L1 pool in tumors, while anti-PD-L1-DR5 cotargeting reduces immunosuppressive function of both basal and ApoEVs stabilized PDL1 in tumors.

FIGS. 8A-8G. DR5 agonist induced PD-L1 stabilization during transient epithelial to mesenchymal transitions. (FIG. 8A) Immunoblotting analysis of PD-L1, CD47 and calreticulin from MDA-MB-436 cell lysates treated with indicated DR5 agonist antibodies ±caspase inhibitor Z-VAD. GAPDH is loading control. (FIG. 8B) Immunoblotting of PD-L1, caspase-3 and PARP following surface biotinylation of PD-L1 from OVCAR-3 cells after indicated DR5 agonist treatments. (FIG. 8C) Flow cytometry analysis showing relative PD-L1 and CD47 surface intensity from indicated tumor cell lines treated with indicated DR5 agonists (normalized to IgG1 treatment). Since each cell lines have been treated with at least 3 DR5 agonist antibodies (lexatumumab, tigatuzumab, KMTR2, AMG655+Apo2L), normalized surface PD-L1 and CD47 represent biological replicate (n=3). (FIG. 8D) Immunoblotting of N-cadherin, E-cadherin, FOLR1 and vimentin from A549 cells after treatment with indicated growth factor (HGF, TNF-α and TGF-β) for indicated times to induce transient epithelial to mesenchymal transitions (EMTs). (FIG. 8E) Similar to FIG. 8C except OVCAR3 cells were used and were analyzed for surface DR5 expression prior and post transient EMT induction. (FIG. 8F) Cell viability assays of OVCAR3 and A549 cells in EMT and Non-EMT conditions after treatment with KMTR2. (FIG. 8G) PD-L1 flow cytometry analysis of OVCAR3 cells in non-EMT and EMT conditions after treatment with indicated DR5 agonist antibodies.

FIGS. 9A-9C. DR5 resistant tumors cells do not stabilize PD-L1 upon agonist antibody treatments. (FIG. 9A) Schematic of DR5 agonist resistant cell line generation. DR5 expressing WT cells were treated with varying concentrations of lexatumumab to select the resistant colonies. Selected colonies were continuously treated with lexatumumab to generate resistant clones. (FIG. 9B) Cell survival assays confirming generation of DR5 resistant cell lines. (FIG. 9C) Total PD-L1 and CD47 blotting analysis from DR5 resistant OVCAR-3 cell lines after indicated DR5 agonist treatment (50 nM, 6 hours).

FIGS. 10A-10D. A PD-L1 and PD-1 engaging tumor cell-jurkat cell co-culture reporter assay. (FIG. 10A) PD-1 effector Jurkat T cells stably express human PD-1 and NFAT-induced luciferase, while PD-L1 aAPC/CHO-K1 cells stably express human PD-L1 and a cell surface protein designed to activate cognate TCRs in an antigen-independent manner. Upon PD-1-PD-L1 interaction luciferase signal is downregulated. Antibodies blocking PD-1-PD-L1 interaction removes inhibitory signals, resulting in luciferase activation. (FIG. 10B) Modified PD-1-PD-L1 interaction model for our studies: (1) Engagement of CD3 activates luciferase in PD-1 effector jurkat reporter cells. (2) Upon DR5 agonist treatment, tumor cells mobilize PD-L1 on cell surface. (3) When DR5 agonist treated tumor cells are co-cultured with CD3 activated Jurkat cell, surface mobilized PD-L1 engages PD-1 on jurkat reporter cells leading to loss of luciferase activity. (FIG. 10C) Tumor cell-Jurkat cell co-culture assay in Colo-205, MDA-MB-231, U87 and A549 cells after treatment with indicated DR5 agonists. Because of their higher sensitivity to cell-death, for Colo-205 and U87 cells 100 nM conc. of DR5 agonist was used. Because of their lower level of sensitivity to cell-death, for MDA-MB-231 and A549 cells 500 nM conc. of DR5 agonist was used. (FIG. 10D) OVCAR3 cells treated with KMTR2 for indicated times were lysed and immunoblotted as indicated for PD-L1, PARP, CMTM6, Total p65, Total STAT3, Total ERK and E-cadherin.

FIGS. 11A-11G. PD-L1 stabilization by DR5 agonist does not require transcription and translation regulation and proteasome inhibition does not enhances surface PD-L1. (FIG. 11A) MDA-MB-436 cells were pretreated for 0, 2, 4 and 8 hours with actinomycin-D followed by KMTR2+lexa for 6 hours. Left 3 lanes are controls. Lysates were analyzed for PD-L1 levels. (FIG. 11B) MDA-MB-436 cells were pretreated for 0, 2, 4 and 8 hours with cycloheximide followed by KMTR2 for 6 hours. After 6 hours of KMTR2 treatment, lysates were analyzed for PD-L1 levels. (FIG. 11C) OVCAR-3 cells were treated with TNFα and indicated DR5 agonists for indicated times. Lysates were analyzed for PD-L1, CSN5, phosphorylated p65 and PARP. GAPDH is loading control. (FIG. 11D) PD-L1 flow cytometry analysis of MDA-MB-436 cells treated with Apo2L, lexa and KMTR2±MG132. (FIG. 11E)WT MDA-MB-231 cells or MDA-MB-231 DR5-KO cells treated with DR5 agonist (as indicated) and TNF-α next to each other followed by PD-L1 analysis using flow cytometry. (FIG. 11F) MDA-MB-436 cells were treated with indicated DR5 agonist±MG132. Lysates were analyzed for total PD-L1. GAPDH is loading control. (FIG. 11G) WT DR5 sensitive (DR5-S) and DR5 resistant (DR5−) MDA-MB-436 cells were treated for indicated times with KMTR2. Total lysates were analyzed for S5a/PSMD4, a subunit of 26S proteasome regulatory complex.

FIG. 12. TNF-α stabilized PD-L1 is not shuttled to ApoEVs. ApoEVs were isolated from DR5-WT MDA-MB-436 and OVCAR-3 cells after treatment with IgG1, TNF-α and lexatumumab. Isolated ApoEVs were incubated on to DR5-KO (MDA-MB-231) cells. After 24 hours surface PD-L1 of DR5-KO cells was analyze by flow cytometry. Top panel show secondary antibody control, IgG1 treatment control and basal level PD-L1 levels on MDA-MB-231 DR5 KO cells.

FIGS. 13A-13E. DR5 agonist activates ROCK1. (FIG. 13A) Tigatuzumab (anti-DR5) +anti-Fc treated MDA-MB-436 cell lysates for indicated early time points were analyzed for caspase-3, ROCK1, pMLC, PARP, PD-L1 and CMTM6 as indicated. Vertical arrows indicate sequential kinetics of caspase-3 and ROCK1 activation. (FIG. 13B) Lexatumumab treated Colo-205 cell lysates for indicated early time points were analyzed for cleaved caspase-8, ROCK1 and cleaved caspase-3. Vertical arrows indicate sequential kinetics of caspase-8, ROCK1 and caspase-3 activation. (FIG. 13C) Lexatumumab ±rDR5 treated MDA-MB-436 cell lysates for indicated time points were analyzed for cleaved caspase-8, ROCK1 and cleaved caspase-3. (FIGS. 13D-13F) Various clinical DR5 agonist antibody mediated cleavage and activation of ROCK1 in MDAMB-231 and U87 cells as confirmed by increased phosphorylation of myosin light chain, a ROCK1 substrate.

FIGS. 14A-14D. Schematic details of native immunoprecipitation and ROCK1 inhibition reduces PD-L1 activity in tumor cell-jurkat cell co-culture assays. (FIGS. 14A and 14B) Illustration of immunoprecipitation using clinical anti-PD-L1 avelumab antibody as described in FIGS. 4F-4H. Condition 1: IgG1 Fc containing DR5 agonists (KMTR2 or other controls) were added on tumor cells either alone or after pre-treatment of ROCK1 inhibitor (GSK269962A) for 6 hours. This was followed by lysates preparation in low salt RIPA buffer. Lysates were later incubated with IgG4 Fc containing avelumab for 1 hr, followed by pull down of avelumab using IgG4 Fc specific beads. Condition 2: IgG1 Fc containing DR5 agonists (KMTR2 or other controls) were added on tumor cells either alone or after pre-treatment of ROCK1 inhibitor (GSK269962A) for 6 hours. After 6 hours, IgG4 Fc containing avelumab was added to the media for additional 1 hr to bind to surface mobilized PD-L1. Lysates were made in low salt RIPA buffer, followed by pull down of avelumab using IgG4 Fc specific beads. (FIG. 14C) After separating the lysates on SDS-PAGE, immunoblotting was carried out using commercial PD-L1 antibody. In similar condition, another clinical anti-FOLR1, farletuzumab was used, followed by immunoblotting using commercial FOLR1 antibody. Only in condition 1, clinical antibodies pull down bound native protein complexes. (FIG. 14D) Tumor cell-Jurkat cell co-culture assay using A549, MDA-MB-231 and OVCAR3 tumors cells after treatment with indicated DR5 agonists alone or GSK269962 and GSK429286 (ROCK1 inhibitors) pretreated cells. Increased average luciferase intensity after ROCK1 inhibitor treatment from reporter cells confirmed decreased PD-L1 surface mobilization.

FIGS. 15A-15D. Confirmation of Chi-G4S-DR5 expression and both PD-1 and PD-L1 blockade improves anti-tumor function of DR5 agonists. (FIG. 15A) Chi-G4S-DR5 stable 4T1 cell were analyzed for DR5 expression using clinical tigatuzumab antibody in flow cytometry assay. (FIG. 15B) Chi-G4S-DR5 stable 4T1 tumors were treated with either single antibodies (IgG1, CD8 cells depleting anti-CD8a, avelumab, lexatumumab) or in combinations (avelumab +lexatumumab or avelumab+lexatumumab+anti-CD8a). 6, 100 μg dose of each antibodies was injected and tumors were harvested and imaged at same time. See FIG. 5M for tumor weight quanititation) (FIG. 15C) Chi-G4S-DR5 stable 4T1 tumors were treated with IgG1, lexatumumab and lexatumumab in combination of either avelumab (anti-PD-L1) or BMS202, a PD-1 inhibitor. After 6 doses, tumors were harvested and imaged at same time (FIG. 15D) Quantitation of average tumor weight as shown in FIG. 15C Data information: Mean±SD. Statistical significance in FIG. 15D was determined using two-tailed Mann-Whitney test. (*p<0.05, **p<0.005, ***p <0.0001).

FIG. 16. CD8+ and CD4+ T-cells tumor infiltration is enhanced upon DR5 agonist treatments. Chi-G4S-DR5 stable MC38 tumors harboring mice were treated (6 total doses) lexatumumab, avelumab, ROCK1i, lexatumumab+ROCKi, and avelumab+lexatumumab and IgG1 control as indicated. Mouse tumors were collected at 100-200 mm3 & embedded in O.C.T. to make blocks. Samples were processed and sectioned into 4 μm tissue sections. Tumor sections were stained with antibodies (CD8, CD4) and counter-stained with hematoxylin. Peroxidase conjugated anti-rabbit/anti-rat IgG reagents were used as secondary antibody. Reactions were developed using 3,3′-diaminobenzidine (DAB) as chromogenic substrate. Then, slides were dehydrated and mounted. Finally, brightfield images were taken using brightfield microscope. For quantification, 5-6 images were acquired at 20× magnification for each tumor sample.

FIG. 17. DR5 agonists do not enhance infiltration of regulatory T-cells in the tumors. Chi-G4S-DR5 stable MC38 tumors harboring mice were treated (6 total doses) lexatumumab, avelumab, ROCK1i, lexatumumab+ROCKi, and avelumab+lexatumumab and IgG1 control as indicated. Mouse tumors were collected at 100-200 mm3 & embedded in O.C.T. to make blocks and mouse spleen also collected & embedded as a positive control. Samples were processed and sectioned into 4 m tissue sections. Tumor sections were stained with antibodies (CD8, CD4) and counter-stained with hematoxylin. Peroxidase conjugated anti-rabbit/anti-rat IgG reagents were used as secondary antibody. Reactions were developed using 3,3′-diaminobenzidine (DAB) as chromogenic substrate. Then, slides were dehydrated and mounted. Finally, brightfield images were taken using brightfield microscope. For quantification, 5-6 images were acquired at 20× magnification for each tumor sample.

FIGS. 18A-18D. DR5+ROCK1 and DR5+PD-L1 targeting increases granzyme-b activity in tumors. (FIG. 18A) Chi-G4S-DR5 stable MC38 tumors harboring mice were treated (6 total doses) lexatumumab, avelumab, ROCK1i, lexatumumab+ROCKi, and avelumab+lexatumumab and IgG1 control as indicated. Harvested tumors homogenized followed by quantitation. Protein lysates were run on SDS-Page followed by immunoblotting using indicated CD8, CD4, Foxp3, and granzyme-b antibody. GAPDH is loading control. GAPDH is loading control. (FIG. 18B) FACs plots showing binding of anti-mouse DR5 antibody (MD5-1) and anti-mouse crossreactive clinical PD-L1 antibody (Avelumab) to 4T1 cells. (FIG. 18C) FACs plots showing binding of anti-mouse DR5 antibody (MD5-1) and anti-mouse crossreactive clinical PD-L1 antibody (Avelumab) to MC38 cells. (FIG. 18D) Cell killing assay of MC38 cells treated with murine DR5 agonist MD5-1 and bispecific avelu-MD5 antibody.

FIGS. 19A and 19B. Various solid tumor cells lines (MDA-MB-436, MDA-MB-231, OVCAR3, Cavo-3, U87, A549 etc.) treated with indicated DR5 agonist (±anti-Fc) for 4-8 hours were analyzed for surface PDL1 using flow cytometry. (B) MDA-MB-231, OVCAR3 treated with DR5 agonist for 4-6 hours were analyzed for surface CD47.

FIGS. 20A-20C. (FIG. 20A) 3 biological replicates of total PD-L1 and GAPDH from triple negative breast cancer cell (MDAMB-436) and ovarian cell (OVCAR-3) lysates treated with lexatumumab for 6 hours. (FIG. 20B) Raw mean fluorescent intensity (MFI) and % positive PD-L1 counts from the cells treated with indicated DR5 agonist antibodies. MFI values are shown on the top of solid color bars. There are multiple instances (shown on the top) where despite having lower % positive PD-L1 population, MFI values were higher after indicated DR5 agonist treatments. As an example, in MDA-MB-436 cells treated with tigatuzumab and lexatumumab, lexatumumab treated cells had higher % positive PD-L1 population (9.32 vs 11.97), however MFI was higher in tigatuzumab treated population (107.54 vs 45.87). (FIG. 20C) KMTR2 treated (50 μg, 4 doses) UCD52 TNBC PDX tumors were stained for PD-L1 using immunohistochemistry (IHC). 3 additional representative images are shown. Also see FIG. 1L.

FIGS. 21A-21C. (FIG. 2IA) 0.5×10⁶-2×10⁶ indicated tumor cells were injected subcutaneously in NOD.CgPrkdc^{scid}Il2rg^{tm1Wj1}/SzJ animals with matrigel in PBS. When tumors appeared on animals (3-4 weeks), animals were i.p. injected with indicated DR5 agonists (4-6 doses only), followed by tumor extraction and single cell suspension isolation from tumors after indicated antibody treatments. Isolated tumor cells were analyzed for surface PD-L1 using flow cytometry. (FIG. 2IB) Raw Data shown in FIG. 2IA was normalized with % positive PD-L1 in IgG1 treated tumors. (FIG. 2IC) Colo-205 tumor harboring athymic Nude Foxn1nu/Foxn1+(Envigo) xenografts with treated with IgG1, lexatumumab, KMTR2 and AMG655 (3 doses, 100 μg each). Harvested tumor lysates were analyzed for total PD-L1 expression. GAPDH is leading control.

FIG. 22. MDA-MB-436 and OVCAR3 cells were pre-treated with indicated ROCK1 inhibitors followed by addition of indicated DR5 agonists. Cells were later analyzed for surface PD-L1 using flow cytometry.

FIG. 23. Chi-G4S-DR5 stable 4T1 tumors were treated with indicated DR5 agonist either alone or in combination of avelumab (5 doses total). Recovered tumor cells were analyzed for surface PDL1 expression using flow cytometry.

FIG. 24. Chi-G4S-DR5 stable 4T1 tumors harboring mice were treated lexatumumab, lexatumumab+ROCK1i, and avelumab+lexatumumab and other controls as indicated. Antibodies were treated i.p at 100 g dose (6 total), ROCK1i (in PBS) was injected directly into tumors at 2 mg/kg dose (6 total). Harvested tumors were grouped together and sized matched (3 independent sets: n=2-6 tumors in each set) followed by TIL isolation (see methods). CD8/CD45 and CD4/CD45 expressing cells were measured by flow cytometry. The data shown is from two additional set of experiment. See also FIGS. 6A-6C.

FIGS. 25A-25D. (FIGS. 25A-25C) 6-8 weeks old C57BL/6 mice bearing MC38 tumors were intraperitoneally (i.p.) injected with 50 g of indicated antibody every third day. On day 18, tumors were harvested, sized matched and pooled by treatment group, exposed to collagenase/DNase and were single cell suspensions enriched for CD8+ cells. Enriched CD8+ T-cells from various treatments were restimulated with anti-CD3 (OKT3) antibody for 4 additional hours. CD8 gated cells were next analyzed for IFN-γ intracellular expression using flow cytometry. The data shown is from three additional set of experiments. See also FIGS. 6L and 6M. (FIG. 25D) ˜400 mm³ FVB/N-Tg(MMTV-PyVT)634Mul/J (Stock No: 002374, Jackson Laboratory) GEM tumors were extracted from animals and single cell suspension was isolated as described in FIG. 1I. Isolated tumor cells were analyzed for surface DR5 using flow cytometry (n=3).

### BRIEF DESCRIPTION OF THE SEQUENCE LISTING

SEQ ID NOs: 1 and 2 are the amino acid sequences of the lexatumumab lambda V_{L} and V_{H}regions, respectively.

SEQ ID NOs: 3 and 4 are the amino acid sequences of the tigatuzumab c-kappa V_{L} and V_{H}regions, respectively.

SEQ ID NOs: 5 and 6 are the amino acid sequences of the AMG-655 (Conatumumab) c-kappa V_{L} and V_{H} regions, respectively.

SEQ ID NOs: 7 and 8 are the amino acid sequences of the KMTR2 c-kappa V_{L} and V_{H}regions, respectively.

SEQ ID NOs: 9 and 10 are the amino acid sequences of the Avelumab c-kappa V_{L} and V_{H}regions, respectively.

SEQ ID NOs: 11 and 12 are the amino acid sequences of the Farletuzumab c-kappa V_{L} and VH regions, respectively.

SEQ ID NO: 13 is the amino acid sequence of a human DR5 polypeptide (HuDR5) that was employed in the experiments described herein.

SEQ ID NO: 14 is the amino acid sequence of a chimeric DR5 polypeptide (Chi-DR5) that was employed in the experiments described herein.

SEQ ID NO: 15 is the amino acid sequence of a chimeric DR5 polypeptide with a G4S (GGGGS; SEQ ID NO: 34) linker (Chi-G4S DR5) that was employed in the experiments described herein.

SEQ ID NOs: 16 and 17 are the nucleotide and amino acid sequences, respectively, of human ROCK1 gene products.

SEQ ID NOs: 18-21 are exemplary nucleotide and amino acid sequences of human CTLA4 gene products.

SEQ ID NOs: 22 and 23 are the nucleotide and amino acid sequences, respectively, of human PD-1 gene products.

SEQ ID NOs: 24-29 are exemplary nucleotide and amino acid sequences of human PD-L1 gene products.

SEQ ID NOs: 30-33 are exemplary nucleotide and amino acid sequences of human DR5 gene products.

SEQ ID NO: 34 is the amino acid sequence of a G4S linker.

### DETAILED DESCRIPTION

### I. General Considerations

Lack of effective immune infiltration represents a significant barrier to immunotherapy in solid tumors. Thus, solid tumor-enriched death receptor-5 (DR5) activating antibodies, which generates tumor debulking by extrinsic apoptotic cytotoxicity remains a crucial alternate therapeutic strategy. Over past few decades, many DR5 antibodies moved to clinical trials after successfully controlling tumors in immunodeficient tumor xenografts. However, DR5 antibodies failed to significantly improve survival in phase-II trials, leading in efforts to generate second generation of DR5 agonists to supersize apoptotic cytotoxicity in tumors. Disclosed herein is the discovery that clinical DR5 antibodies activate an unexpected immunosuppressive PD-L1 stabilization pathway, which potentially had contributed to their limited success in clinical trials. The DR5 agonist stimulated caspase-8 signaling not only activates ROCK1 but also undermines proteasome function, both of which contributes to increased PD-L1 stability on tumor cell surface. Targeting DR5-ROCK1-PD-L1 axis markedly increases immune effector T-cells function, promotes tumor regression, and improves overall survival in animal models. These insights have identified a potential clinically viable combinatorial strategy to revive solid cancer immunotherapy using death receptor agonism.

It is thus an object of the presently disclosed subject matter to provide methods and compositions for treating cancer. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and Figures.

### II. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in the following Table 1:

TABLE 1
Amino Acid Codes and Functionally Equivalent Codons

| | 3-Letter | 1-Letter | Functionally Equivalent |
|---|---|---|---|
| Full Name | Code | Code | Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

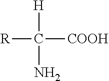

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ (scFv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in the following Table:

TABLE 2
Exemplary Conservative Amino Acid Substitutions

| Group | Characteristics | Amino Acids |
|---|---|---|
| A. | Small aliphatic, nonpolar or slightly | Ala, Ser, Thr, Pro, Gly |
| | polar residues | |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990a, modified as in Karlin & Altschul, 1993). This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane 1988 for a description of immunoassay formats and conditions that can be used to determine specific or selective immunoreactivity. See also the EXAMPLES set forth herein below for additional formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions.

Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2×standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### III. Compositions

In some embodiments, the presently disclosed subject matter relates to compositions that comprise at least two active agents. The first is an inhibitor of a biological activity of a Rho-Associated Coiled-Coil Containing Protein Kinase 1 (ROCK1) gene product and/or an immune checkpoint inhibitor, and the second is an agonist of a Death Receptor 5 (DR5; also known as TRAIL receptor 2 (TRAILR2) or tumor necrosis factor receptor superfamily member 10B (TNFRSF10B)) gene product.

The term “inhibitor” as used herein, refers to any compound or agent, the application of which results in the inhibition of a process or function of interest, including, but not limited to, a biological activity of a polypeptide and/or protein. Inhibition can be inferred if there is a reduction in the activity or function of interest.

In some embodiments, the compositions of the presently disclosed subject matter comprise an inhibitor of a biological activity of a ROCK1 gene product. As used herein, the phrase “Rho-Associated Coiled-Coil Containing Protein Kinase 1” (abbreviated “ROCK1”; also known as P160ROCK and ROKβ) refers to a genetic locus that encodes a serine/threonine kinase and its gene products. Exemplary human ROCK1 gene products include the nucleotide sequence disclosed as Accession No. NM_005406.3 (SEQ ID NO: 16) of the GENBANK® biosequence database, which encodes Accession No. NP_005397.1 (SEQ ID NO: 17) of the GENBANK® biosequence database. The human ROCK1 locus is found on chromosome 18 and corresponds to the reverse-complement of nucleotides 20,946,906-21,111,813 of Accession No. NC_000018.10 of the GENBANK® biosequence database. The ROCK1 protein is a protein kinase, and thus a “biological activity of a ROCK1 gene product” is an in vivo, in vitro, or ex vivo protein kinase activity that results from a ROCK1 polypeptide acting on a substrate to phosphorylate one or more amino acids present in the substrate. Exemplary inhibitors of ROCK1 include, but are not limited to small molecules that bind to ROCK1, antibodies and fragments thereof that bind to a ROCK1 polypeptide, and inhibitory nucleic acids (e.g., an siRNA, miRNA, or antisense RNA) that bind to ROCK1 nucleic acids, which as a result of the binding, inhibits to at least some degree a biological activity of the ROCK1 polypeptide. Non-limiting examples of small molecule ROCK1 inhibitors include GSK180736A (CAS No.: 817194-38-0), GSK-25 (CAS No.: 874119-56-9), Y-27632 dihydrochloride (CAS No.: 129830-38-2), GSK269962A (N-(3-{[2-(4-Amino-1,2,5-oxadiazol-3-yl)-1-ethyl-1H-imidazo[4,5-c]pyridin-6-yl]oxy}phenyl)-4-[2-(morpholin-4-yl)ethoxy]benzamide; CAS No.: 850664-21-0; also referred to as GSK269), and GSK429286A (N-(6-Fluoro-1H-indazol-5-yl)-6-methyl-2-oxo-4-[4-(trifluoromethyl)phenyl]-3,4-dihydro-1H-pyridine-5-carboxamide; CAS No.: 864082-47-3, also referred to as GSK429).

In some embodiments, an inhibitor of a biological activity of a ROCK1 gene product is a nucleic acid-based inhibitor, optionally an siRNA or an miRNA that targets a ROCK1 gene product (including but not limited to a nucleotide sequence disclosed as Accession No. NM_005406.3 (SEQ ID NO: 16) of the GENBANK® biosequence database).

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. siRNA technology has been described (see, for example, U.S. Pat. Nos. 6,506,559, 7,056,704, 8,420,391 and 8,372,968, which are incorporated herein by reference in their entirety).

The terms “microRNA” and “miRNA” are used interchangeably and refer to a nucleic acid molecule of about 17-24 nucleotides that is produced from a pri-miRNA, a pre-miRNA, or a functional equivalent. miRNAs are to be contrasted with short interfering RNAs (siRNAs), although in the context of exogenously supplied miRNAs and siRNAs, this distinction might be somewhat artificial. The distinction to keep in mind is that a miRNA is necessarily the product of nuclease activity on a hairpin molecule such as has been described herein, and an siRNA can be generated from a fully double-stranded RNA molecule or a hairpin molecule. Further information related to miRNAs generally, as well as a database of known published miRNAs and searching tools for mining the database can be found at the Wellcome Trust Sanger Institute miRBase: Sequences website, herein incorporated by reference. See also Griffiths-Jones, 2004, herein incorporated by reference. miRNA technology has been described (see, for example, U.S. Pat. Nos. 7,960,359, 7,825,230, 7,825,229 and 7,592,441, which are incorporated herein by reference in their entirety).

As used herein, the term “RNA” refers to a molecule comprising at least one ribonucleotide residue. By “ribonucleotide” is meant a nucleotide with a hydroxyl group at the 2′ position of a 0-D-ribofuranose moiety. The terms encompass double stranded RNA, single stranded RNA, RNAs with both double stranded and single stranded regions, isolated RNA such as partially purified RNA, essentially pure RNA, synthetic RNA, and recombinantly produced RNA. Thus, RNAs include, but are not limited to mRNA transcripts, miRNAs and miRNA precursors, and siRNAs. As used herein, the term “RNA” is also intended to encompass altered RNA, or analog RNA, which are RNAs that differ from naturally occurring RNA by the addition, deletion, substitution, and/or alteration of one or more nucleotides. Such alterations can include addition of non-nucleotide material, such as to the end(s) of the RNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs. In some embodiments, the double stranded region is at least 15 basepairs, in some embodiments between 15 and 300 basepairs, and in some embodiments between 15 and about 60 basepairs. As describe hereinabove, the formation of the double stranded region results from the hybridization of complementary RNA strands (for example, a sense strand and an antisense strand), either via an intermolecular hybridization (i.e., involving 2 or more distinct RNA molecules) or via an intramolecular hybridization, the latter of which can occur when a single RNA molecule contains self-complementary regions that are capable of hybridizing to each other on the same RNA molecule. These self-complementary regions are typically separated by a short stretch of nucleotides (for example, about 5-10 nucleotides) such that the intramolecular hybridization event forms what is referred to in the art as a “hairpin” or a “stem-loop structure.”

In some embodiments, the compositions of the presently disclosed subject matter comprise an immune checkpoint inhibitor. As used herein the phrase “immune checkpoint inhibitor” refers to any molecule such as but not limited to a small molecule, an antibody or fragment thereof that includes a paratope that binds to an immune checkpoint polypeptide, or an inhibitory RNA that binds to an immune checkpoint polypeptide to interfere with an interaction between an inhibitory receptor and its ligand, wherein the inhibitory receptor is essential to balance co-stimulatory receptor activity and limit T-cell activation. Thus, such an antibody, small molecule, or inhibitory nucleic acid targets immune system checkpoints such as the cytotoxic T-lymphocyte antigen 4 (CTLA-4), the programmed death-1 (PD-1) or its ligand (PD-L1). Examples of immune checkpoint inhibitory antibodies suitable for use in the compositions of the presently disclosed subject matter, without being limited thereto, are anti-PD-1 antibodies, anti-PD-L1 antibodies, anti-IDO antibodies, anti-CTLA-4 antibodies, anti-Tim-3 antibodies, anti-GITR antibodies, anti-OX40 antibodies, or anti-LAG3 antibodies. In most cases, the immune checkpoint inhibitory antibody is an antagonistic or blocking antibody, i.e., a blocking antibody selected from the group consisting of an anti-PD-1 antibody, an anti-PD-L1 antibody, an anti-IDO antibody, an anti-CTLA-4 antibody, an anti-Tim-3 antibody, an anti-LAG3 antibody, an anti-VISTA antibody, and an anti-BTLA antibody. However, an immune checkpoint inhibitory antibody may also be an agonistic antibody, such as an anti-GITR or an anti-OX40 antibody. With regard to the anti-LAG3 antibody, this antibody can be an antibody selectively inhibiting the interaction of LAG3 and MHC class II molecule or an antibody selectively inhibiting the interaction of LAG3 and FGL1 or an antibody inhibiting the interaction of LAG3 with FGL1 and MHC class II molecule. In some embodiments, the immune checkpoint inhibitory antibody is an IgG antibody, in some embodiments a human or a humanized IgG antibody, and in some embodiments a human or a humanized IgG1 or IgG4 antibody.

As used herein, the phrase “Cytotoxic T-Lymphocyte Associated Protein 4” (abbreviated “CTLA4”; also known as CD152) refers to a member of the immunoglobulin superfamily that is expressed by activated T cells and transmits inhibitory signals to T cells. Exemplary human CTLA4 gene products include the nucleotide sequences disclosed as Accession Nos. NM_005214.5 (SEQ ID NO: 18) and NM_001037631.3 (SEQ ID NO: 20) of the GENBANK® biosequence database, which encode Accession Nos. NP_005205.2 (SEQ ID NO: 19) and NP_001032720.1 (SEQ ID NO: 21) of the GENBANK® biosequence database, respectively. The human CTLA4 locus is found on chromosome 2 and corresponds to nucleotides 203,867,771-203,873,965 of Accession No. NC_000002.12 of the GENBANK® biosequence database.

As used herein, the phrase “Programmed Cell Death Protein 1” (abbreviated “PD-1”; also known as CD279) refers to an immune-inhibitory receptor-encoding gene that is expressed in activated T cells. PD-1 is involved in T cell regulation, particularly with respect to effector CD8+ T cells. Exemplary human PD-1 gene products include the nucleotide sequence disclosed as Accession No. NM_005018.3 (SEQ ID NO: 22) of the GENBANK® biosequence database, which encodes Accession No. NP_005009.2 (SEQ ID NO: 23) of the GENBANK® biosequence database. See Okazaki et al., 2002; Bennett et al., 2003.

As used herein, the phrase “Programmed Death-Ligand 1” (abbreviated “PD-L1”; also known as CD274) refers to a gene that encodes a ligand that binds to PD-1 to block T cell activation. Exemplary human PD-L1 gene products include the nucleotide sequences disclosed as Accession Nos. NM_001314029.2 (SEQ ID NO: 24), NM_001267706.2 (SEQ ID NO: 26), and NM_014143.4 (SEQ ID NO: 28) of the GENBANK® biosequence database, which encode Accession Nos. NP_001300958.1 (SEQ ID NO: 25), NP_001254635.1 (SEQ ID NO: 27), and NP_054862.1 (SEQ ID NO: 29) of the GENBANK® biosequence database, respectively. See Agata et al., 1996.

Similarly to the discussion herein above with respect to ROCK1 inhibitors, an immune checkpoint inhibitor (e.g., a CTLA4 inhibitor, a PD-1 inhibitor, a PD-L1 inhibitor) can also be a small molecule that binds to and at least partially inhibits a biological activity of an immune checkpoint protein or a nucleic acid encoding the same. Exemplary small molecule inhibitors include those disclosed in Zhang et al., 2020; U.S. Patent Application Publication No. 2020/0155521 (e.g., PD-1/PD-L1 Inhibitor 3, BMS202, AUNP-12, and PD-1/PD-L1 inhibitor 1); and U.S. Pat. No. 10,684,287; each of which is incorporated herein by reference in its entirety. Exemplary inhibitory nucleic acids include those that target an immune checkpoint gene product (including but not limited to a nucleotide sequence disclosed as Accession No. NM_005406.3 of the GENBANK® biosequence database)

An immune checkpoint inhibitor can also be an antibody or a fragment thereof that binds to and at least partially inhibits a biological activity of an immune checkpoint protein. Exemplary antibody-based immune checkpoint inhibitors include but are not limited to those described in U.S. Pat. No. 10,441,655 (incorporated herein by reference in its entirety); pembrolizumab, nivolumab, cemiplimab, atezolizumab, dostarlimab, durvalumab, avelumab, balstilimab, Bavencio, Keytruda, Libtayo, Opdivo, penpulimab, retifanlimab, sintilimab, Tecentriq, and Tyvyt.

In some embodiments, an immune checkpoint inhibitor can comprise a nucleic acid-based inhibitor, optionally an siRNA or an miRNA, that targets an immune checkpoint inhibitor gene product (e.g., a CTLA4, PD-1, or PD-L1 mRNA). Exemplary immune checkpoint inhibitor gene products include but are not limited to nucleotide sequences disclosed as Accession Nos. NM_005214.5 (SEQ ID NO: 18) and NM_001037631.3 (CTLA4), NM_005018.3 (SEQ ID NO: 22; PD-1), and NM_001314029.2 (SEQ ID NO: 24), NM_001267706.2 (SEQ ID NO: 26), and NM_014143.4 ((SEQ ID NO: 24; PD-L1) of the GENBANK® biosequence database.

In some embodiments, the compositions of the presently disclosed subject matter also comprise a Death Receptor 5 (DR5) agonist. As used herein, the term “Death Receptor 5” (abbreviated as “DR5”; also known as TRAIL receptor 2 (TRAILR2) and tumor necrosis factor receptor superfamily member 10B (TNFRSF10B)) refers to a gene encoding a cell surface receptor of the TNF-receptor superfamily that binds TRAIL and mediates apoptosis and its products. Exemplary human DR5 gene products include the nucleotide sequences disclosed as Accession Nos. NM_003842.5 (SEQ ID NO: 30) and NM_147187.3 (SEQ ID NO: 32) of the GENBANK® biosequence database, which encode Accession Nos. NP_003833.4, (SEQ ID NO: 31) and NP_671716.2 (SEQ ID NO: 33) of the GENBANK® biosequence database, respectively. Exemplary DR5 agonists include lexatumumab, apomab, AMG655 (also called conatumumab), KMTR2 (see Tamada et al., 2015), and tigatuzumab (see Forero-Torres et al., 2013).

In some embodiments, the presently disclosed compositions comprise, consist essentially of, or consist of a bispecific antibody. As used herein, the phrase “bispecific antibody” refers to an antibody having binding specificities for at least two different antigenic epitopes. In some embodiments, the epitopes are from the same antigen. In some embodiments, the epitopes are from two different antigens. In some embodiments, the epitopes are from a ROCK1 polypeptide and/or an immune checkpoint polypeptide on the one hand and a DR5 polypeptide on the other. Methods for making bispecific antibodies are known in the art. For example, bispecific antibodies can be produced using recombinant technology using the co-expression of two immunoglobulin heavy chain/light chain pairs. See e.g., Milstein & Cuello, 1983. Alternatively, bispecific antibodies can be prepared using chemical linkage. See e.g., Brennan et al., 1985. Bispecific antibodies include bispecific antibody fragments. See e.g., Gruber et al., 1994. In some embodiments, a composition of the presently disclosed subject matter further comprises, consists essentially of, or consists of a pharmaceutically acceptable carrier, optionally a pharmaceutically acceptable carrier that is pharmaceutically acceptable for use in a human.

As such, in some embodiments the presently disclosed subject matter relates to bispecific antibodies and fragments thereof. In some embodiments, the bispecific antibodies and fragments thereof include a first binding function that binds to a death receptor 5 (DR5) polypeptide and second binding function that binds to a polypeptide selected from the group consisting of a Rho-Associated Coiled-Coil Containing Protein Kinase 1 (ROCK1) polypeptide, a Cytotoxic T-Lymphocyte Associated Protein 4 (CTLA4) polypeptide, a Programmed Cell Death Protein 1 (PD-1) polypeptide, and a Programmed Death-Ligand 1 (PD-L1) polypeptide, wherein said bispecific antibody comprises a first antigen binding site that is specific for the DR5 polypeptide and a second antigen binding site that is specific for the ROCK1 polypeptide, the CTLA4 polypeptide, the PD-1 polypeptide, or the PD-L1 polypeptide. In some embodiments, the bispecific antibody and/or the fragment thereof comprises a heavy chain variable region and/or a light chain variable as set forth in any of SEQ ID NOs: 1-12. In some embodiments, a bispecific antibody of the presently disclosed subject matter is humanized. In some embodiments, a bispecific antibody of the presently disclosed subject matter further comprises a pharmaceutically acceptable carrier, optionally a pharmaceutically acceptable carrier that is pharmaceutically acceptable for use in a human.

Accordingly, in some embodiments the presently disclosed subject matter relates to compositions for use in treating tumors and/or cancers. In some embodiments, the compositions comprise, consist essentially of, or consist of (a) an effective amount of an inhibitor of a Rho-Associated Coiled-Coil Containing Protein Kinase 1 (ROCK1) biological activity and/or an effective amount of a checkpoint inhibitor (optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor), or any combination thereof, and (b) an effective amount of a Death Receptor 5 (DR5) agonist. In some embodiments, the composition comprises, consists essentially of, or consists of a bispecific antibody, and further wherein the bispecific antibody comprises a first antigen binding site that is specific for the DR5 polypeptide and a second antigen binding site that is specific for a ROCK1 polypeptide, a CTLA4 polypeptide, a PD-1 polypeptide, and/or a PD-L1 polypeptide. In some embodiments, the composition for use of the presently disclosed subject matter further comprises, consists essentially of, or consists of a pharmaceutically acceptable carrier, optionally a pharmaceutically acceptable carrier that is pharmaceutically acceptable for use in a human.

Similarly, in some embodiments the presently disclosed subject matter relates to bispecific antibodies for use in treating tumors and/or cancers, which in some embodiments comprise, consist essentially of, or consist of a first binding activity that binds to a death receptor 5 (DR5) polypeptide and a second binding activity that binds to a ROCK1 polypeptide, a CTLA4 polypeptide, a PD-1 polypeptide, and/or a PD-L1 polypeptide. In some embodiments, a bispecific antibody of the presently disclosed subject matter comprises a heavy chain variable region and/or a light chain variable as set forth in any of SEQ ID NOs: 1-12. In some embodiments, the bispecific antibody is humanized. In some embodiments, a bispecific antibody for use in treating a tumor and/or a cancer of the presently disclosed subject matter further comprises, consists essentially of, or consists of a pharmaceutically acceptable carrier, optionally a pharmaceutically acceptable carrier that is pharmaceutically acceptable for use in a human.

One of ordinary skill in the art will appreciate that based on the sequences of the components of the antibodies disclosed herein they can be modified independently of one another with conservative amino acid changes, including, insertions, deletions, and substitutions, and that the valency could be altered as well. Amino acid changes (fragments and homologs) can be made independently in an antibody as well when they are being used in a therapy.

The presently disclosed subject matter provides other antibodies and biologically active fragments and homologs thereof as well as methods for preparing and testing new antibodies for the properties disclosed herein.

In some embodiments, the fragments are fragments of scFv. In some embodiments, the scFv fragments are mammalian. In some embodiments, the scFv fragments are humanized.

In some embodiments, the presently disclosed subject matter uses a biologically active antibody or biologically active fragment or homolog thereof. In some embodiments, the isolated polypeptide comprises a mammalian molecule at least about 30% homologous to a polypeptide having the amino acid sequence of at least one of the sequences disclosed herein. In some embodiments, the isolated polypeptide is at least about 35% homologous, more in some embodiments, about 40% homologous, more in some embodiments, about 45% homologous, in some embodiments, about 50% homologous, more in some embodiments, about 55% homologous, in some embodiments, about 60% homologous, more in some embodiments, about 65% homologous, in some embodiments, more in some embodiments, about 70% homologous, more in some embodiments, about 75% homologous, in some embodiments, about 80% homologous, more in some embodiments, about 85% homologous, more in some embodiments, about 90% homologous, in some embodiments, about 95% homologous, more in some embodiments, about 96% homologous, more in some embodiments, about 97% homologous, more in some embodiments, about 98% homologous, and most in some embodiments, about 99% homologous to at least one of the peptide sequences disclosed herein.

The presently disclosed subject matter further encompasses modification of the antibodies and fragments thereof disclosed herein, including amino acid deletions, additions, and substitutions, particularly conservative substitutions. The presently disclosed subject matter also encompasses modifications to increase in vivo half-life and decrease degradation in vivo. Substitutions, additions, and deletions can include, for example, 1, 2, 3, 4, 5, 6, 7, 8, 9, 10, 11, 12, 13, 14, 15, 16, 17, 18, 19, 20, 21, 22, 23, 24, and 25 changes as long as the activity disclosed herein remains substantially the same.

The presently disclosed subject matter includes an isolated nucleic acid comprising a nucleic acid sequence encoding an antibody of the presently disclosed subject matter, or a fragment or homolog thereof. In some embodiments, the nucleic acid sequence encodes a peptide comprising an antibody sequence of the presently disclosed subject matter, or a biologically active fragment of homolog thereof.

In some embodiments, a homolog of a peptide (antibody or fragment) of the presently disclosed subject matter is one with one or more amino acid substitutions, deletions, or additions, and with the sequence identities described herein. In some embodiments, the substitution, deletion, or addition is conservative.

In some embodiments, the subject is a mammal. In some embodiments, the mammal is a human.

The presently disclosed subject matter encompasses the use of purified isolated, recombinant, and synthetic peptides.

Peptide Modification and Preparation

It will be appreciated, of course, that the proteins or peptides of the presently disclosed subject matter may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

The presently disclosed subject matter also provides for analogs of proteins, e.g., analogs of antibodies. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the presently disclosed subject matter are not limited to products of any of the specific exemplary processes listed herein.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus.

Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al., 1990.

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

Amino Acid Substitutions. In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C₁-C₁₀ carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, amino acids whose hydropathic indices are within in some embodiments ±2, in some embodiments within ±1, and in some embodiments within ±0.5 can be employed.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr; Pro (P) Ala; Ser (S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. See e.g., the PROWL website of Rockefeller University, New York, New York, United States of America. For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix.

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Antibody Formats and Preparation Thereof

Antibodies directed against proteins, polypeptides, or peptide fragments thereof of the presently disclosed subject matter may be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab.

For the preparation of monoclonal antibodies, any technique which provides for the production of antibody molecules by continuous cell lines in culture may be utilized. For example, the hybridoma technique originally developed in 1975 by Kohler and Milstein (Kohler & Milstein, 1975), the trioma technique, the human B-cell hybridoma technique (Kozbor & Roder, 1983), and the EBV-hybridoma technique (Cole et al., 1985) may be employed to produce human monoclonal antibodies. In some embodiments, monoclonal antibodies are produced in germ-free animals.

In accordance with the presently disclosed subject matter, human antibodies may be used and obtained by utilizing human hybridomas (Cote et al., 1983) or by transforming human B cells with EBV virus in vitro (Cole et al., 1985). Furthermore, techniques developed for the production of “chimeric antibodies” (Morrison et al., 1984; Neuberger et al., 1984; Takeda et al., 1985) by splicing the genes from a mouse antibody molecule specific for epitopes of SLLP polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

Various techniques have been developed for the production of antibody fragments of humanized antibodies. Traditionally, these fragments were derived via proteolytic digestion of full-length antibodies (see e.g., Morimoto & Inouye, 1992; Brennan et al., 1985). However, these fragments can now be produced directly by recombinant host cells. Alternatively, Fab′-SH fragments can be directly recovered from E. coli and chemically coupled to form F(ab′)₂ fragments (Carter et al., 1992a). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). See PCT International Patent Application Publication No. WO 1993/16185; U.S. Pat. Nos. 5,571,894; 5,587,458. The antibody fragment may also be a “linear antibody”, e.g., as described in U.S. Pat. No. 5,641,870, for example. Such linear antibody fragments may be monospecific or bispecific.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see e.g., U.S. Pat. Nos. 4,975,369; 5,075,431; 5,081,235; 5,169,939; 5,202,238; 5,204,244; 5,231,026; 5,292,867; 5,354,847; 5,472,693; 5,482,856; 5,491,088; 5,500,362; and 5,502,167). Detailed methods for preparation of chimeric (humanized) antibodies can be found in U.S. Pat. No. 5,482,856. A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see e.g., Jones et al., 1986; Riechmann et al., 1988; Presta, 1992, PCT International Patent Application Publication No. WO 92/02190, U.S. Patent Application Publication No. 2006/0073137, and U.S. Pat. Nos. 5,225,539; 5,530,101; 5,585,089; 5,693,761; 5,693,762; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; and 6,750,325.

In some embodiments, the presently disclosed subject matter provides for fully human antibodies. Human antibodies consist entirely of characteristically human polypeptide sequences. The human antibodies of this presently disclosed subject matter can be produced in using a wide variety of methods (see e.g., U.S. Pat. No. 5,001,065, for review).

Typically, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., 1986; Riechmann et al., 1988; Verhoeyen et al., 1988), by substituting hypervariable region sequences for the corresponding sequences of a human “acceptor” antibody. Accordingly, such “humanized” antibodies are chimeric antibodies (see e.g., U.S. Pat. Nos. 4,816,567 and 5,482,856) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

Another method for making humanized antibodies is described in U.S. Patent Application Publication No. 2003/0017534, wherein humanized antibodies and antibody preparations are produced from transgenic non-human animals. The non-human animals are genetically engineered to contain one or more humanized immunoglobulin loci that are capable of undergoing gene rearrangement and gene conversion in the transgenic non-human animals to produce diversified humanized immunoglobulins.

In some embodiments, the choice of human variable domains, both light and heavy, to be used in making the humanized antibodies is very important to reduce antigenicity. According to the so-called “best-fit” method, the sequence of the variable domain of a rodent antibody is screened against a library of known human variable-domain sequences or a library of human germline sequences. The human sequence that is closest to that of the rodent can then be accepted as the human framework region for the humanized antibody (Sims et al., 1993; Chothia & Lesk, 1987). Another method uses a particular framework region derived from the consensus sequence of all human antibodies of a particular subgroup of light or heavy chains. The same framework may be used for several different humanized antibodies (Carter et al., 1992b; Presta et al., 1993). Other methods designed to reduce the immunogenicity of the antibody molecule in a human patient include veneered antibodies (see e.g., U.S. Pat. No. 6,797,492 and U.S. Patent Application Publication Nos. 2002/0034765 and 2004/0253645) and antibodies that have been modified by T-cell epitope analysis and removal (see e.g., U.S. Patent Application Publication No. 2003/0153043 and U.S. Pat. No. 5,712,120).

It is important that when antibodies are humanized they retain high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The antibody moieties of this presently disclosed subject matter can be single chain antibodies.

The hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See for example, U.S. Patent Application Publication No. 2003/0022244.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments.

In some embodiments, the specific binding molecule is a single-chain variable analogue (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies, tri-specific such as triabodies and tetra-specific such as tetrabodies when each scFv in the dimer, trimer, or tetramer has a different specificity. Diabodies, triabodies and tetrabodies can also be monospecific, when each scFv in the dimer, trimer, or tetramer has the same specificity.

In some embodiments, techniques described for the production of single-chain antibodies (U.S. Pat. No. 4,946,778, incorporated by reference herein in its entirety) are adapted to produce protein-specific single-chain antibodies. In some embodiments, the techniques described for the construction of Fab expression libraries (Huse et al., 1989) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which bind the antigen therefrom at any epitopes present therein.

Monoclonal antibodies directed against full length or peptide fragments of a protein or peptide may be prepared using any well known monoclonal antibody preparation procedures, such as those described, for example, in Harlow & Lane, 1988; Tuszynski et al., 1988). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Exemplary complementarity-determining region (CDR) residues or sequences and/or sites for amino acid substitutions in framework region (FR) of such humanized antibodies having improved properties such as, e.g., lower immunogenicity, improved antigen-binding or other functional properties, and/or improved physicochemical properties such as, e.g., better stability, are provided.

The presently disclosed subject matter encompasses more than the specific fragments and humanized fragments disclosed herein. In some embodiments, the antibody is selected from the group consisting of a single chain antibody, a monoclonal antibody, a bi-specific antibody, a chimeric antibody, a synthetic antibody, a polyclonal antibody, or a humanized antibody, or active fragments or homologs thereof.

A nucleic acid encoding the monoclonal antibody obtained using the procedures described herein may be cloned and sequenced using technology which is available in the art, and is described, for example, in Wright et al., 1992) and the references cited therein. Further, the antibody of the presently disclosed subject matter may be “humanized” using the technology described in Wright et al., 1992 and in the references cited therein, and in Gu et al., 1997.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Green & Sambrook, 2012.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton & Barbas, 1994). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions may be synthesized such that they include nearly all possible specificities (Barbas, 1995; de Kruif et al., 1995).

In the production of antibodies, screening for the desired antibody can be accomplished by techniques known in the art, e.g., ELISA (enzyme-linked immunosorbent assay). Antibodies generated in accordance with the presently disclosed subject matter may include, but are not limited to, polyclonal, monoclonal, chimeric (i.e., “humanized”), and single chain (recombinant) antibodies, Fab fragments, and fragments produced by a Fab expression library.

It is common in the field of recombinant humanized antibodies to graft murine CDR sequences onto a well-established human immunoglobulin framework previously used in human therapies such as the framework regions of Herceptin [Trastuzumab].

In some embodiments, when used in vivo for therapy, the antibodies of the subject presently disclosed subject matter are administered to the subject in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks. Optionally, the administration is made during the course of adjunct therapy such as antimicrobial treatment, or administration of tumor necrosis factor, interferon, or other cytoprotective or immunomodulatory agent.

In some embodiments, for parenteral administration, the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

Pharmaceutical Compositions and Administration

The presently disclosed subject matter is also directed to methods of administering the compounds of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method for treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the peptides of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The peptides of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection (e.g., peri-tumoral and intra-tumoral injection), subcutaneous injection or deposition including subcutaneous infusion (such as by osmotic pumps), intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, 1985, which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the compound be used cooperatively by the recipient.

### IV. Methods and Uses

The presently disclosed subject matter also provides in some embodiments methods for treating tumors and/or cancers in subjects in need thereof. In some embodiments, the methods comprise, consist essentially of, or consists of administering to a subject in need thereof a composition comprising, consisting essentially of, or consists of an effective amount of an inhibitor of a Rho-Associated Coiled-Coil Containing Protein Kinase 1 (ROCK1) biological activity and/or an effective amount of a checkpoint inhibitor, optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor, or any combination thereof, and (b) a composition comprising an effective amount of a Death Receptor 5 (DR5) agonist. In some embodiments, the tumor and/or the cancer comprises a solid tumor, optionally a solid tumor selected from the group consisting of an ovarian tumor, a glioblastoma, a pancreatic tumor, a lung tumor, and a triple negative breast (TNBC) tumor.

In some embodiments of the presently disclosed methods, the inhibitor of ROCK1 activity is a small molecule inhibitor. In some embodiments, the inhibitor of ROCK1 activity is selected from the group comprising N-(3-{[2-(4-Amino-1,2,5-oxadiazol-3-yl)-1-ethyl-1H-imidazo[4,5-c]pyridin-6-yl]oxy}phenyl)-4-[2-(morpholin-4-yl)ethoxy]benzamide (i.e., GSK269) and N-(6-Fluoro-1H-indazol-5-yl)-6-methyl-2-oxo-4-[4-(trifluoromethyl)phenyl]-3,4-dihydro-1H-pyridine-5-carboxamide (i.e., GSK429).

In some embodiments of the presently disclosed methods, the checkpoint inhibitor comprises an antibody, optionally an antibody that binds to a CTLA4 polypeptide, a PD-1 polypeptide, and/or a PD-L1 polypeptide. Exemplary antibodies that can be employed in the methods of the presently disclosed subject matter include avelumab, atezolizumab, durvalumab, nivolumab, pembrolizumab, spartalizumab, tremelimumab, and ipilimumab, or any combination thereof.

In some embodiments of the presently disclosed methods, the DR5 agonist comprises a DR5 targeting antibody. Exemplary DR5 targeting antibodies include lexatumumab, apomab, AMG655, LBy135 (Li et al., 2007), WD-1 (Wang et al., 2008), KMTR2, and tigatuzumab.

Accordingly, in some embodiments of the presently disclosed methods, the composition comprises (a) an effective amount of an inhibitor of a ROCK1 biological activity and/or an effective amount of a checkpoint inhibitor, optionally wherein the checkpoint inhibitor is a CTLA4 inhibitor, a PD-1 inhibitor, a PD-L1 inhibitor, or any combination thereof, and (b) an effective amount of a DR5 agonist in a single composition.

In some embodiments, the presently disclosed subject matter provides for the use of (a) a composition comprising an effective amount of an inhibitor of a Rho-Associated Coiled-Coil Containing Protein Kinase 1 (ROCK1) biological activity and/or an effective amount of a checkpoint inhibitor, optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor, or any combination thereof, and (b) a composition comprising an effective amount of a Death Receptor 5 (DR5) agonist in the preparation of a medicament for treating a tumor and/or a cancer in a subject in need thereof. The medicament can be configured to administer components (a) and (b) in a single composition or in separate compositions.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Introduction to the Examples

T-cell activating monoclonal and bispecific antibodies have shown great potential to enhance immunotherapy against tumors and have led to collective decline in overall death rates from cancer (Mellman et al., 2011; Brinkmann & Kontermann, 2017). Unfortunately, in case of solid tumors, the average clinical response of cancer immunotherapy and chimeric antigen receptor T-cells (CAR-T) based strategies is significantly lower as compared to leukemia and melanoma (Melero et al., 2014). Other than factors contributing to cellular and genetic tumor heterogeneity, immune exhaustion and limited infiltration of immune effector cells in solid tumor microenvironment (called immune “cold” tumors) remains the bottleneck for observed lower immunotherapy responses (Anderson et al., 2017; Melero et al., 2014). In support various immune checkpoint targeting therapies work effectively against “immune hot tumors” that are adequately infiltrated with effector T-cells (Haanen, 2017). Furthermore, surgical tumor debulking (Guisier et al., 2019) and combinatorial approaches such as cytotoxic drugs (and neoadjuvants) also work via effective breakdown of solid tumor mass (Opzoomer et al., 2019), which orchestrates immunogenic tumor microenvironment (Obeid et al., 2007) and enhances increased T-cell infiltration to improve antitumor response.

Unlike neoadjuvant and chemotherapy drugs, extrinsic apoptotic pathways instigate tumor breakdown and clearance without the associated toxicity (Narayan & Vaughn, 2015). Therefore, pro-apoptotic receptor agonists (PARA) therapy using Trail ligand (Apo2L) or epithelial cancer enriched death receptor 5 (DR5/TRAIL-R2) activating antibodies gained significant attention in early twentieth century. PARA activate extrinsic apoptotic pathway by oligomerizing DR5, a hallmark of TNF receptor superfamily members (Ashkenazi, 2015). Importantly, PARA preferably activate extrinsic cell death in p53 mutant cancer cells (Ashkenazi & Herbst, 2008; Wu et al., 1997). As >75% of solid tumors carry p53 loss of function mutations, multiple DR5 agonist antibodies: lexatumumab (Marini, 2006), apomab (Camidge, 2008), AMG655 (Graves et al., 2014), Tigatuzumab (Forero-Torres et al., 2015) have been tested clinically after proven effective in various immunodeficient xenograft solid tumor models (Camidge, 2008; Kaplan-Lefko et al., 2010; Tamada et al., 2015). Recent efforts are also directed to generate and test the second-generation of DR5 activating approaches (Tamada et al., 2015; Wajant, 2019). Other reports have also described Apo2L ligand-agonist antibody co-targeting and bispecific antibody-based approaches to increase anti-tumor DR5 signaling (Graves et al., 2014; Shivange et al., 2018; Wajant, 2019).

Sadly, all clinically tested DR5 agonist antibodies so far have failed to improve survival in phase-II trials even when given in combination of nanoparticle albumin-bound paclitaxel (nab-paclitaxel) neoadjuvant therapy against high DR5 expressing TNBC patients (Forero-Torres et al., 2015). On the contrary, nab-paclitaxel therapy has significantly improved the survival of metastatic TNBC patients if given in combination of antiprogrammed death-ligand-1 (PD-L1) immunotherapy and was recently approved by FDA (Aktas et al., 2019). Nab-paclitaxel+anti-PD-L1 combinatorial immunotherapy orchestrates both immune independent and immune dependent anti-tumor responses respectively (Pardoll, 2012), while nab-paclitaxel+anti-DR5 therapy lacks immune activating component. Given that TNBC, ovarian and other solid tumors carry elevated PD-L1 levels and considering the lack of immune activating function in combinatorial nabpaclitaxel+DR5 agonist therapy, here we sought to test the hypothesis if PD-L1 mediated immune evasion potentially contributes to lower anti-tumor response of DR5 agonist antibodies. Using various clinical DR5 agonist antibodies, multiple tumor cell lines and immune sufficient tumor models, here we demonstrate an unexpected PD-L1 cellular and surface stabilization mechanism which is regulated by DR5 agonist activated Rho associated kinase-1 (ROCK1) and proteasome function downstream of death inducing signaling complex (DISC).

### Materials and Methods for the Examples

Mouse strains. 6 to 8 weeks-old (Age), 20-25-gram (Weight), both male and female (Sex) mice were used for tumor xenografts generation, in vivo efficacy studies, imaging studies, TIL isolation studies as described herein. The following mouse strains were employed: C56BL/6 (The Jackson Laboratory, Bar Harbor, Maine, United States of America) Balb/C (The Jackson Laboratory), immunodeficient Balb/c derived athymic Nude Foxn1^{nu}/Foxn1⁺ (Envigo, Indianapolis, Indiana, United States of America), and NOD.Cg Prkdc^{scid}Il2rg^{tm1Wj1}/SzJ also called as NSG mice. All animal procedures were conducted in accordance with approved protocols and in conformity with the relevant regulatory standards of the Institutional Animal Care and Use Committee (IACUC) of the University of Virginia (Charlottesville, Virginia, United States of America) and the Animal Care and Use Review Office (ACURO) of the United States Department of Defense.

Cell lines. The cell lines employed herein are provided in Table 6. All the cell lines were maintained in DMEM, MEM, RPMI-1640, or other required optimal medium supplemented with 10% heat-inactivated fetal bovine serum (FBS), 2 mM glutamine, 100 U/ml penicillin, and 100 g/ml streptomycin (complete medium) unless otherwise specified and as described in Shivange et al., 2018. MC38 cells (provided by S. Ostrand-Rosenberg, University of Maryland, College Park, Maryland, United States of America) were cultured in DMEM supplemented with 10% (vol/vol) FCS and 1 mM penicillin/streptomycin. Patient derived cells lines were maintained in 20% FBS and 100 mM sodium pyruvate in RPMI 1640 media supplemented with GIBCO™ GLUTAMAX™ brand supplement (ThermoFisher Scientific, Waltham, Massachusetts, United States of America) and 1% penicillin/streptomycin (GIBCO™, ThermoFisher Scientific). Various cell lines were trypsinized and expanded as follow: after digestion, the cell suspension was neutralized with complete media and centrifuged 5 minutes at 1500 rpm. The cell pellets were suspended in relevant DMEM/RPMI media and either expanded or seeded after counting using COUNTESS® II brand automated cell counter (Life Technologies, a part of ThermoFisher Scientific). Passaged cell lines were routinely tested for mycoplasma using MYCOALERT™ brand Detection Kit (Lonza Group, Basel, Switzerland).

Recombinant antibody Cloning. The amino acids sequence of various clinical DR5 agonist antibodies are summarized in Table 7. All clinical antibodies were clones and expressed as described in Shivange et al., 2018. Antibodies were engineered by genetically linking variable regions of farletuzumab (Anti-FOLR1 antibody) and lexatumumab (Anti-TRAIL-R2/DR5 antibody) into a human IgG1 framework. The DNA sequences were retrieved from the publically available sources and synthesized as gene string using Invitrogen GeneArt gene synthesis services (ThermoFisher Scientific). After PCR amplification, DNA was gel purified and inserted into a pCDNA 3.1 vector operably linked to a CMV promoter by making use of IN-FUSION® HD Cloning Kit (Takara Bio USA, Inc., Mountain View, California, United States of America). EcoR1 and HindIII digested vector was incubated with overlapping PCR fragments (of various different recombinant DNAs; see list of clones in Table 5 with infusion enzyme (1:2, vector: insert ratio) at 55° C. for 30 minutes, followed by additional 30 minutes incubation on ice after adding E. coli STELLAR™ cells (Clontech Laboratories, Mountain View, California, United States of America). Transformation and bacterial screening was carried out using standard cloning methods. Positive clones were sequenced confirmed in a 3-tier method. Confirmed bacterial colonies were Sanger sequencing upon PCR followed by re-sequencing of mini-prep DNA extracted from the positive colonies. Finally, maxiprep were re-sequenced prior to each transfection. Recombinant antibodies were also re-confirmed by ELISA and flow cytometry surface binding studies as described in Shivange et al., 2018.

Recombinant antibody expression. Free style CHO—S cells (Invitrogen; ThermoFisher Scientific) were cultured and maintained according to the supplier's recommendations. and as previously described (see Durocher & Butler, 2009; Shivange et al., 2018). A ratio of 1:2 (light chain, V_{L}: heavy chain, VH) DNA was transfected using 1 μg/ml polyethyleniamine (PEI). After transfection cells were kept at 37° C. for 24 hours. After 24 hours, transfected cells were shifted to 32° C. to slow down the growth for 9 additional days. Cells were routinely feed (every 2nd day) with 1:1 ratio of Tryptone feed and CHO Feed B (see Table 5). After 10 days, supernatant from cultures was harvested and antibodies were purified using protein-A affinity columns. Various recombinant antibodies employed herein and recombinant target antigens were engineered, expressed, and purified as described in Shivange et al., 2018. Recombinant human Apo2L/TRAIL was obtained from R&D Systems (Minneapolis, Minnesota, United States of America). His-tag Apo2L was also expressed and purified using nickel NTA columns (see Table 5) using standard BL21 bacterial expression system. His-Apo2L that was generated was confirmed alongside a commercial Apo2L using multiple cancer lines. Similarly, the activity of commercial MD5-1 antibody was compared next to a recombinant MD5-1 that was generated using multiple cancer cell lines as described in Shivange et al., 2018.

Antibody purification. Various transfected monospecific and bi-specific antibodies disclosed herein were affinity purified using HITRAP® MABSELECT SURE™ (Catalog No. 11003493; GE Healthcare Life Sciences, Pascataway, New Jersey, United States of America) protein-A columns. Transfected cultures were harvested after 10 days and filtered through 0.2-micron PES membrane filters (Millipore Express Plus, Millipore Sigma, Burlington, Massachusetts, United States of America). Cleaning-in-place (CIP) was performed for each column using 0.2 M NaOH wash (20 mintes). Following cleaning, columns were washed 3-times with Binding Buffer (20 mM sodium phosphate, 0.15 M NaCl, pH 7.2). Filtered supernatant containing recombinant antibodies or antigens were passed through the columns at 4° C. Prior to elution in 0.1 M sodium citrate, pH 3.0-3.6, the columns were washed 3 times with binding buffer (pH 7.0). The pH of eluted antibodies was immediately neutralized using sodium acetate (3M, pH 9.0). After protein measurements at 280 nm, antibodies were dialyzed in PBS using SLIDE-A-LYZER™ 3.5K (ThermoFisher Scientific). Antibodies were run on gel filtration columns as described below to analyze the percent monomers. Whenever necessary a second step size exclusion chromatography (SEC) was performed. Recombinants IgG4-Fc tagged extracellular domain antigens such as rFOLR1, rDR5 etc. were also similarly harvested and purified using protein-A columns.

Size exclusion chromatography. The percent monomer of purified antibodies was determined by size exclusion chromatography. 0.1 mg of purified antibody was injected into the AKTA protein purification system (GE Healthcare Life Sciences) and protein fractions were separated using a Superdex 200 10/300 column (GE Healthcare Life Sciences) with 50 mM Tris (pH 7.5) and 150 mM NaCl. The elution profile was exported as a Microsoft Excel file and chromatogram was developed. The protein sizes were determined by comparing the elution profile with the gel filtration standard (BioRad 151-1901; Hong et al., 2012). Any protein peak observed in void fraction was considered as antibody aggregate. The area under the curve was calculated for each peak and a relative percent monomer fraction was determined as described in Shivange et al., 2018.

Binding studies by ELISA. Binding specificity and affinity of various described IgG1 subclasses were determined by ELISA using the recombinant extracellular domain of DR5/TRAIL-R2. For coating 96-well ELISA plates (Olympus), the protein solutions (2 μg/ml) were prepared in coating buffer (100 mM sodium bicarbonate pH 9.2) and 100 μl was distributed in each well. The plates were then incubated overnight at 4° C. Next day, the unbound areas were blocked by cell culture media containing 10% FBS, 1% BSA and 0.5% sodium azide for 2 hours at room temperature. The serial dilutions of antibodies (2-fold dilution from 50 nM to 0.048 nM) were prepared in blocking solution and incubated in target protein coated plates for 1 hour at 37° C. After washing with PBS solution containing 0.1% Tween-20, the plates were incubated for 1 hour with horseradish peroxidase-(HRP) conjugated anti-human IgG1 (ThermoFisher Scientific). Detection was performed using a two-component peroxidase substrate kit (BD Biosciences, San Jose, California, United States of America) and the reaction was stopped with the addition of 2N sulfuric acid. Absorbance at 450 nm was immediately recorded using a Synergy Spectrophotometer (BioTek Instruments, Winooski, Vermont, United States of America), and background absorbance from negative control samples was subtracted. The antibody affinities (Kd) were calculated by non-linear regression analysis using GraphPad Prism software.

In Vitro Cell Viability Assays. Cell viability following Apo2L, lexatumumab, KMTR2, tigatuzumab, AMG-655, MD5-1, avelu-MD5, etc. treatments as indicated in the Figures either alone in combination with inhibitors (ROCK1, ERK, caspase inhibitors, etc.) or in combination with an anti-Fc reagent (For tigatuzumab, AMG-655, MD5-1, etc.) were determined using the ALAMARBLUE™ cell viability assays and MTT cell proliferation assays as per the manufacturer's protocols (ThermoFisher Scientific). Briefly, cells were treated with increasing concentration of various antibodies as indicated along with relevant positive and negative control antibodies for 6 hours, 24 hours, or 48 hours as indicated. For each cell killing assay the figures show the representative profiles from n=2-4 with different cultured confluency. Whenever used for immunoblotting, following antibodies treatment caspase-3 processing in tumor cells was monitored using selective antibodies that recognize cleaved human caspase-3 or total caspase-3 (Catalog Nos. 9661 and 9668; Cell Signaling Technology, Danvers, Massachusetts, United States of America). TRAIL-R2 receptor in oligomerization was determined using immunoblotting assays (Cell Signaling Technology Rabbit mAb, Catalog No. 8074). Cell viability was additionally examined by flow cytometry based apoptotic detection methods using 7-aminoactinomycin D (7-ADD) exclusion from live cells.

IC50 Determination. IC₅₀ values were calculated using MTT assays. Cells were seeded in 96 well plates. The next day, after cultures had become adherent, cells were incubated for 48 hours at 37° C./5% CO₂ with the increasing concentrations of the antibodies or drug (such as cisplatin) as indicated. Before treatments, various antibodies were dialyzed into PBS and typically had a pH around 7.5. Values obtained after reading the 96 well plates were normalized to IgG control antibody control and IC₅₀ values were calculated using nonlinear dose-response regression curve fits using GraphPad Prism software. The final results shown in the histograms were obtained from three independent experiments. Whenever provided in the curves, the error bars show ±SEM.

Western Blotting. Cells were cultured overnight in tissue culture-treated 6-well plates prior to treatment. After antibody treatment for 48 hours or another indicated time, cells were rinsed with PBS and then lysed with RIPA buffer supplemented with protease inhibitor cocktail (ThermoFisher Scientific). Centrifugation at 14000 rpm for 30 minutes cleared lysates and protein was quantified by PIERCE™ BCA Protein Assay Kit (ThermoFisher Scientific). Western blotting was performed using the Bio-Rad SDS-PAGE Gel system. Briefly, 30 μg of protein was resolved on 10% Bis-Tris gels and then transferred onto PVDF membrane. Membranes were blocked for one hour at room temperature in TBS+0.1% Tween (TBST) with 5% non-fat dry milk. Membranes were probed overnight at 4° C. with primary antibodies. Membranes were washed three times in TBST and then incubated with anti-rabbit or anti-mouse secondary antibodies (1/10,000 dilution, coupled to peroxidase) for 1 hour at room temperature. Membranes were then washed three times with TBST and immunocomplexes were detected with SUPERSIGNAL™ West Pico Chemiluminescent Substrate (ThermoFisher Scientific). Images were taken using a Bio-Rad Gel Doc Imager system. Primary antibodies are listed in Table 5.

Pre-neutralization assays. Whenever indicated throughout the manuscript text or in figure legends, variable domain pre-neutralization of DR5 agonist antibodies or TNFα antibody was carried out. For in vitro and in vivo studies indicated antibodies and indicated recombinant antigens (rDR5 etc.) were incubated together (either 1:1 or 1:5 ratio, as indicated) at 37° C. for 1 hour with shaking on a platform. As a control, indicated non-preneutralized antibodies were also incubated at 37° C. for 1 hour shaking on a platform either with PBS alone or with recombinant non-specific proteins such as rHER2 or rGFP. Following pre-neutralization antibodies were either used in vitro for cell killing assays, or for cellular/tumor lysates generation (immunoblotting), or for live in vivo live imaging etc. as indicated.

Flow cytometry. The cell surface expression of PD-L1, CD47, huDR5, muDR5, CD8, CD4, CD25 etc. was analyzed by flow cytometry. Overnight grown tumors were trypsinized and suspended in FACS buffer (PBS containing 2% FBS). Single cell suspensions were then incubated with primary DR4/DR5 antibodies for 1 hour at 4° C. with gentle mixing. Following wash with FACS buffer, the cells were then incubated with fluorescently labeled anti-Rabbit antibody for 1 hour. Cells were washed and flow cytometry was performed using a BD FACSCALIBUR™ flow cytometer. The data was analyzed by FCS Express (De Novo Software, Los Angeles, California, United States of America) and FlowJo.

Generation of DR5 Resistant cell lines. DR5 resistant TNBC and ovarian cell lines were generated in manner similar to that described in Wu et al, 2005. Anti-DR5 antibody resistance variants MDA-MB-436 and OVCAR3 of each cell line were derived from each original cell line by continuous exposure to antibodies following initial dose-response studies of KMTR2 and Lexatumumab (0.1 nM-100 nM) over 90 days. Cell viability assays were carried every week to test the resistance. Initially, each cell line was treated with 1 nM of lexatumumab for 72 hours. The media was removed, and cells were allowed to recover for a further 48 hours. Then, next round of treatment was carried out after doubling the previous treatment dose. The dose incremental analysis was carried out for approximately 3 months for each cell line, during which IC₅₀ concentrations were re-assessed in each resistant line. Cells were then maintained continuously in the presence of lexatumumab at these new IC₅₀ concentrations for a further 2 months. After 3 months, resistance was confirmed using multiple DR5a agonists. Using this procedure, stable anti-DR5 resistant lines were generated for OVCAR3, MDA-MB-436, MDA-MB-231, etc.

Epithelial to Mesenchymal (EMT) transition of tumor cells. EMT in indicated tumor cell lines (A549, OVCAR-3, etc) was induced as described in Asiedu et al, 2011. Briefly, cells were treated with recombinant growth factors such HGF (c-Met), TNFα, and TGFα. Either Fc-conjugated or commercial c-Met, TNFα and TGFβ were added to epithelial cell cultures A549 and OVCAR-3 with concentration of 50 ng/ml c-Met, 200 ng/ml of TNFα along with 60 ng/ml of TGFβ. These concentrations were standardized in lab after testing 20-200 ng/ml of TNFα and 1-10 ng/ml of TGFβ by keeping c-Met concentration constant. Treatment was carried out for a period of 4-6 days even after splitting the cells. Expression of E-cadherin, N-cadherin, vimention, etc. was analyzed after 24, 48, 72, and 96 hours of treatment.

Mechanical dissociation of tumors to obtain single-cell suspensions. Viable single cells from tumor tissues were isolated as described in Leelatian et al, 2017. Briefly, after indicated antibody treatments (4-6 doses) mice were euthanized and tumors were harvested using sterile scissors and forceps. After excision of tumor, they were minced into small pieces in sterile RPMI-1640 media using two single-edged razor blades. Small tumor pieces were passed through a 70 m cell strainer in sterile RPMI-1640 media. A rubber plunger and syringe were used to mesh the dissociated cells through the cell strainer and media containing dissociated cells was collected onto a sterile labeled conical tube. Dissociated tumor cells were subjected to flow cytometry (FACS) analysis for PD-L1, DR5, N-cadherin, FOLR1, CD47 etc. surface expression as described under flow cytometry protocol.

PD-L1 surface expression analysis of tumor derived cells. The cell surface expression of PD-L1 from tumor-derived cell was analyzed by flow cytometry. Isolated cells were suspended in FACS buffer (PBS containing 1% FBS). The single cell suspension was then incubated with primary PD-L1 antibodies (1:400 dilution) for 1 hour at 4° C. with gentle mixing. We confirmed surface PD-L1 with commercially available antibodies as well as with clinical antibodies avelumab and atezolizumab (Zhang et al, 2017). Following 3 times wash with FACS buffer, the cells were then incubated with fluorescently labeled (alexa 488) anti-Rabbit secondary antibody for 1 hour. Cells were then washed 3-4 times with FACS buffer and flow cytometry was performed using BD FACSCALIBUR™. The data was analyzed by FCS Express (De Novo Software) and FlowJo software.

HPG incorporation and analysis. HPG incorporation was essentially carried out as described in Calve et al, 2016. HPG (Click Chemistry Tools, Scottsdale, Arizona, United States of America) were diluted in PBS, raised to pH 7.4 with NaOH, sterilized with a 0.22 m filter and stored at −20° C. Methonine free RPMI media was purchased and was added to 100 nM final concentration of HPG. Cells were grown either in regular RPMI (called Met+media) or in Methonine free RPMI with HPG (called Met-HPG+media). HPG incorporated proteins within the lysates were labeled selectively with copper-catalyzed AF555-conjugated alkyne or azide using CuAAC, which results in a stable triazole adduct. The CLICK-IT® HPG Alexa Fluor 488 Protein Synthesis Assay Kit was used for flow cytometry as per the manufacturer's protocol. Similar to ³⁵S-methionine, CLICK-IT® HPG was added to cultured cells and the amino acid is incorporated into proteins during active protein synthesis. Detection of the incorporated amino acid in cells for flow cytometry studies (following addition of exosomes or EVs) utilizes a chemoselective ligation or click reaction between an azide and alkyne, where the alkyne-modified protein is detected with either Alexa Fluor 488 or Alexa Fluor 594 azide. Labelled cells were analyzed for flow cytometry as described herein.

Exosomes and Apoptotic cell derived EVs isolation. Exosomes were isolated as described in Shen et al, 2011. For apoptotic cell derived extracellular microvesicles (EV) isolation, clarified tissue culture supernatant was spun three times at 20,000×g for 30 mins and was used. Although we only showed the data with apoptotic cell derived EVs, similar trends in surface presence of PD-L1 were observed when purified exosome were added to DR5Ko cells. For each trial, 6×10⁶ cells were seeded onto 2×150 mm dishes in a total volume of 60 ml of DMEM supplemented with 10% exosome-free FCS and grown for 72 hours. For all exosome studies, the tissue culture media was spun at 5000×g for 15 minutes. The pellet was discarded and the supernatants (SN) were passed through 0.22 um filter. For exosome analysis by SPIRI and IFM, the filtrate was concentrated by angular flow filtration (Centricon Plus-70; EMDMillipore) to a final volume of to 500 l. Exosomes were purified by size exclusion chromatography (Izon qEV column), 500 μl fraction samples were collected, and fractions 4, 5, and 6 were assayed by immunoblot (IB) to confirm the presence of exosome markers, pooled, and interrogated by SPIRI and IFM. For exosome analysis by IB, the clarified tissue culture supernatant was spun twice at 10,000×g for 30 minutes to remove contaminating microvesicles, and the resulting supernatant was spun at 70,000×g for 2 hours at 4° C. to pellet exosomes. Cell lysates were generated by addition of 2 ml of 2×SDS-PAGE sample buffer. Exosome pellets were resuspended in 600 μl of 2×SDS-PAGE sample buffer. Immunoblots were performed at a constant ratio of exosome:cell lysates. IB analysis was performed by separating proteins by SDS-PAGE at a constant ratio of cell and exosome lysates. Proteins were then transferred to Immobilon membranes (EMDMillipore), followed by incubation with block solution (0.2% non-fat dry milk in TBST), primary antibody solution, and secondary antibody solution, with multiple washes in TBST between each step. Antigens were visualized by chemiluminescence and detected using a Amersham Imager 600 (GE Healthcare Life Sciences) gel imaging system. The resulting digitized IB images were then examined in Image J. Each was converted to 8 bit grayscale followed by background subtraction. Measurement parameter and scale were set to integrated density and pixel, respectively. Images were then inverted, bands were delineated using the freehand selection tool, and signal densities were converted to relative protein abundance by multiplying by the dilution factor for each sample. Relative budding was calculated by dividing the relative protein abundance in exosome lysate by the sum of the relative protein abundance in the cell lysate and the relative protein abundance in exosome lysate.

Chimeric DR5 cloning and Human DR5 agonist testing studies in Balb/c animals. Chimeric DR5 that contained extra cellular domain (ECD) of human DR5 and transmembrane (TM) and intracellular domain (ICD) of mouse DR5 were constructed. Two chimeric DR5 constructs were used: (1) without any linker between human ECD and mouse TM called Chi-DR5; and (2) with G4S linker between human ECD and mouse TM called Chi-G4SDR5. These constructs were synthesized using Invitrogen gene synthesis service and cloned in to either EFla-driven viral expression vectors CD550A (pCDH-EF1α-MCS-BGH-PGK-GFP-T2APuro, system biosciences) or in pCDN3.1 vector with G418 selection and direct transfection using restriction-cloning sites (EcoR1-Not1). Viral infection had higher stable generation efficiency or direct lipid-based transfections. Only Chi-G4S-DR5 was expressed on 4T1 and MC38 cell surface as confirmed by FACs. MC38 wild type cells and MC38 Chi-G4SDR5 expressing lines were tested using C57BL/6J mice while 4T1 wild type 4T1 Chi-G4S-DR5 expressing lines were tested using Balb/c mice for syngeneic immune investigations. Since 4T1 is breast tumor lines, 6-8 weeks old female Balb/c mice were injected with 4T1 Chi-G4S-DR5 cells subcutaneously (SC) in their right flank with 0.5×10⁶ cells in matrigel. These cells consistently formed tumors within ˜2 weeks. Tumors were monitored as described herein above. Next, mice bearing ˜100 mm³ tumors were weight matched and randomly assigned into groups and injected with antibodies as described in the text (100 μg of KMTR2 or lexatumumab alone or with 100 μg of avelumab). Similar tumor experiments were carried out using 4T1 WT cells and were treated with MD5-1 alone or MD5 plus avelumab. Antibodies were injected intraperitoneally. After 4-6 doses, tumors were harvested and subjected for tumor-infiltrating lymphocytes (TILs) isolation as described in Tan & Lei, 2019 and herein below.

TIL isolation by Ficoll-Paque density gradient centrifugation. TILs from 4T1 and MC38 tumors were isolated as described in Tan & Lei, 2019. Briefly, after indicated antibody treatments mice were euthanized. Next, tumors were harvested using sterile scissors and forceps. After excision, tumors were minced into small pieces in RPMI-1640 media using two single-edged razor blades. Small tumor pieces were transferred to a 70 m cell strainer in RPMI-1640 media. A rubber plunger of a syringe was used to mesh the dissociated cells through the cell strainer and cloudy media (that contained dissociated cells) was collected onto a sterile labeled 50 ml conical tube. Tubes were filled with 30 ml of RPMI-1640 media at room temperature (18° C.-20° C.). Immediately before the addition of Ficoll-Paque media, single-cell suspension was well mixed with 25 ml pipet. Thoroughly mixed 10 ml of Ficoll-Paque media was carefully poured in the bottom of the tube to form a layer of Ficoll-Paque below the cell suspensions without mixing the cell suspension. Tubes were centrifuged at 1025 g for 20 minutes at 20° C. with slow acceleration and without applying any brake. 20 ml of the upper layer of media was discarded to a waste bottle from the tube. Layer of mononuclear cells that contained TIL was transferred to a sterile labeled 50 ml conical tube using a sterile pipette, along with the remainder of the media above the Ficoll-Paque. TILs were washed three times using 40 ml of complete RPMI media each time. After final wash, isolated TILs were subjected for flow cytometry (FACS) analysis with fluorescently labeled CD4, CD8, CD25, etc. antibodies as described herein and in Whitford et al, 1990.

IHC studies. Chi-G4S-DR5 stable MC38 tumors harboring mice were treated (6 total doses) lexatumumab, avelumab, ROCK1i, lexatumumab+ROCKi, and avelumab+lexatumumab and IgG1 control as indicated. Mouse tumors were collected at 100-200 mm3 & embedded in O.C.T. to make blocks and mouse spleen also collected & embedded as a positive control. Samples were processed and sectioned into 4 m tissue sections. Tumor sections were stained with antibodies (CD8, CD4, Fox3p) and counter-stained with hematoxylin. Peroxidase conjugated anti-rabbit/anti-rat IgG reagents were used as secondary antibody. Reactions were developed using 3,3′-diaminobenzidine (DAB) as chromogenic substrate. Then, slides were dehydrated and mounted. Finally, brightfield images were taken using brightfield microscope. For quantification, 5-6 images were acquired at 20× magnification for each tumor sample.

ApoEV isolation. 4×10⁶ cells were seeded onto 3×150 mm dishes in a total volume of 45 ml of DMEM supplemented with 10% exosome-free FBS and grown for 12 hours. Media of each plate was changed with serum-free medium and incubate the cells at 37° C. for 6-12 hours, with or without various antibody treatments. For all ApoEV studies, the tissue culture media was spun at 8000×g for 15 minutes. The pellet was discarded and the supernatants (SN) were passed through 0.22 um filter. ApoEVs were isolated by the ultracentrifugation method. Collected tissue culture media was spun at 5,000×g for 10 minutes to remove contaminating cellular particles, and the resulting supernatant was spun at 80,000×g for 4 hours at 4° C. to pellet ApoEVs. ApoEV pellets were resuspended in 500 μl of serum-free medium and were used for either cellular treatments, dot blots assays and immunoblotting assays.

PD-1/PD-L1 reporter assay. PD1/PD-L1 reporter assay was carried with some modifications from manufacturer's original assay protocol (Catalog Nos. J1250 and J1255, Promega), similar to what is described in Wang et al, 2017b. The original kit contains PD-1 effector Jurkat T cells stably express human PD-1 and NFAT-induced luciferase, while PD-L1 aAPC/CHO-K1 cells stably express human PD-L1 and a cell surface protein designed to activate cognate TCRs in an antigen-independent manner. Upon PD-1-PD-L1 interaction luciferase signal is downregulated. Antibodies blocking PD-1-PD-L1 interaction removes inhibitory signals, resulting in luciferase activation. Modified assay for tumor-jurkat co-culture studies: MDA-MB-436, MDA-MB-231, A549, Colo-205, HCT-116 and OVCAR3 etc. cells were plated in 96-well cell culture plate at 40,000 cells/well in 100 μl of medium (RPMI/DMEM, 10% FBS, 0.2 mg/ml hygromycin-B) and incubated overnight at 37° C. in 5% CO₂. Next day, medium was removed from the culture plate and cells were treated with diluted DR5 agonist antibodies. Antibodies were diluted in assay buffer containing RPMI-1640 +1% FBS in 40 μl dilution per well, with or without inhibitors and incubated for few hours, as described in the text and figures. Glo response NFAT-luc2/PD1 Jurkat cells (Promega) were re-suspended in assay buffer and incubated with 1 μg/ml anti-CD3 for 2 hours to activate PD1 Jurkat cells. Activated PD1 cells were re-suspended and 40 μl amount of activated cells were added to each test well of 96-well culture plate (that contained DR5 treated tumor cells) at a concentration of 1.25×10⁶/ml. After 6 hours of co-culture, assay plates were removed from the incubator; content of each well were mixed thoroughly with the help of multi-channel pipette and then transferred to a white opaque assay plate. 10 mg/ml luciferase reagent was prepared and loaded into the plate reader, which injects 50 μl reagent in each well and measures luminescence immediately. Data was analyzed using Microsoft Excel software. Wherever indicated, inhibitors (ERK, STAT3, Caspase, ROCK1 etc.) were added 2-3 hrs prior to DR5 agonist antibodies in 96 well culture of tumors as described herein.

Native PD-L1 immunoprecipitation studies with avelumab. Cells were cultured in 10 cm tissue culture dishes for 24 hours prior to treatment. Before treatment, culture medium was replaced with serum free medium. Cells were treated with indicated DR5 agonist antibodies (20-50 nM) as described herein. After 6 hours of antibody treatment, 400 nM of avelumab (human anti-PD-L1 antibody with IgG4-Fc) was added to the culture media of each dish and incubated at 4° C. for 1 hour. Then, cells were harvested and lysed with IP lysis buffer (20 mM Tris pH 7.5, 150 mM NaCl, 1 mM EDTA, 10% Glycerol, 1% Triton-X, 0.5 mM PMSF) supplemented with protease inhibitor cocktail (ThermoFisher Scientific). Spinning at 14000 rpm for 30 min collected clear protein lysates and protein was quantified by Pierce BCA protein assay kit. 1-1.5 mg protein (˜400-500 μl) was taken into Eppendorf microcentrifuge tube. Protein lysates were incubated with anti-human IgG4 specific beads for 2 hours at 4° C. placing into a rotating wheel. Protein conjugated beads were washed thrice with phosphate buffered saline (PBS). Finally, beads were boiled at 100° C. for 5 minutes with 30 μl of SDS sample buffer. 15-20 μl sample was loaded into the SDS-gel and western blotting was performed using the Bio-Rad SDS-PAGE Gel system followed by immunoblotting using indicated PD-L1, CMTM6 and ROCK1 specific antibodies.

Generation of CRISPR DR5 Knockout tumor cells. Using Synthego's Gene Knockout Kit v2, we performed genomic deletion ofDR5 in various tumor cell lines. Briefly, per manufacturer's instructions ribonucleoprotein (RNP) complexes that consist of purified Cas9 nuclease duplexed with chemically modified synthetic guide RNA (sgRNA) targeting DR5 were delivered to the cell lines by using LIPOFECTA^{M} IE™ CRISPRMAX™ Transfection Reagent. DR5 knockout lines were further selected by treatment with 200 nM DR5 agonist antibody, which initiates apoptosis in left over wild-types cells to get a complete DR5-knockout population. Particularly, we first prepared plates by pre-warming 2×24-well cell culture plates with 500 μl of normal growth medium in each well. Next, RNP complexes were assembled in 1.3:1 sgRNA to Cas9 ratio and working concentrations (3 μmol/μl) of RNPs were prepared in a microcentrifuge Tube 1 as shown below:

RNP Preparation (Tube 1)

| Component | Molarity | Volume (per reaction) |
|---|---|---|
| OPTI-MEM ™ I Reduced | — | 25 μl |
| Serum Medium | | |
| sgRNA | 3 μM (pmol/μl) | 1.3 μl (3.9 pmol) |
| Cas9 | 3 μM (pmol/μl) | 1 μl (3 pmol) |
| LIPOFECTAMINE ™ Cas9 | — | 1 μl |
| Plus Reagent | | |
| TOTAL VOLUME | — | 28.3 μl |

Next, transfection solution was prepared in a separate microcentrifuge tube (Tube 2) that contained LIPOFECTAMIN4E™ CRISPRMAX™ reagent in OPTI-MEM™ I reduced serum medium. The concentration was used as described in table below (26.5 μl reaction volume) as per the manufacturer's protocol:

Transfection Solution (Tube 2)

| | Volume (per |
|---|---|
| Reagent | reaction) |
| OPTI-MEM ™ I Reduced Serum Medium | 25 μl |
| LIPOFECTAMINE ™ CRISPRMAX ™ Transfection | 1.5 μl |
| Reagent | |
| TOTAL VOLUME | 26.5 μl |

The transfection solution (Tube 2) was directly added to RNPs mix (Tube 1) and mixed well by pipetting up and down. The mix (˜50 μl) was next incubated for 10 minutes at room temperature followed by addition on freshly trypsinized cells in growing phase 0.42-1.2×10⁵ cells in 500 μl of the growth medium. The mix and cells with 500 μl growth media were distributed into two wells. Media was replaced after 24 hours of incubation and cells were allowed to grow for 2-3 days. The cells on the first plate were lysed and processed to analyze editing efficiency. The cells on the second plate were cultured for use in assays, banking, and/or single-cell cloning.

huDR5, Chi-DR5, Chi-G4S-DR5 stable 4T1 and MC38 stable line generation. Transfection of various DR5 constructs (huDR5, Chi-DR5, Chi-G4S-DR5) into the different cell lines (4T1, MC38, ID8) was achieved byjetOPTIMUS DNA transfection reagent for recombinant DR5 cloned in pCDN3.1 vector. In brief, 60-70% confluent cells were grown in 10 cm culture dish. Mixing 10 g of plasmid DNA and 10 μl of transfection reagent into 1 ml of jetOPTIMUS buffer made transfection solution. After incubating for 10 minutes at room temperature the transfection mix was added on the cells. The cells were further allowed to grow for 24 hours and then selected using 2 mg/ml of G418. Particularly, 10 μg DNA was diluted into 1000 μL jetOPTIMUS buffer and vortexed. This was followed by addition of 10 μL jetOPTIMUS into the DNA solution (ratio 1:1 corresponding to g DNA: L reagent) and vortexed and spun down briefly. Mixture was incubated for 10 minutes at room temperature. Next transfection mix was added drop wise onto the cells in serum containing medium and distribute evenly. Plates were incubated at 37° C. for 24 hours. The next day, transfection medium was replaced with by cell growth medium and cells were allowed to grow for another day before starting G418 2 mg/ml selection. Media was changed every days and reliable GFP signal was evident 72 hours of transfection.

Lentiviral preparation and transduction. Lentiviral packaging and delivery was executed by using the technology from system Biosciences (see chimeric DR5 cloning section herein above) and method was very similar to that described in Wollebo et al, 2013. Briefly, lentivirus was prepared by transfecting 293T cells in T75 flask with transfer vector (6 μg) and packaging vectors (3 μg each) in the ratio of 2:1:1:1 using 30 μg of PEI. The virus containing culture medium was collected 48 and 72 h after transfection, cleared by filtration (0.45 m Millipore, Bedford, Massachusetts, United States of America) and concentrated by 20% PEG 6000. After centrifugation at 3000 g for 30 minutes the pellet was resuspended in 1/10th of the initial volume in phosphate-buffered saline (PBS)/0.1% bovine serum albumin (BSA), stored at −70° C. For transduction the 60-70% confluent cells were plated in 10 cm plate and 5 ml virus along with 5 μg/ml polybrene was added. Transduction Medium was replaced with growth medium after 12 hours and allowed the cells to grow for another 24 hours. The transduced DR5 positive cells were selected using 2.5 μg/ml puromycin. In details, HEK 293T cells were cultured in high glucose-containing Dulbecco's modified Eagle's medium (DMEM) supplemented with 10% fetal bovine serum (FBS) at 37° C. in 5% CO₂. For transfection the cells were seeded a day before at a density of 70-80% in 10 cm culture dish. Transfection mix was prepared as following: Transfer plasmid with gene of interest (6 μg), pVSVG plasmid (3 μg), pREV (3 μg), pRRE (3 μg), OptiMEM media (500 μl) and PEI (30 μg). Transfection mixture was vortex mixed and quick centrifuged and incubated at room temperature for 10 minutes. Transfection mixture was then added gently on the cells through the wall and mix by tilting the plate. Transfected cells were incubated at 37° C. for 12-16 hours and medium was replaced with 10 ml of fresh growth medium. Virus containing culture media was collected after 48 hours and 72 hours of incubation. The floating cell debris were separated by quick spin at 1000 rpm for 5 minutes and then virus containing culture medium was filtered through a 0.45 m filter (Millipore). Next virus was concentrated by using PEGylation in the following ratio: Virus suspension (40 ml), 50% PEG 6000 (10 ml) and 5 M NaCl (1 ml). PEGylated solution was mixed and incubated at 4° C. overnight on gentle rocker. The precipitated virus particles were centrifuged at 4000 rpm for 30 minutes and resuspended in 4 ml of culture medium. To transduce, the tumors cells were seeded a day before at a density of 60-70% in 10 cm culture plate. Transduction solution was prepared by mixing 2 ml of virus, 5 μg/ml of polybrene, and 1x HEPES buffer and gently added on the cells with 8 ml of culture medium. Cells were allowed to grow at 37° C. for an additional 12 hours and then medium was replaced with growth medium. The 2.5 μg/ml puromycin selection was performed after 48 hours of transduction and medium was replaced each day with intermediate PBS washing to avoid the accumulation of dying cell debris. Stable cells appeared within a week.

Nude and syngeneic tumor xenograft studies. All animal procedures were conducted under the accordance of University of Virginia Institutional Animal Care and Use Committee (IACUC) and (DoD ACURO) approved protocols and conform to the relevant regulatory standards. Details of mice strains, age and sex used are provided above. Briefly, 6 to 8 weeks-old (Age), 20-25-gram (Weight), both male and female (Sex) mice were used for tumor xenografts generation, in vivo efficacy studies, imaging studies, TIL isolation studies as described herein. The mouse mice strains that were used are described herein above. Various different solid cancer cell lines were used for tumor nude xenograft studies as described in text. Weight and age (6-8 weeks old) matched mice were injected subcutaneously (SC) in their right flank with indicated cell lines in matrigel. Different cell number was injected as some cells were highly effective and some required higher density during xenografts. Tumor cells were mixed 100 μl volume with matrigel. For antitumor efficacy studies, mice bearing ˜100 mm³ tumors weight matched animals were randomly assigned into groups and injected (either 25 μg or indicated different dose) either intraperitoneally or intravenously (as indicated) three times per week with the indicated antibodies. Tumors were measured in two dimensions using a caliper as described previously (Wilson et al, 2011; Graves et al, 2014). Tumor volume was calculated using the formula: V=0.5axb², where a and b are the long and the short diameters of the tumor respectively. (n=4-6 animals were used for each therapeutic antibody injection). The p-values are determined by two-tailed paired Wilcoxon Mann-Whitney test. For syngeneic and surrogate tumor xenograft studies, most experiments were carried out using TNBC 4T1 cell lines. MC38, a colon cancer line forms tumor in C57BL/6J, while 4T1 was only effective in forming tumors in Balb/c immune sufficient mice strain. Similar to nude xenograft studies, 6-8-week-old female littermate of matched size and weight Balb/c mice were injected subcutaneously (SC) in their right flank with 0.5×106 4T1 cells lines in matrigel. 4T1 cells very highly consistently in forming tumors (19 out of 20) within ˜2-3 weeks as described in Takeda et al, 2008. For tumor regression studies and TIL isolation studies mice bearing ˜100 mm³ tumors were (after matching tumor size, n=4-7) randomly assigned into groups and injected with therapeutic antibodies as indicated (50-100 μg dose) intraperitoneally two-three times per week. For surrogate efficacy studies, MD5-1, MD5-1+avelumab and IgG1 control were engineered with IgG1 KO-Fc and S267E mutations. Avelumab was engineered as IgG4-Fc. Tumors were measured two-three times a week and volumes were calculated as the product of three orthogonal diameters similar to nude animal studies as described in previous section. The p values are determined by two-tailed paired Wilcoxon Mann-Whitney test. For Biochemical analysis, signal cell isolation or TIL of tumors, mice were euthanized after indicated antibody treatment followed by tumor extraction (see Wilson et al., 2011; Li & Ravetch, 2012).

Quantitation and statistical analysis. Data unless indicated otherwise are presented SD. In general, when technical replicates were shown for in vitro experiments, student t-test was used for statistical analysis and the same experiment was at least repeated once with similar trend observed. When data from multiple experiments was merged into one figure, statistical significance was determined by either unpaired t test, paired t test or Wilcoxon Mann-Whitney test etc as indicated under Data information at the end of figure legends using Graph Pad Prism 5.0 software. Tumor growth curves are displayed as mean±SEM. For all the statistical experiments p values, p<0.05 (*), p<0.005 (**) and p<0.0001 (***) were considered statistically different or specific p values indicated otherwise or “ns” indicates non-significant.

### Example 1

### PD-L1 Stabilization by DR5 Agonist Antibodies in Solid Tumors

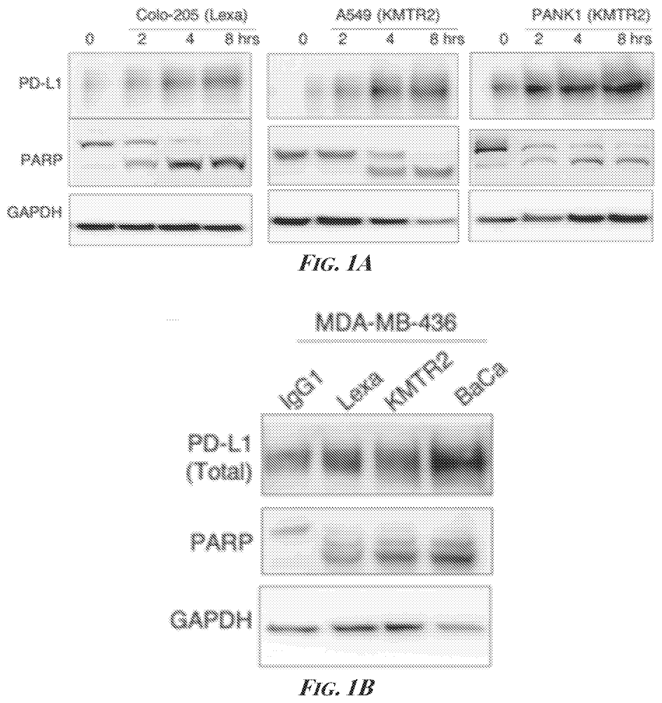

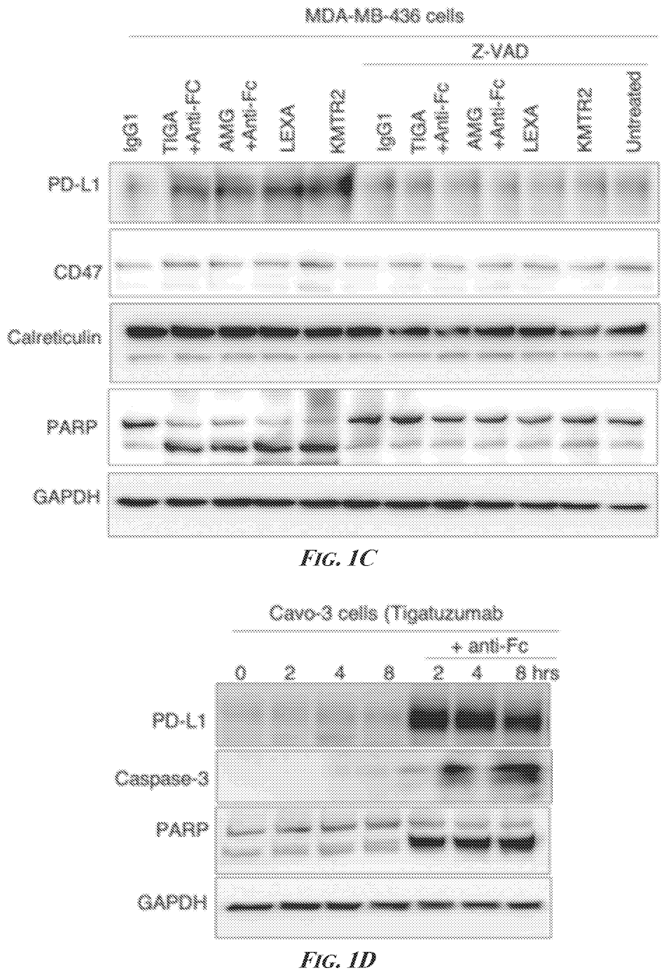

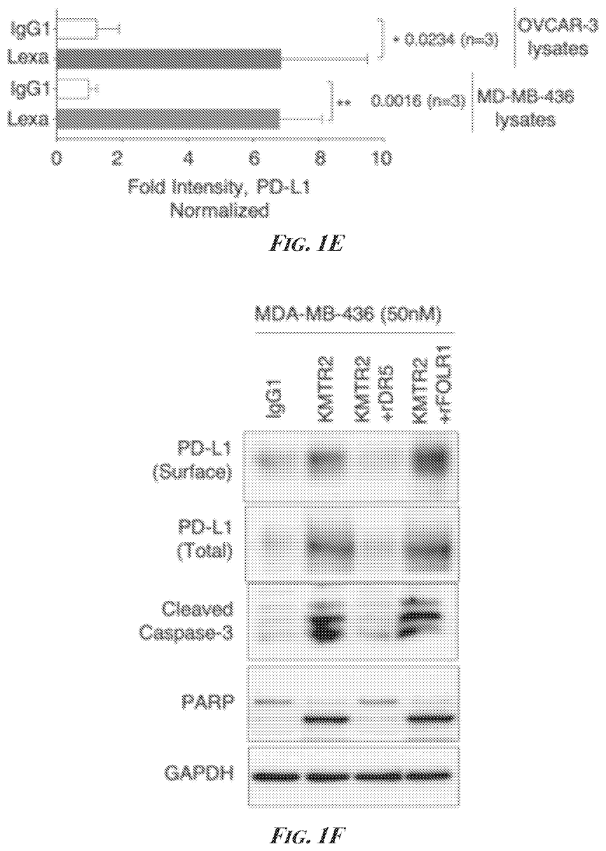

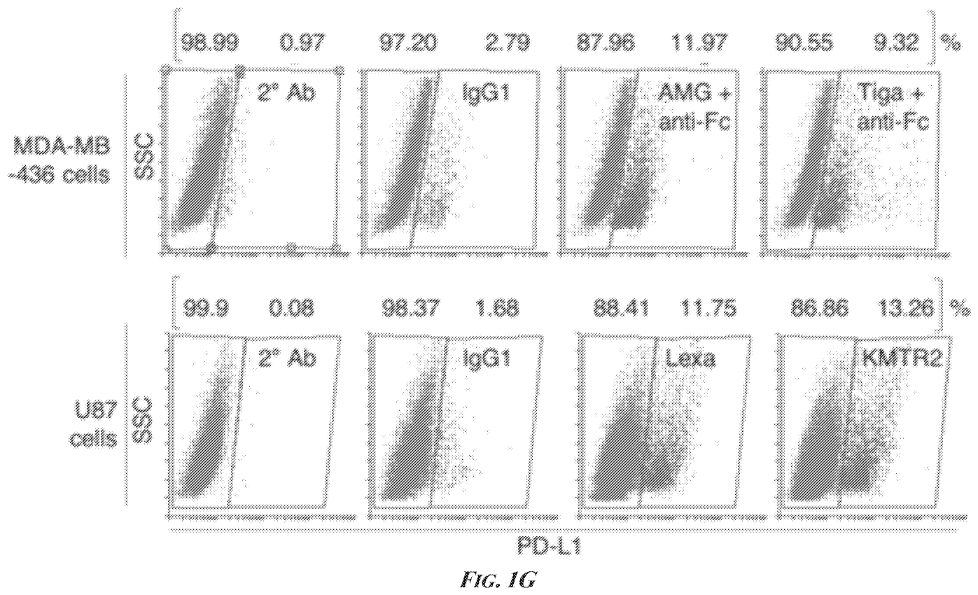

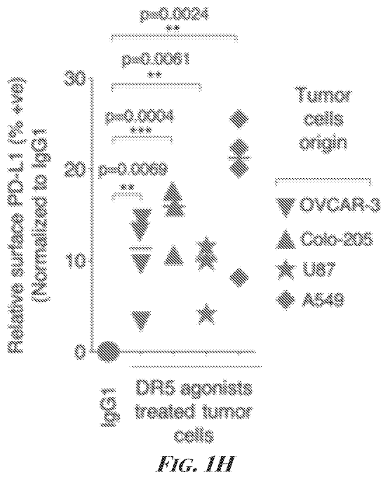

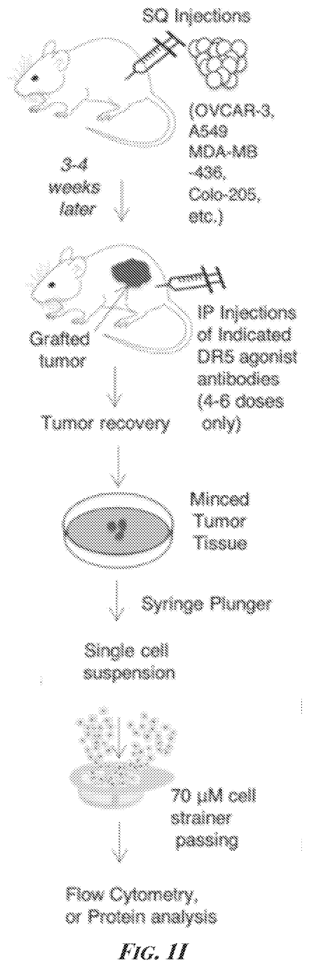

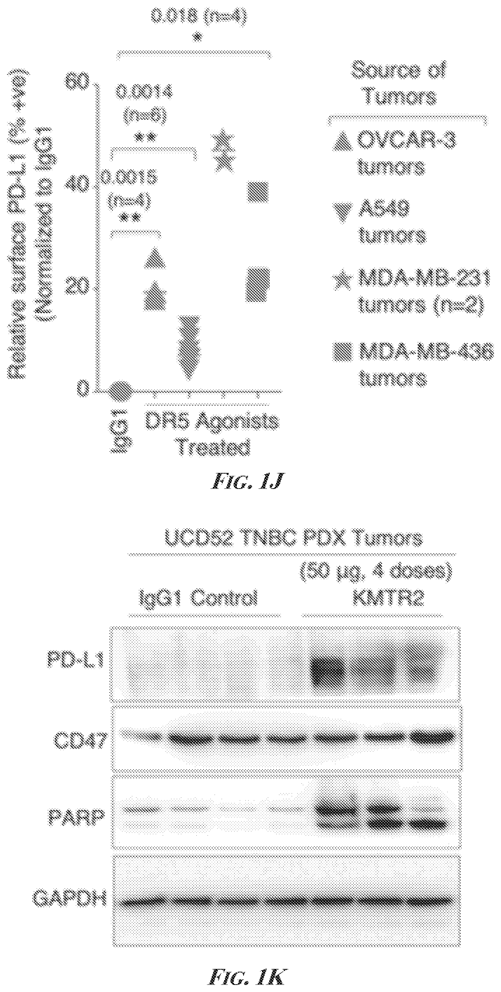

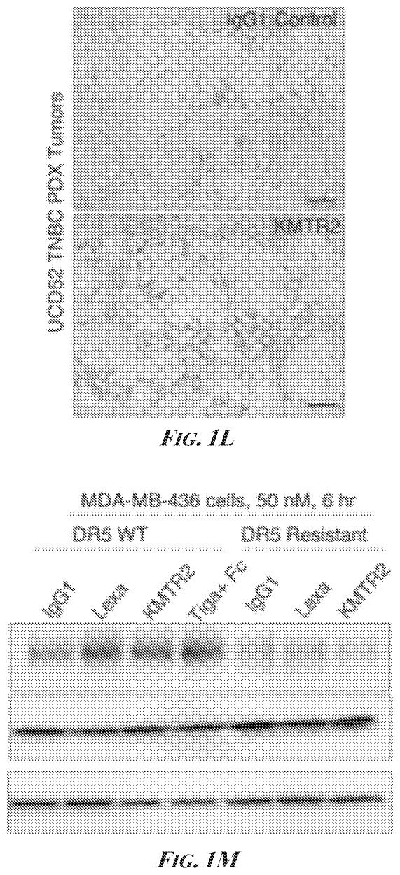

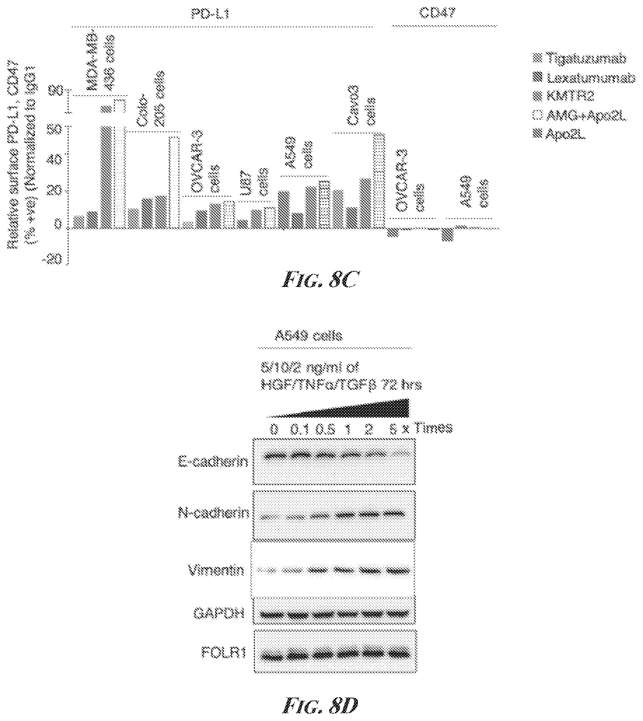

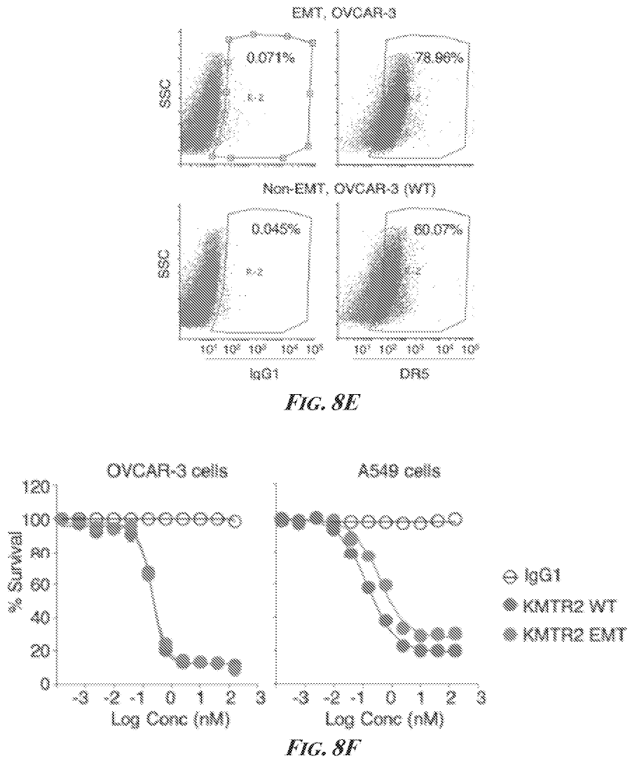

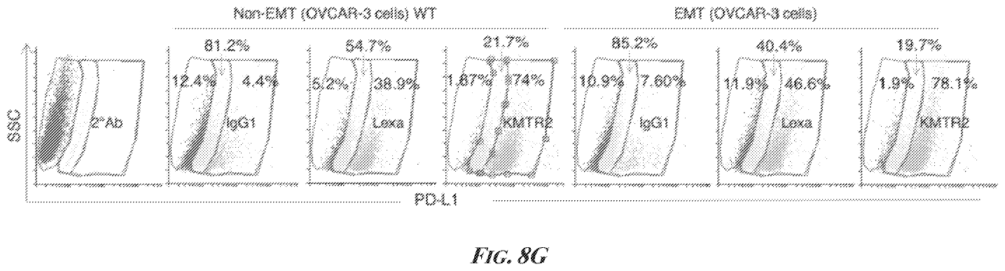

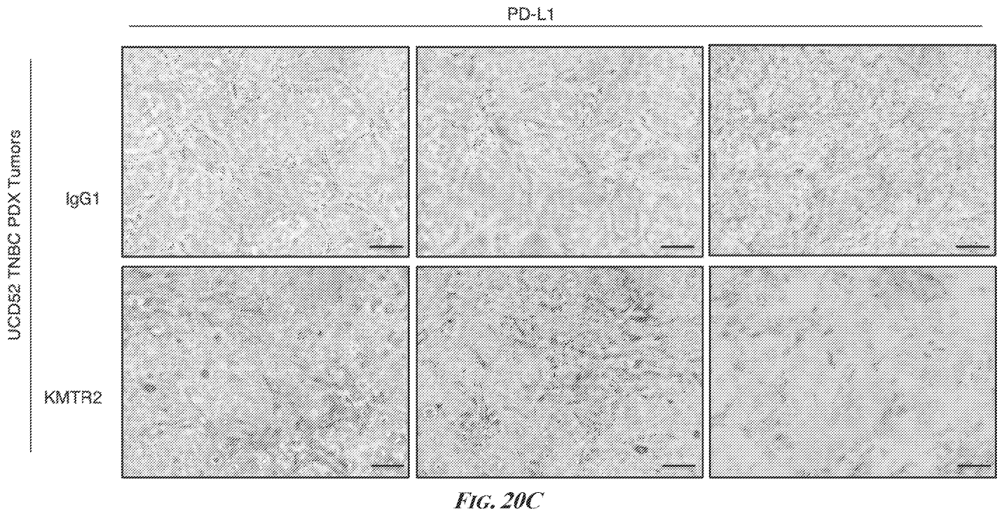

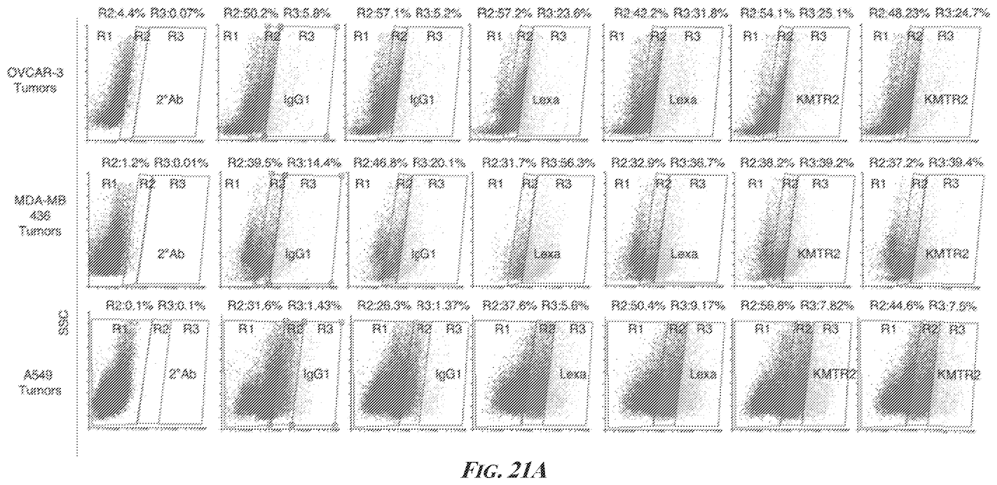

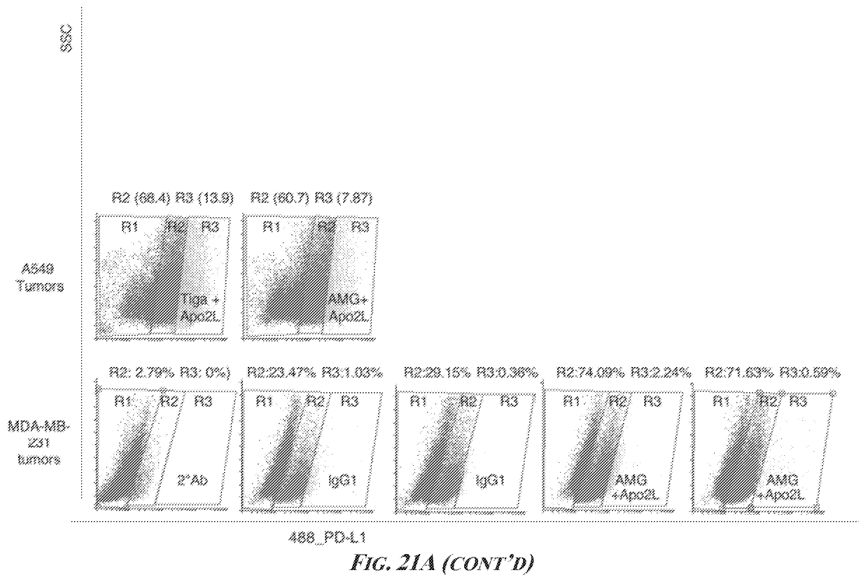

In last decade, multiple ligand-receptor interactions on immune cells-tumor cells (vice-versa) called immune checkpoints (ICPs), have accounted for T-cell dysfunction/exhaustion in various tumor types (Pardoll, 2012; Vonderheide, 2015). Humanized antibodies blocking these ICP interactions are highly compelling in generating curative immune response in clinical trials (Mellman et al., 2011; Pardoll, 2012). One key ICP ligand called PD-L1, present on many solid tumor cell types, inhibits activity of antigen specific CD8+ T-cell via interacting with PD-1. This results in evasion and immune dysfunction in solid tumors. Given the elevated function of PD-L1 in solid tumors, we tested its expression in response to DR5 signaling upon agonist antibodies treatment. Unexpectedly, we observed PD-L1 stabilization in cellular lysates by various clinical DR5 agonists in tested solid cancer cell lines (see FIGS. 1A-1E, 8A, 8B, and 20A). Blockade of caspase-8 containing death inducing signaling complex (DISC), downstream of DR5 inhibited PD-L1 stabilization (FIGS. 1C and 8A). Importantly, cellular lysate stabilized PD-L1 was recruited and increased on surface of various tumor cells as confirmed by surface biotinylating assays (FIGS.s 1F and 8B) and flow cytometry (FIGS. 1G, 1H, 8C, 19A, and 19B). We observed increase in both mean fluorescent intensity (MFI) of PD-L1 and the relative % of cells expressing PD-L1 after various DR5 agonists treatment (FIG. 20B).

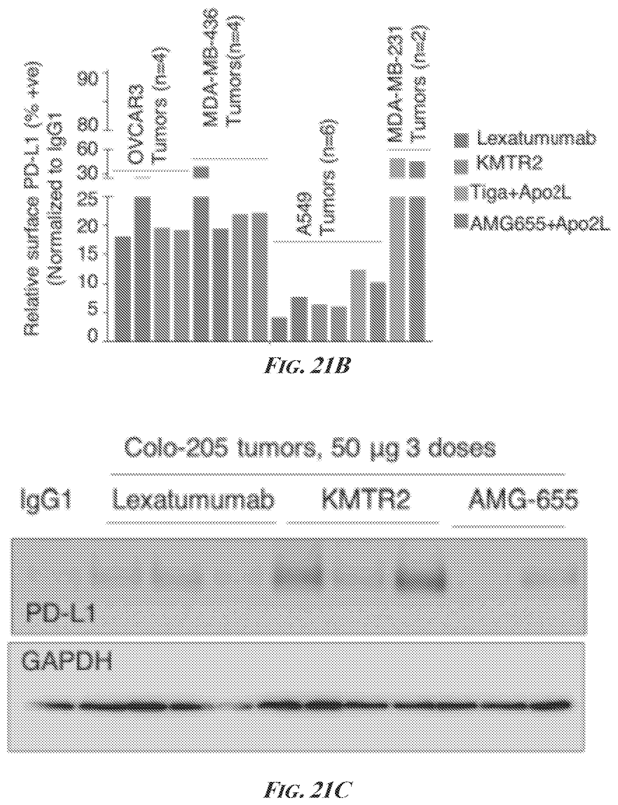

Next, we analyzed total and surface PD-L1 stabilization in vivo using cellular and patient derived solid tumor xenografts treated with DR5 antibodies. All DR5 agonist antibodies were engineered with CH₂ Fc L234A L235A (LALA) mutations to avoid interference from compliment system and NK cell mediated antibody dependent cellular cytotoxicity (Saunders, 2019). Isolated single cell suspension of tumor cells (FIG. 1I) from TNBC, ovarian, lung, and colon tumor cellular and TNBC PDX xenografts showed higher total and surface PD-L1 expression from animals treated with DR5 agonist antibodies as compared to IgG1 control (see FIGS. 1J, 1K, and 21A-21C). PD-L1 stabilization was also confirmed via IHC analysis using TNBC UCD52 PDX tumors treated with KMTR2 (FIGS. 1L and 20C).

Given the inverse relation of tumor metastasis and patient survival, we next tested if DR5 antibodies mediated PD-L1 stability would be lost during transient epithelial to mesenchymal transitions (EMT). To this end, we induced transient EMT using A549 (and OVCAR-3 cells) as evident with loss of E-cadherin and gain of N-cadherin and vimentin (FIG. 8D). DR5 expression, sensitivity to DR5 agonists, and PD-L1 surface stabilization remained unaffected in these metastatic transformed cells (FIGS. 8E-8G).

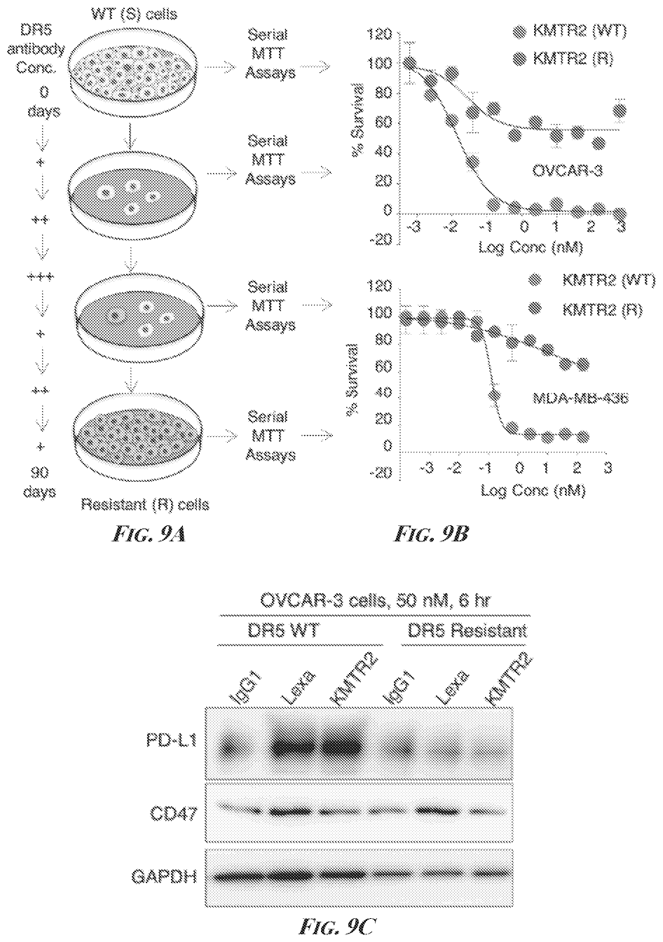

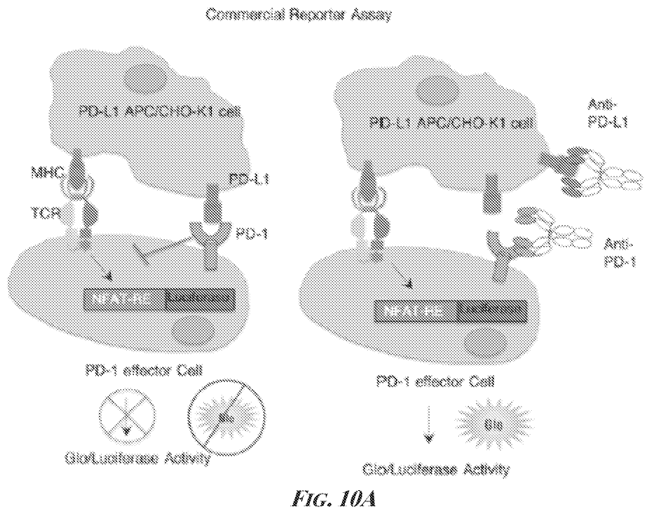

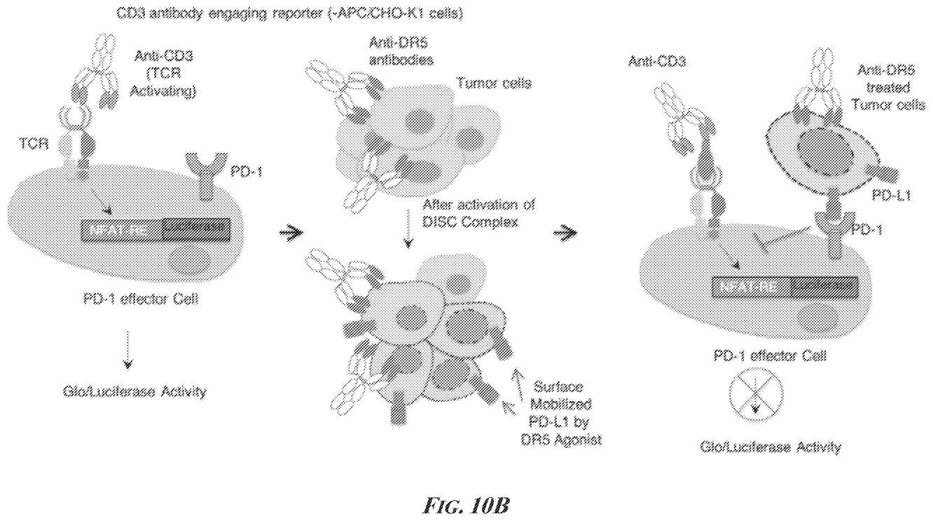

Next, we generated DR5 resistant ovarian and TNBC cell lines via continuous exposure with the increasing concentration of lexatumumab for a period of 3 months (FIGS. 9A and 9B). PD-L1 stabilization was not observed in ovarian and TNBC cells resistant to DR5 signaling (FIGS. 1M and 9C).

### Example 2

### CSN5 is not Required for PD-L1 Upregulation by DR5 Agonist Antibodies

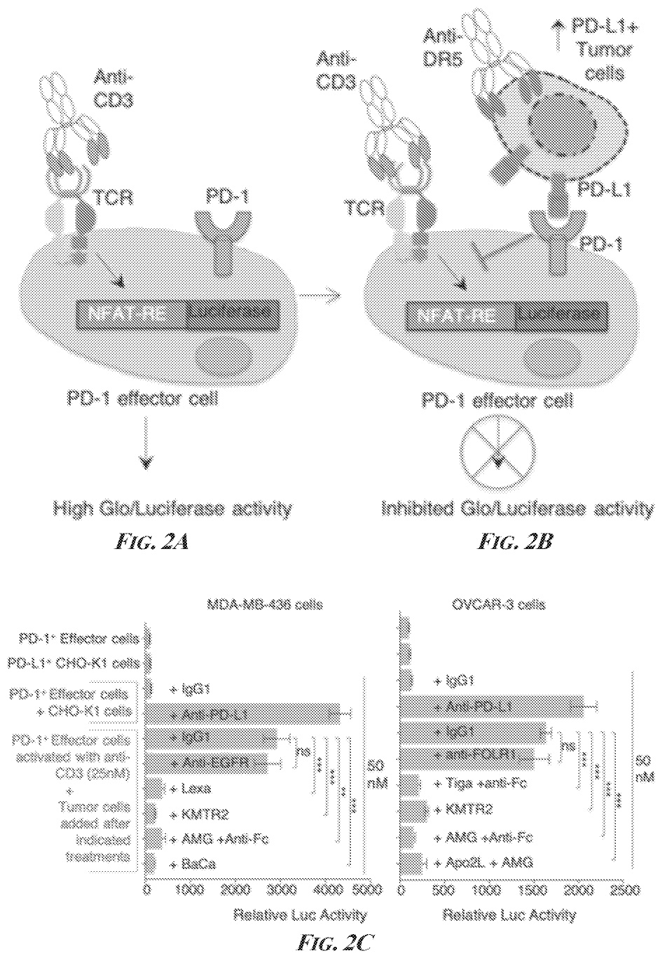

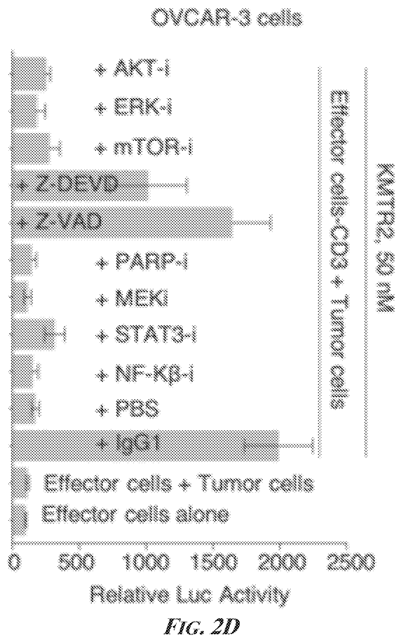

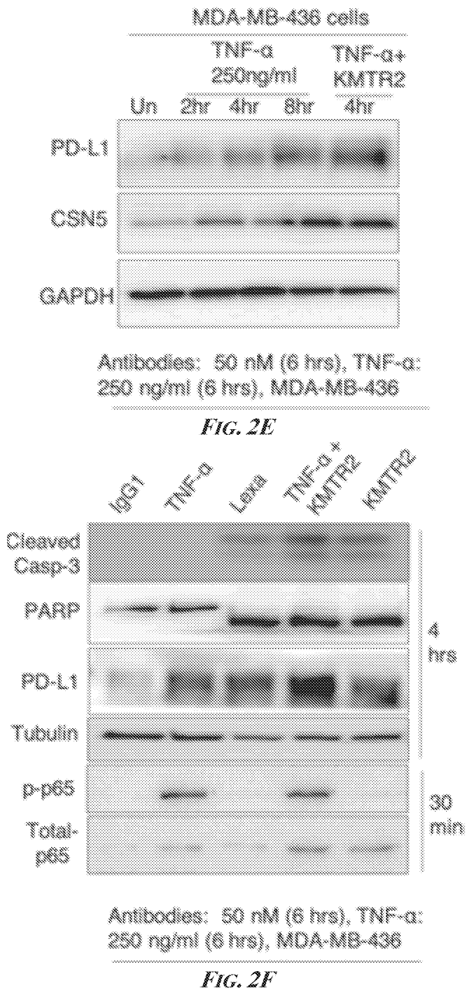

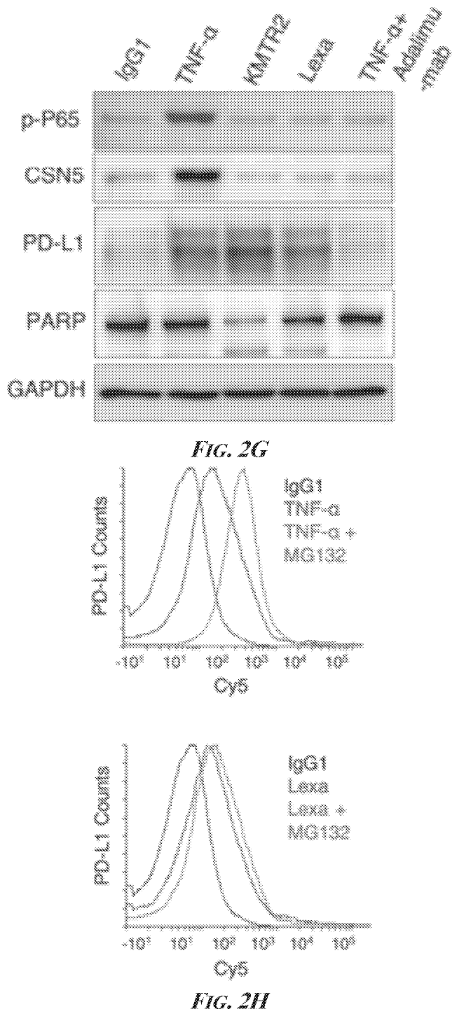

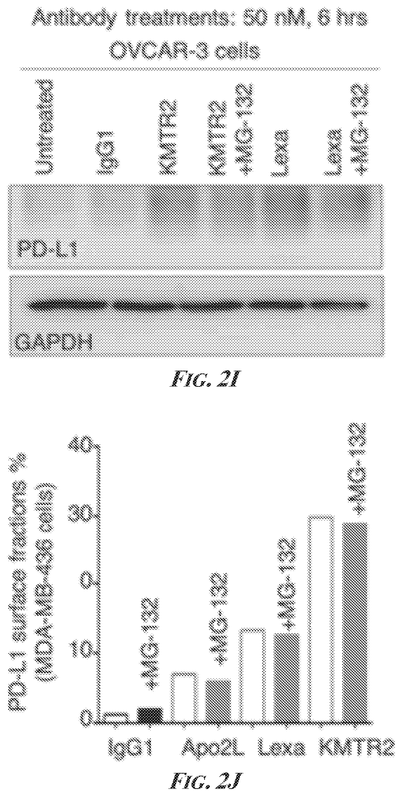

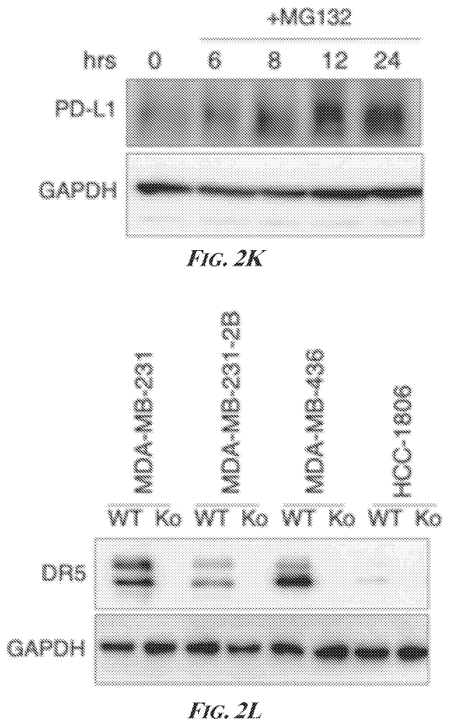

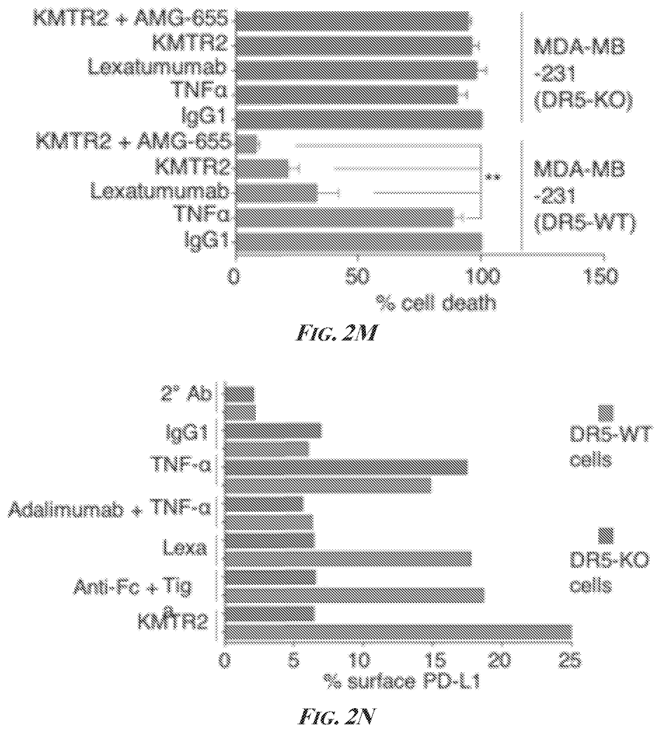

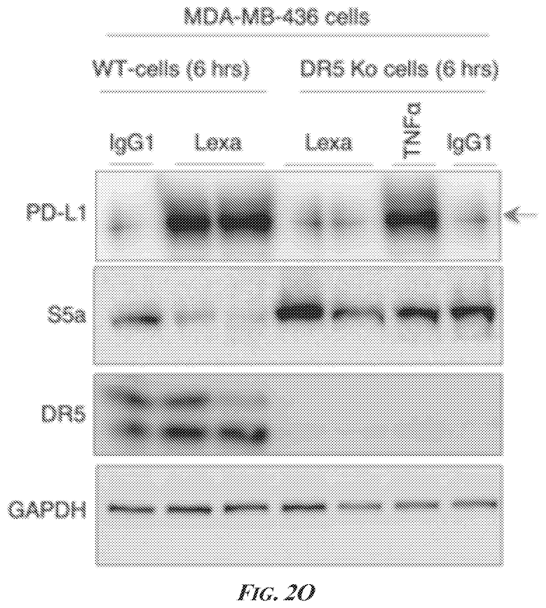

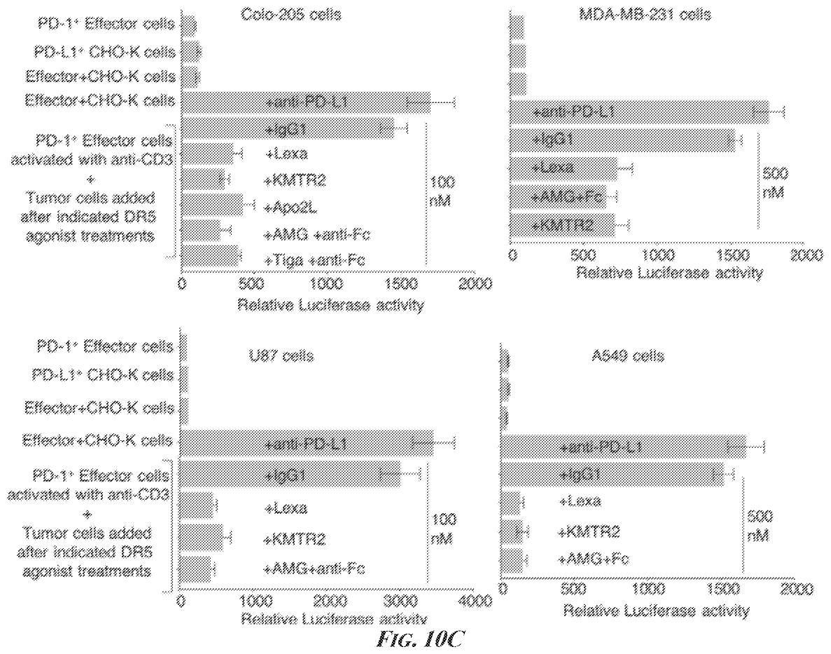

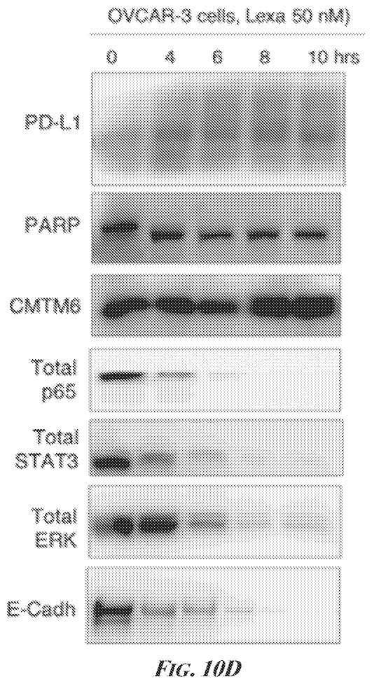

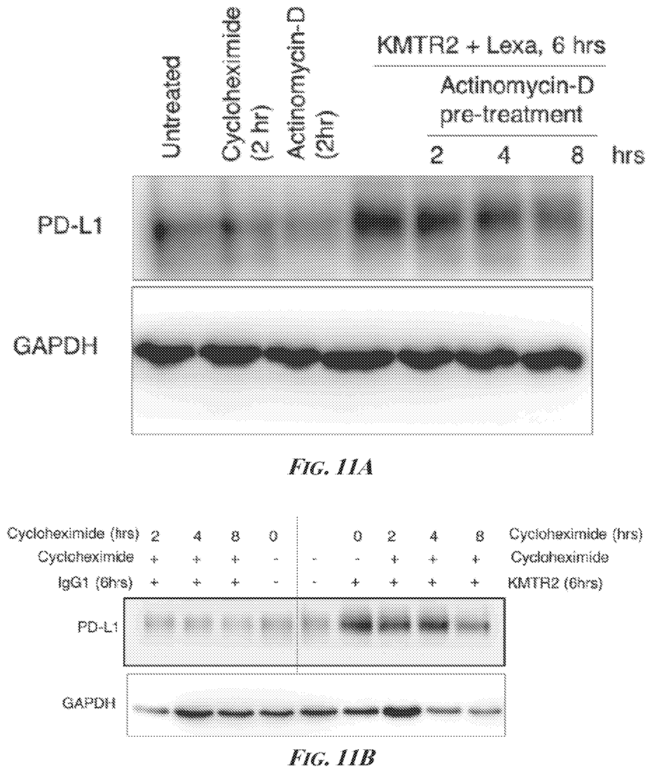

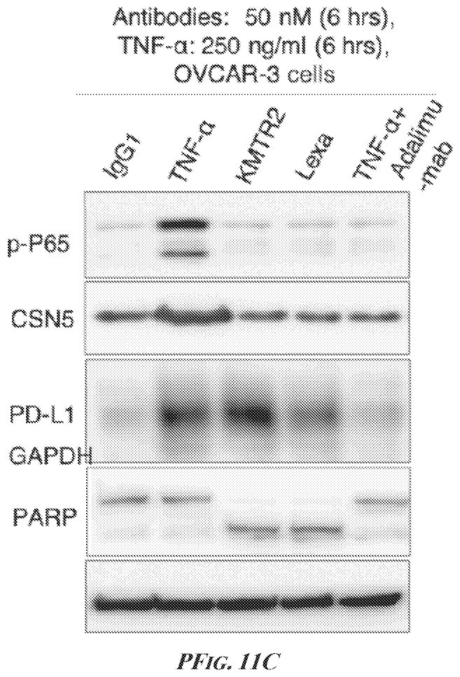

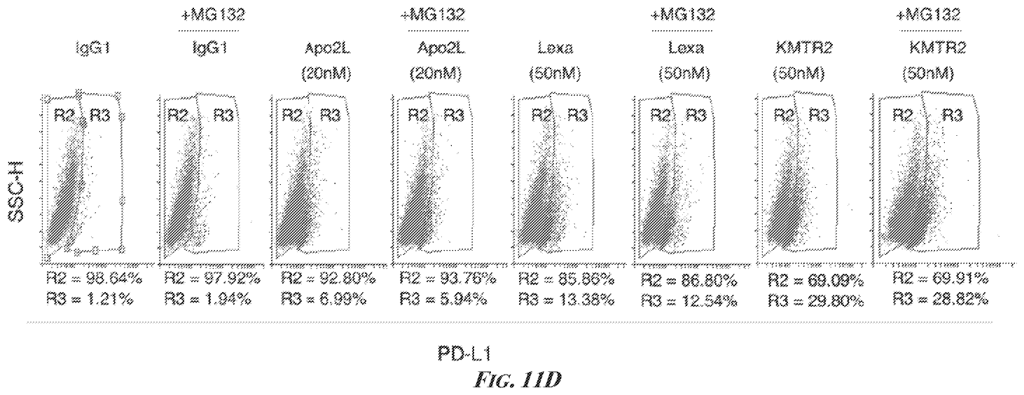

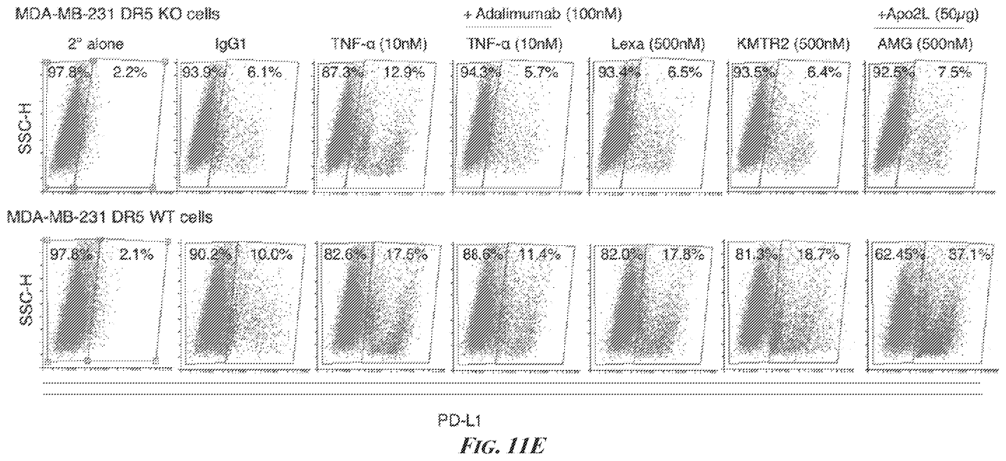

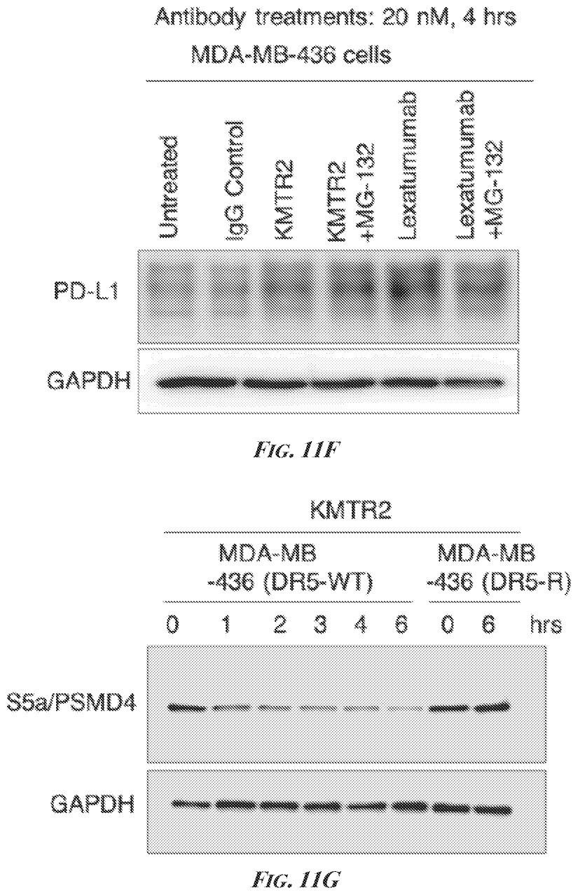

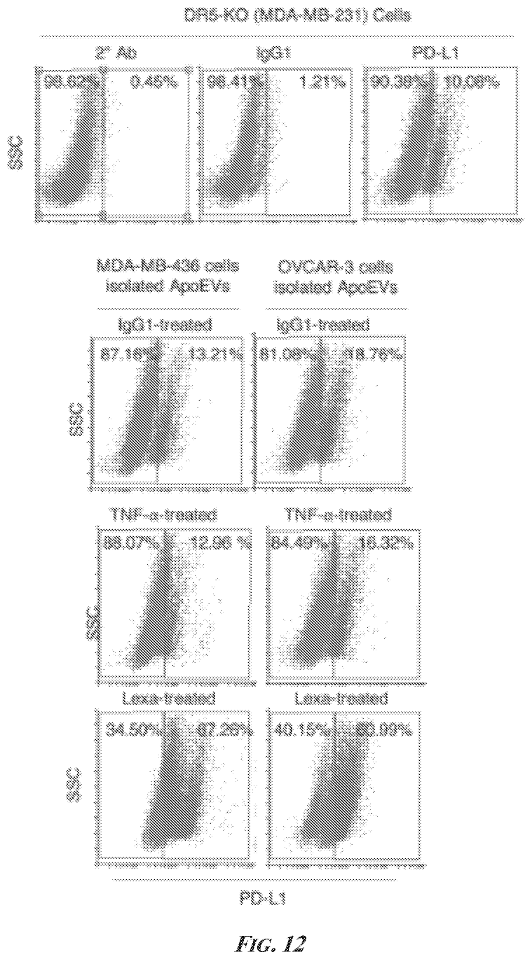

To test whether surface stabilized PD-L1 (by DR5 antibodies) generated an immunosuppressive function, we made use of PD-1+ jurkat cells stably expressing luciferase reporter under NFAT response element (FIGS. 2A and 10A). Treatment with anti-CD3 (OKT3) antibody induced luciferase activity in these reporter cells (FIGS. 2A-2D).

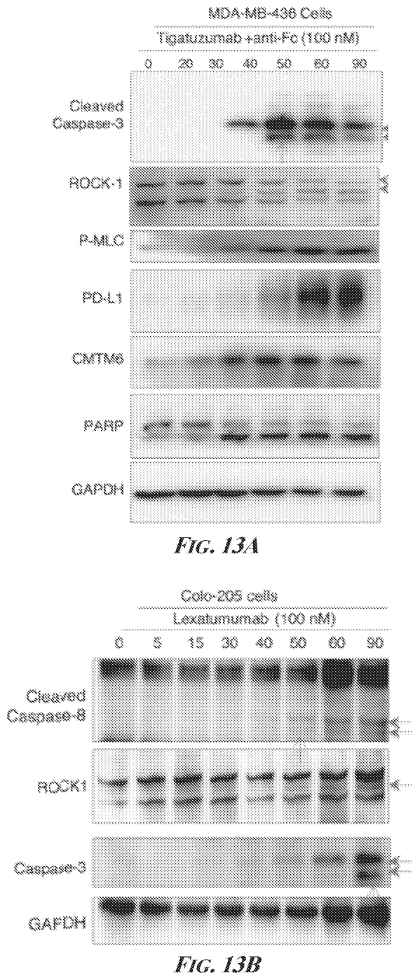

To confirm if tumor cell surface mobilized PD-L1 formed complex with PD-1 checkpoint receptor, jurkat cells were co-cultured with DR5 agonist treated tumor cells (FIGS. 2B-2D, 10B, and 10C). DR5 agonist treated ovarian, TNBC, lung and colon cancer cell lines showed significantly reduced luciferase activity (FIGS. 2C and 10C), confirming PD-L1-PD-1 interaction, a perquisite for T-cell dysfunction and immunosuppression. Strikingly, PD-L1 along with CMTM6 (Burr et al., 2017) a newly identified PD-L1 regulator were stable in tumor cell lysates for significantly longer hours (FIG. D). When tested in DR5 treated tumor cell-jurkat cell co-culture reporter assays, inhibition of various posttranslational PDL1 stability regulators (Hsu et al., 2018) such as mTOR, STAT3, CDK1, and NFkβ did not change PD-L1 surface expression (FIG. 2D). In addition, both transcription inhibitor (actinomysin-D) and translation inhibitor (Cycloheximide) did not also reversed PD-L1 stability generated by DR5 agonists when compared with untreated or IgG1 control cells (FIGS. 11A and 11B). Similar to cellular lysate stabilization (FIG. 1C), inhibition of DISC-caspase-8 activity reduced PD-L1 activity in reporter assays (FIG. 2D). Taken together, these results indicated that PD-L1 stabilization (after DR5 agonist treatments) requires the function of a regulatory pathway, which potentially is not modulated by transcription or translation function rather by the DISC and caspase activation.

Since DR5 ligand Apo2L belong to TNF superfamily and TNF-α was recently shown to stabilize PD-L1 by activating a deubiquitinase COP9 signalosome 5 (CSN5) enzyme (Lim et al., 2016), we tested if DR5 mediated PD-L1 stabilization also requires CSN5. TNBC tumor cells were treated with DR5 agonist antibodies and TNF-α next to each other. TNF-α stabilized both PD-L1 and CSN5 in ovarian and TNBC tumor cells without activating caspases (FIGS. 2E-2G and 11C). CSN5 up-regulation was not detected in DR5 agonist treated lysates (FIGS. 2E-2G and 11C). Mechanistically, CSN5 is a deubiquitinase and functions by removing ubiquitin tags from PD-L1. Thus, CSN5 inhibits PD-L1 degradation by proteasome complex (Lim et al., 2016). Similar to published report (Lim et al., 2016), we also observed increased PD-L1 basal stability after TNF-α and a proteasome inhibitor (MG132) co-treatment (FIG. 2H, top). Strikingly, proteasome inhibition (for a longer period) increased overall PD-L1 in tumor cells lysates (FIG. 2K) and DR5 agonist plus MG132 cotreatment did not additionally stabilize PD-L1 on cell surface or in total lysates (FIGS. 2H-2J, 11D, and 11F). These results indicated that proteasome inhibition (by MG132) potentially is a linear and downstream event of DR5 agonist signaling. As DR5 agonist antibodies functions via direct caspase-8 activation in DISC and given the reports of caspase mediated proteasome inactivation (Cohen, 2005), we next explored the possibility that PDL1 stabilization is a byproduct of proteasome inactivation caspases.

To this end, we generated DR5 knockout (DR5-KO) TNBC cells using CRISPR-Cas-9 approach (FIG. 2L). Generated DR5-KO lines were not sensitive to DR5 agonists (FIG. 2M) and did not mobilize PD-L1 on tumor cell surface (FIGS. 2N and 11E). It must be noted that TNF-α mediated PD-L1 stabilization remained unchanged in DR5-KO lines as compared to DR5-WT cells (FIGS. 2M, 2N, and 11E). If DR5 agonist stabilize PD-L1 via proteasome inactivation, we hypothesized significantly higher degradation of proteasome regulatory submits in DR5-WT tumor cell lines as compared to DR5-KO cells upon DR5 agonist treatments.

Since various proteasome regulatory subunits of 26S proteasome (such as S6′, Si, and S5a/PSMD4) are known to be cleaved during proteasome inactivation (Cohen, 2005; Sun et al., 2004), we next tested total S5a/PSMD4 levels after DR5 agonist treatments in WT and DR5-KO cells. As expected, the proteasome's regulatory submit S5a/PSMD4 was only degraded in MDA-MB-436 WT cells but not in DR5-KO cells (or DR5 resistant cells) after DR5 agonist treatment (FIGS. 2O, 2P (top lane), and 11G). Furthermore, TNF-α signaling stabilized PD-L1 without affecting S5a levels (compare FIG. 2O lane 6 vs. lanes 2, 3). Considering S5a degradation by DR5 agonists represents interference with proteasome function, overall ubiquitin signal increased only in DR5 sensitive cell lysates but not in DR5-KO cells after indicated antibody (Lexa or KMTR2) treatments (FIG. 2P). Collectively these results confirm two different proteasome interference mechanisms of PD-L1 stabilization, where one works by deubiquitinating PDL1 (Lim et al., 2016), while other works by degrading proteasome subunits.

### Example 3

### Role of ApoEVs in PD-L1 Stabilization in Heterogenous Tumors

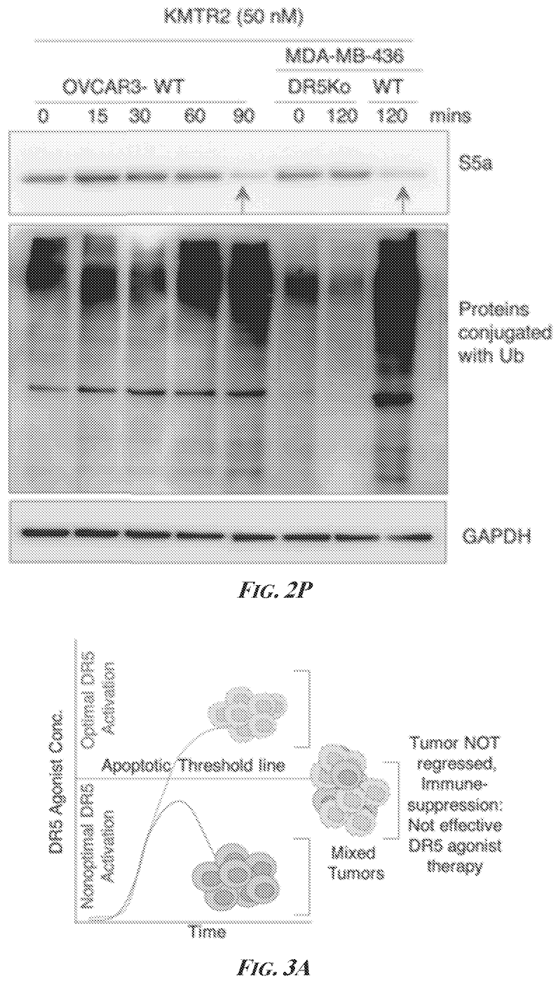

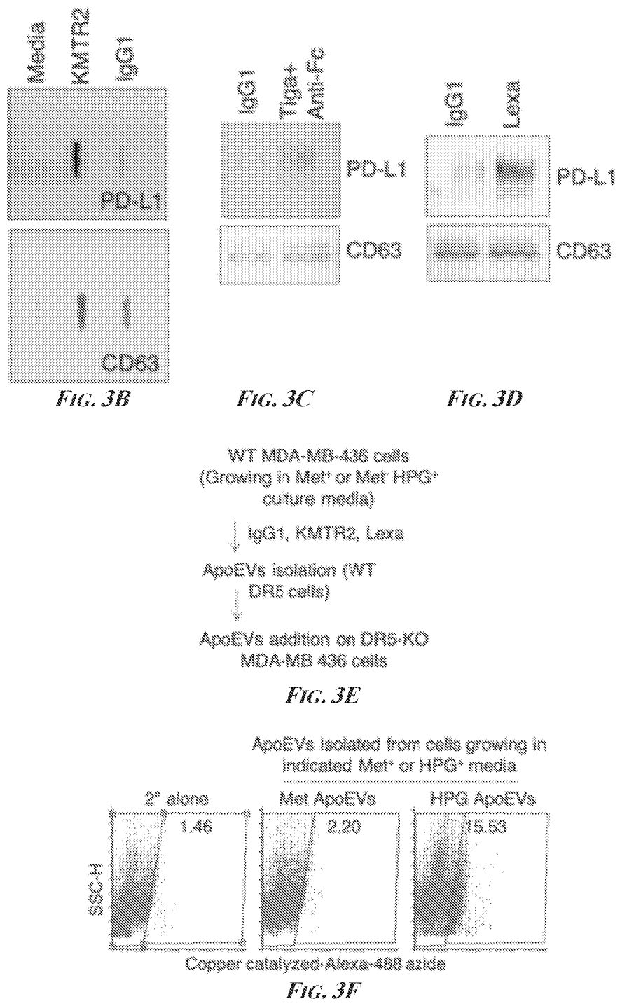

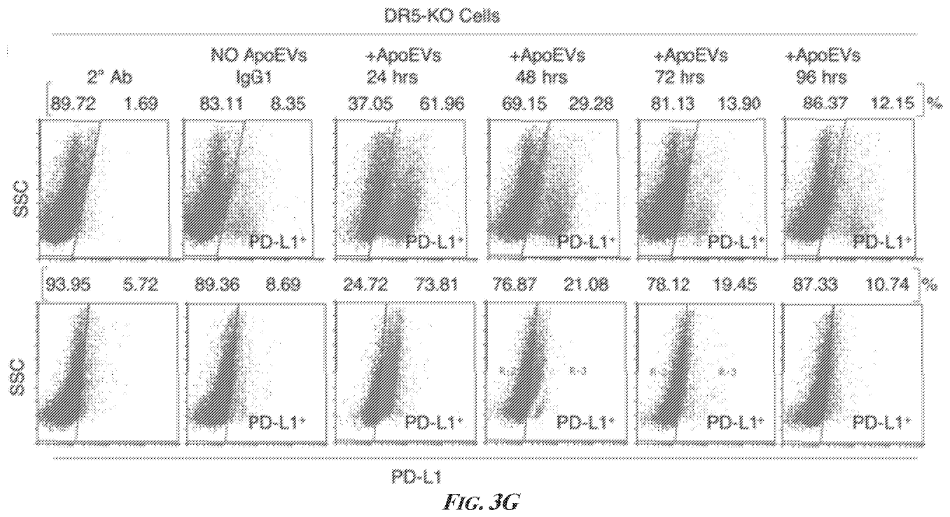

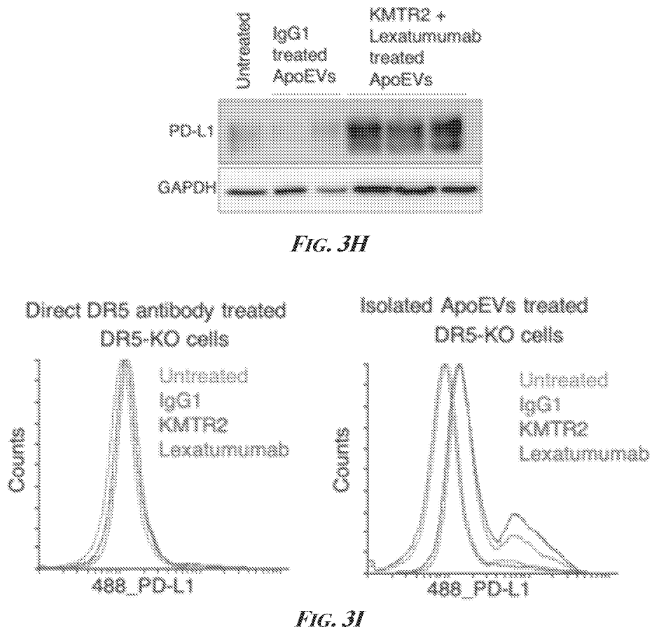

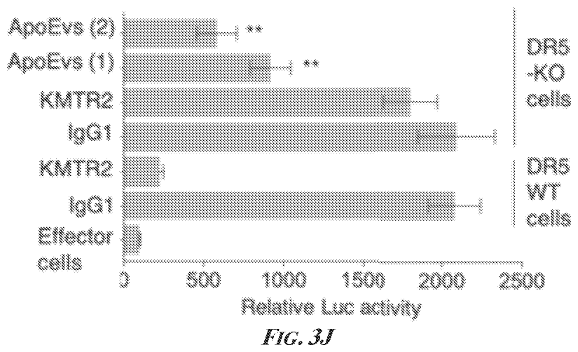

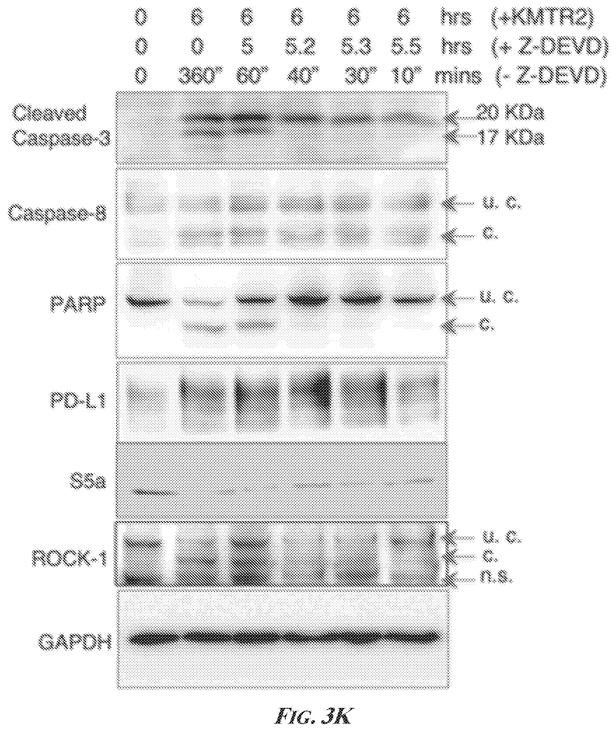

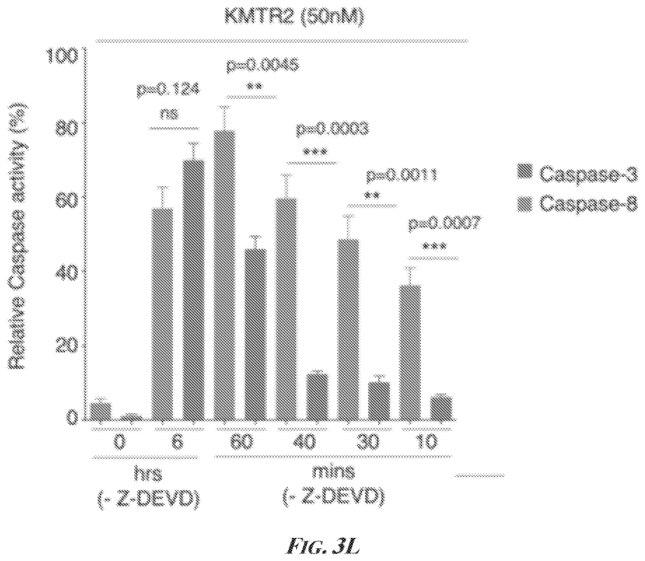

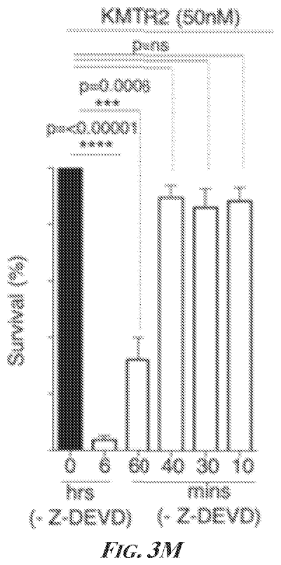

The results presented in FIGS. 1 and 2 suggested that targeting DR5 agonists against homogeneously sensitive tumor cells (having cytotoxicity above apoptotic threshold) will generate superior anti-tumor cell-death (FIG. 3A). Aftereffect, with every single tumor cells eliminated (such as in DR5 sensitive homogenous cellular xenografts in immunodeficient mice; Camidge, 2008; Kaplan-Lefko et al., 2010; Motoki et al., 2005; Zhang et al., 2007)), surface PD-L1 will potentially have limited consequence even if PD-1 expressing T-cells were present. Considering that strong PD-L1 stabilization was evident in UCD52 mixed TNBC PDX tumors (FIGS. 1K and 1L) and that all human tumors are heterogeneous and given that all tested DR5 agonists have failed in clinical trials, a scenario can be envisioned where surface stabilized PD-L1 on dying (highly DR5 sensitive) tumor cells influences the function of neighboring non dying cells (not DR5 sensitive) having cytotoxicity below apoptotic threshold (FIG. 3A). In this scenario, stabilized PD-L1 has potential to suppress the activity of incoming cytotoxic T-cells in tumor microenvironment (TME) and it will give survival advantage to DR5 resistant tumor cells (FIG. 3A).

To investigate above, we tested the hypothesis if surface PD-L1 from dying cells is shuttled to resistant and non-dying cells via apoptotic cell-derived extracellular vesicles called ApoEVs (Caruso & Poon, 2018; Gregory & Dransfield, 2018). We confirmed significantly high PD-L1 presence in ApoEVs after DR5 agonist treatment using dot blot and western immunoblotting (FIGS. 3B-3D).

Next, we made use of L-homopropargylglycine (HPG, a methionine analogue) incorporation instead of methionine in cultured cells (Calve et al., 2016). By making use of HPG specific catalyzing dye in flow cytometry studies, we confirmed PD-L1 transfer from WT cells to DR5-KO cells via ApoEVs (FIGS. 3E and 3F). To this end, we isolated ApoEVs from DR5-WT cells (grown in HPG+/Met− or Met+ media) after agonist antibody treatments. Next we incubated Met+ and HPG+ ApoEVs with DR5-KO cells for 48 hrs. PD-L1 signal with HPG selective dye in R5-KO cells (grown in absence of HPG) confirmed PD-L1 transfer from WT cell (grown in HPG+ culture media; FIG. 2F). Next we tested ApoEVs mediated PD-L1 transfer kinetics using a time course experiment. We observed a significantly high surface (FIG. 3G) and total PD-L1 levels (FIG. 3H) after 24-48 hrs incubation times. As expected, direct DR5 antibody treatments did not stabilize PD-L1 on DR5-KO cells (FIG. 3I). Furthermore, when tested next to each other, unlike DR5 agonist antibodies, TNF-α signaling which also stabilized PD-L1, did not shuttle significant amount of PD-L1 via ApoEVs (FIG. 12). Importantly, DR5-KO cells incubated with ApoEVs showed significantly reduced luciferase activity when co-cultured with PD-1+ jurkat cells (FIG. 3J) confirming PD-1 mediated T-cell inhibition by ApoEV transferred PD-L1. Collectively, these results strongly support orchestration of DR5 agonist mediated PD-L1 stabilization in heterogenous culture conditions due to ApoEVs mediated DR5 transfer from extrinsic apoptotic sensitive cells to resistant cells.

As many clinically failed DR5 agonist activate apoptosis below the tumor clearance threshold (Ashkenazi, 2015), next we asked if PD-L1 stability is maintained after DISC activation in absence of complete execution of cell death. To this end we established condition by making use of Z-DEVD, which preferentially inhibits mainly autocatalytic function of caspase-3 required for its conversion from 20 kiloDalton (kDa) form into fully activated 17 kDa form (Ponder & Boise, 2019). With KMTR2 and Z-DEVD co-treatments, we observed steady activity of caspase-8 (as evident by its cleavage), while the activity of prodomain containing cleaved caspase-3 was inhibited, if Z-DEVD was added to the cultures within 40 minutes of DR5 agonist (KMTR2) treatment. The latter is evident by loss of caspase-3 autocatalytic event (lack of accumulation of 17KDa; see lane 4-6 of FIG. 3K), loss of PARP cleavage (see lanes 4-6 of FIG. 3K). The results were also supported by caspase-3 activity assays (FIG. 3L) and cell death assays (FIG. 3M). Importantly S5a levels were also reduced and PD-L1 was stabilized in lysates despite lack of effective cell death (FIG. 3K, lanes 2-6). These findings are also suggestive that DR5 agonist antibodies with high extrinsic ability to generate activated DISC-caspase-8 (despite not executing cell-death above tumor clearance threshold) can also potentially stabilize PD-L1. The latter could be a contributing factor in tumor cells having dysregulation of downstream prosurvival proteins such as Bcl-2 (and Bcl-xL etc.) despite optimal DR5 activation.

### Example 4

### DR5 Agonist Activated ROCK1 is Required for PD-L1 Surface Mobilization

A Phase II combination trial of paclitaxel and DR5 agonist tigatuzumab in TNBC patients has described upregulation of genes involved in apoptotic blebbing (Forero-Torres et al., 2015). Strikingly, activation (cleavage) of ROCK1, the key regulator of membrane blebbing, cytoskeletal ruffling and vesicular exocytosis (Coleman et al., 2001) was maintained (FIG. 3K) despite autocatalytic blockade of caspase-3, PARP and loss of cell-death (FIGS. 3L and 3M), suggesting potential role of DISC-caspase-8 in activating ROCK1 downstream of DR5 agonists. Interestingly, a very recent study discovered ROCK1 (Rho associated coiled coil containing kinase-1) to be PD-L1 associated regulator using ingenuity pathway analysis (Chan et al., 2019). Furthermore, ROCK1 is PD-L1 coexpressed gene in melanoma patients (Madore et al., 2016).

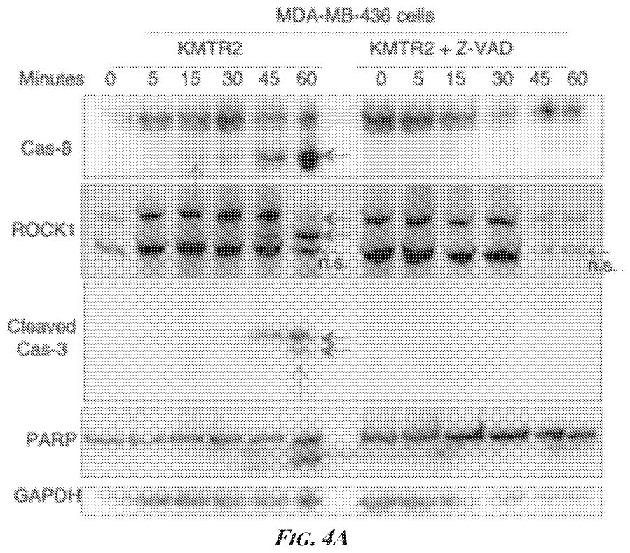

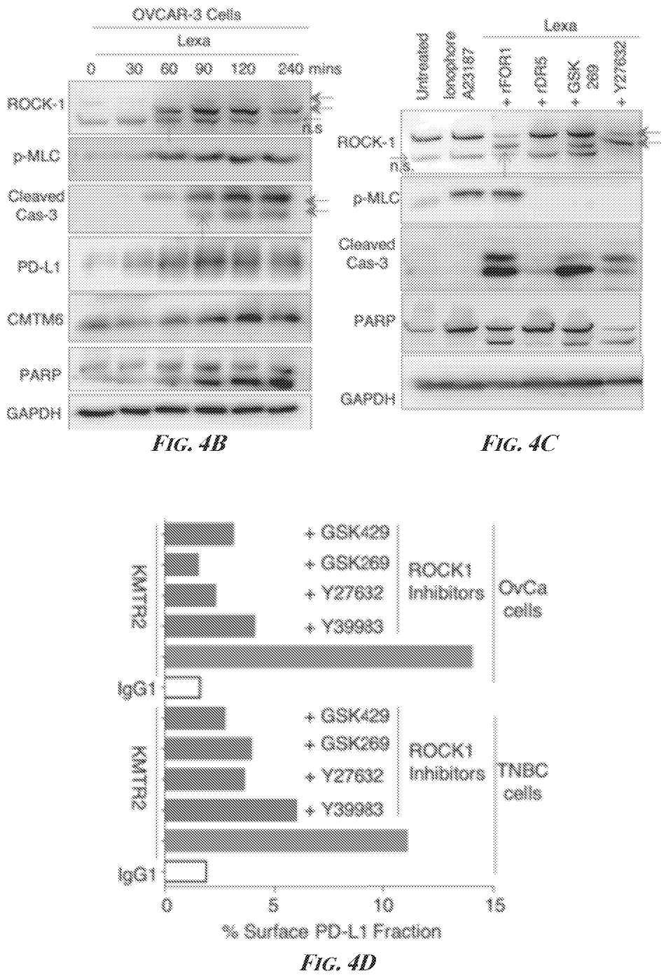

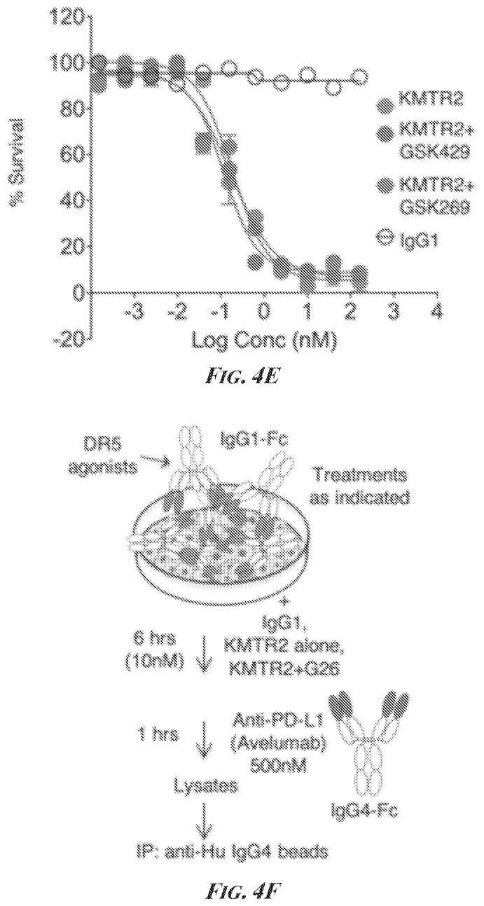

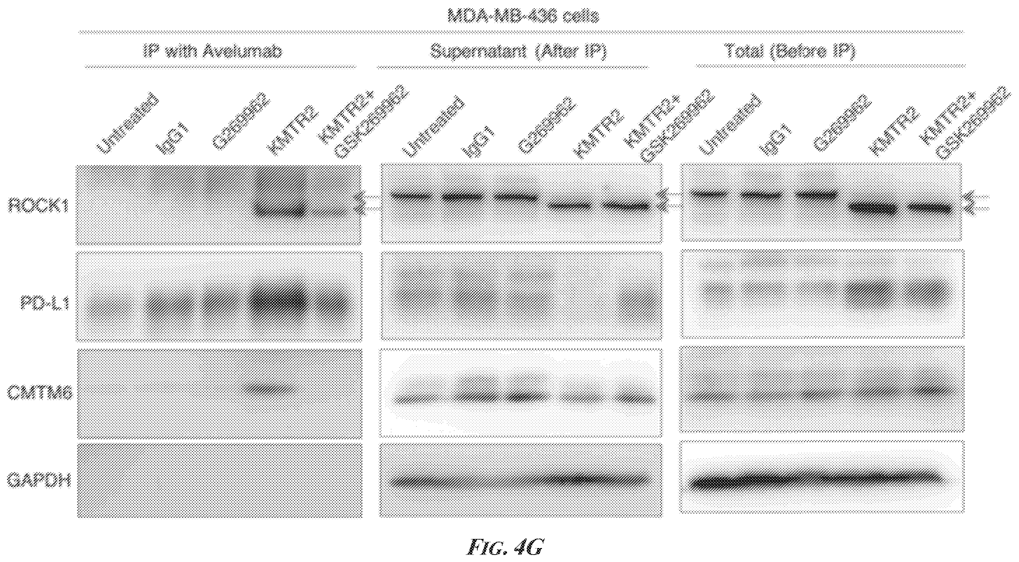

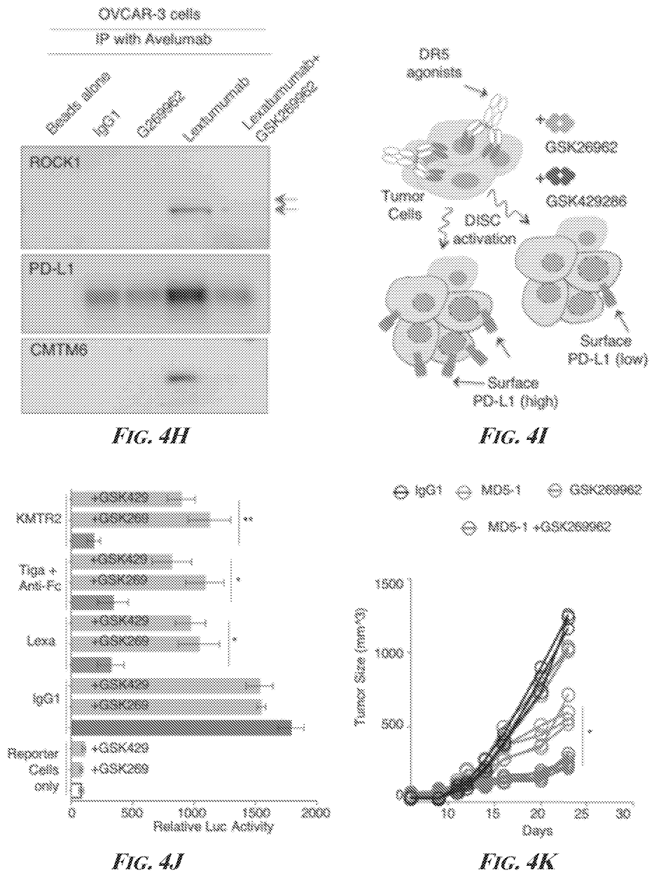

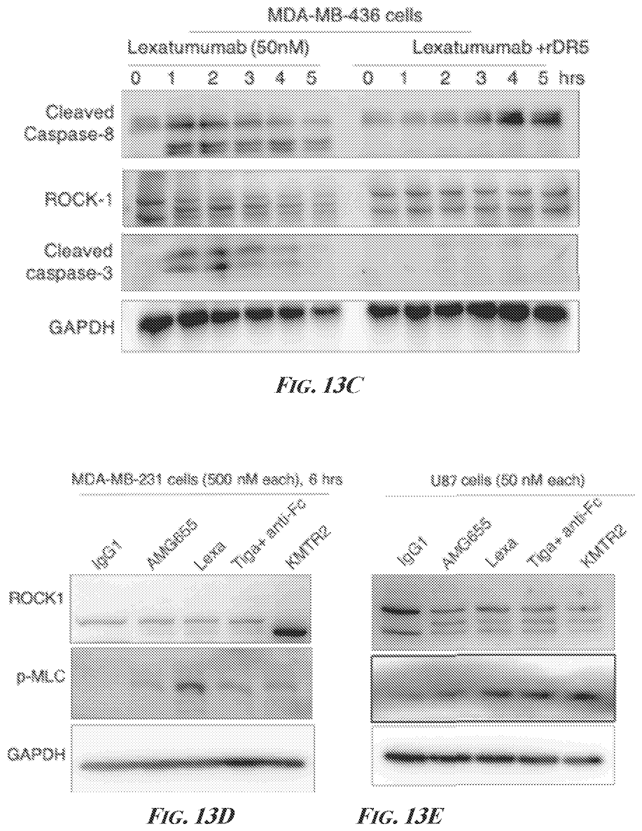

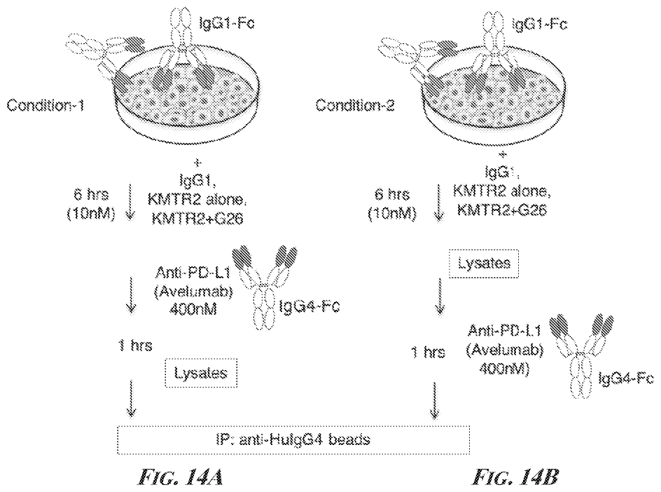

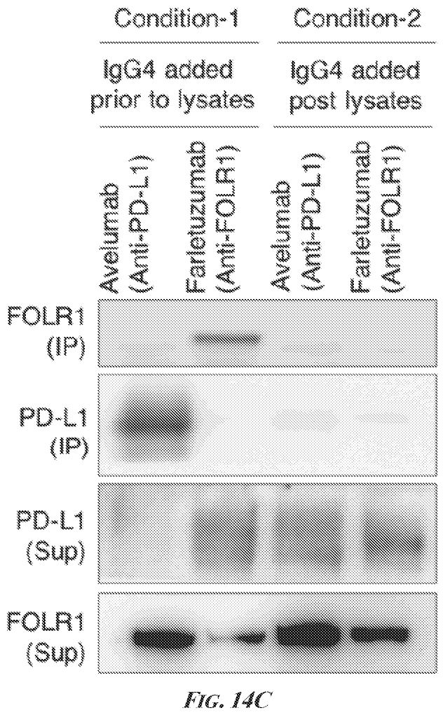

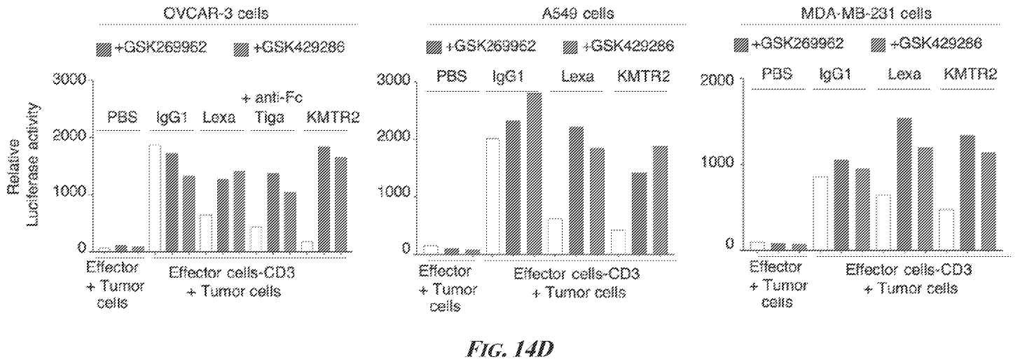

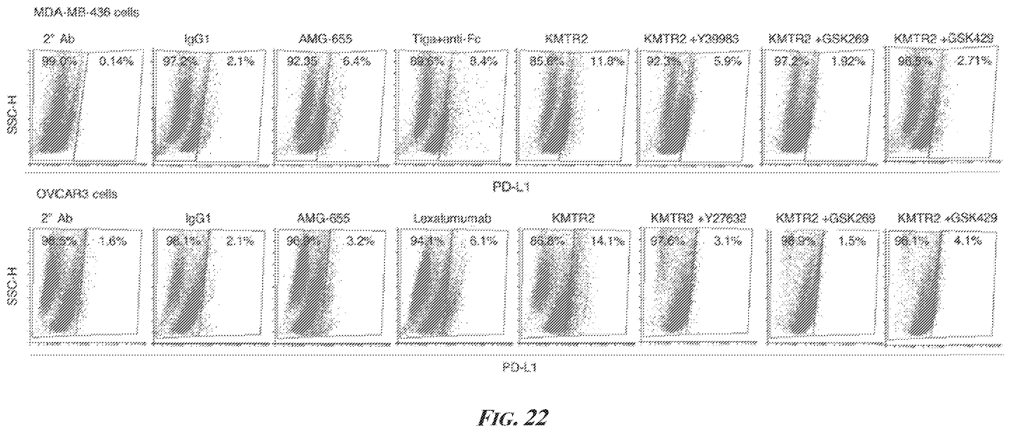

Given that PD-L1 must be kept away from lysosome (Burr et al., 2017) and proteasome (Lim et al., 2016) for its stability, we next investigated if DR5 agonists require ROCK1 function to surface mobilize PD-L1. We discovered early activation of ROCK1 and myosin light chain phosphorylation by all DR5 agonists in various tumor lines (FIGS. 4A-4C and 13A-13E). Similar to our observations with Z-DEVD inhibitor (FIG. 3K), a partial ROCK1 cleavage was evident prior to full caspase-3 activation (FIGS. 4A, 4B, 13A, and 13B, compare vertical arrows in lanes). These results indicated that in scenarios where caspase-3 function is inhibited, the upstream DISC-caspase-8 also have potential to activate ROCK1 pathway. Regardless, various ROCK1 inhibitors (ROCK1i) downregulated surface PD-L1 levels without effecting cell-death (FIGS. 4D, 4E, and 22). In addition, during native immunoprecipitation (IP) studies with clinical anti-PD-L1 antibody (avelumab), activated ROCK1 directly interacted with surface PD-L1 in a complex that also contained CMTM6 (FIGS. 4F-4H and 14A-14C). Interestingly, DR5 agonist+ROCK1 inhibition did not affect the stabilized PD-L1 levels in cellular lysates (FIG. 4G, lane 5th lane, right blot), confirming PD-L1 intracellular stabilization being independent of ROCK1 activity but dependent on DISC-caspase-8 function. When DR5 treated tumor cells and jurkat co-culture reporter assays were evaluated in presence of ROCK1 inhibitors, luciferase activity was significantly enhanced in lieu with loss of surface PD-L1 (FIGS. 41, 4J, and 14D).

Next, we tested ROCK1 inhibitor GSK269962 (2 mg/kg) in combination of murine DR5 agonist MD5-1 (50 μg dose) in 4T1 syngeneic TNBC tumor models. We observed significant tumor reduction by antibody drug combination over MD5-1, while GSK269962 alone was ineffective (FIG. 4K). Taken together, these findings demonstrated that ROCK1 downstream of DR5 agonists played an important role to help mobilize (potentially via CMTM6 or other unknown mechanisms) the internally stabilized PD-L1 on tumor cell-surface.

### Example 5

### Generation of Chimeric Receptor to Test Clinical Human DR5 Agonists in Immunocompetent Mouse Models

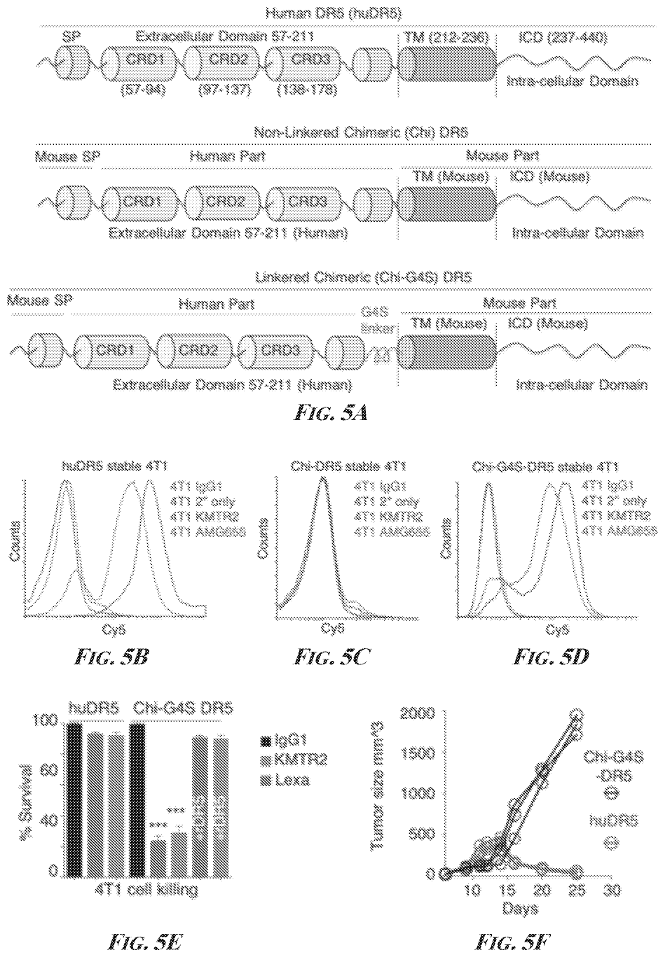

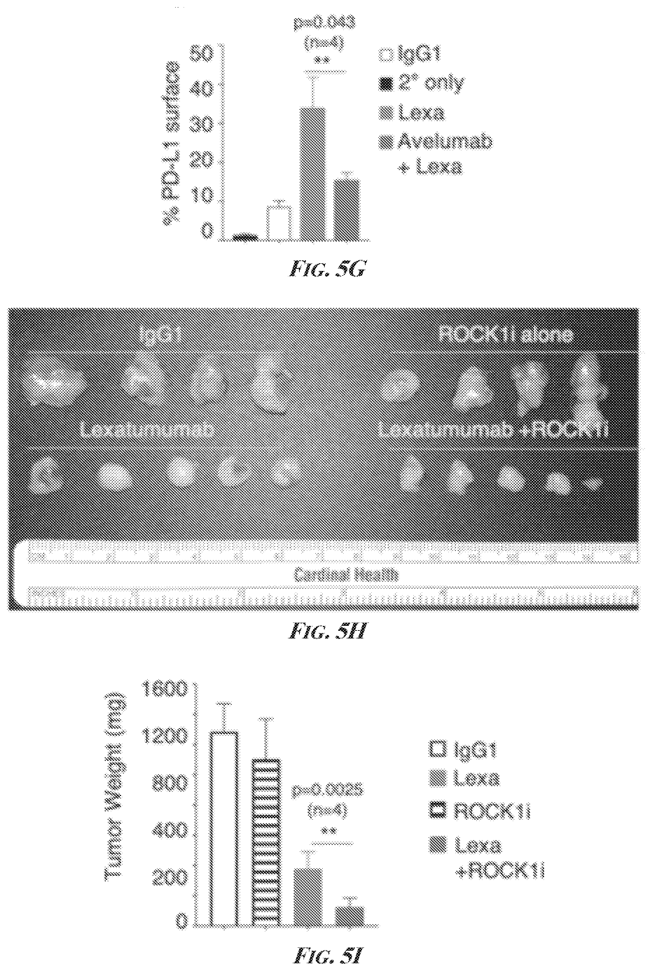

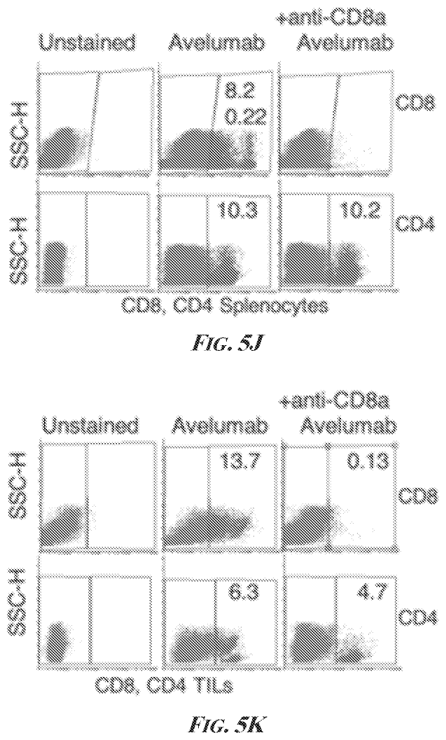

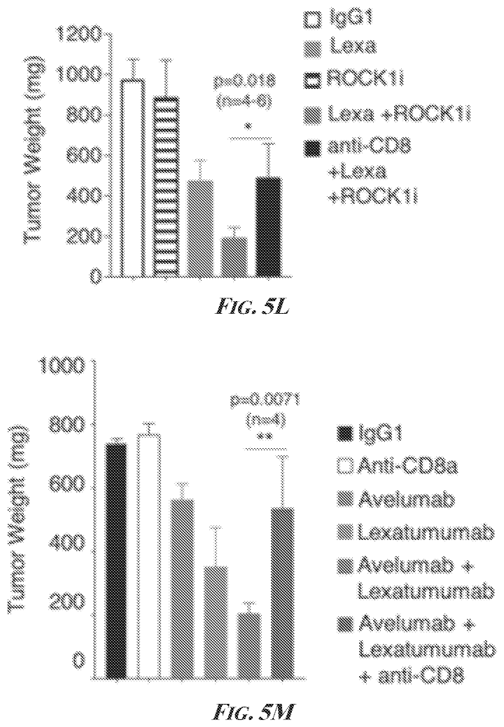

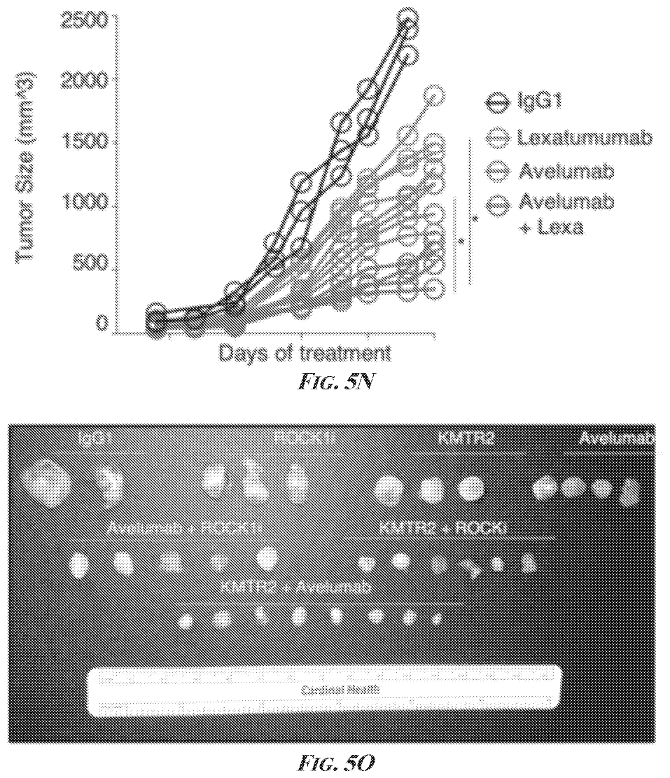

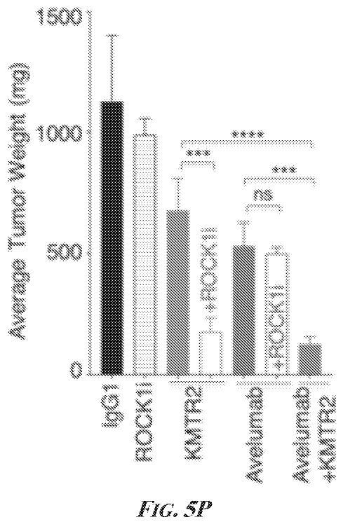

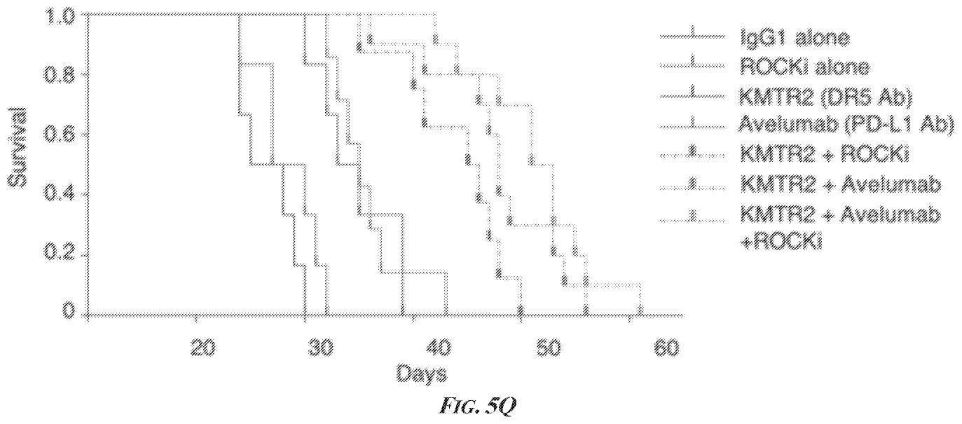

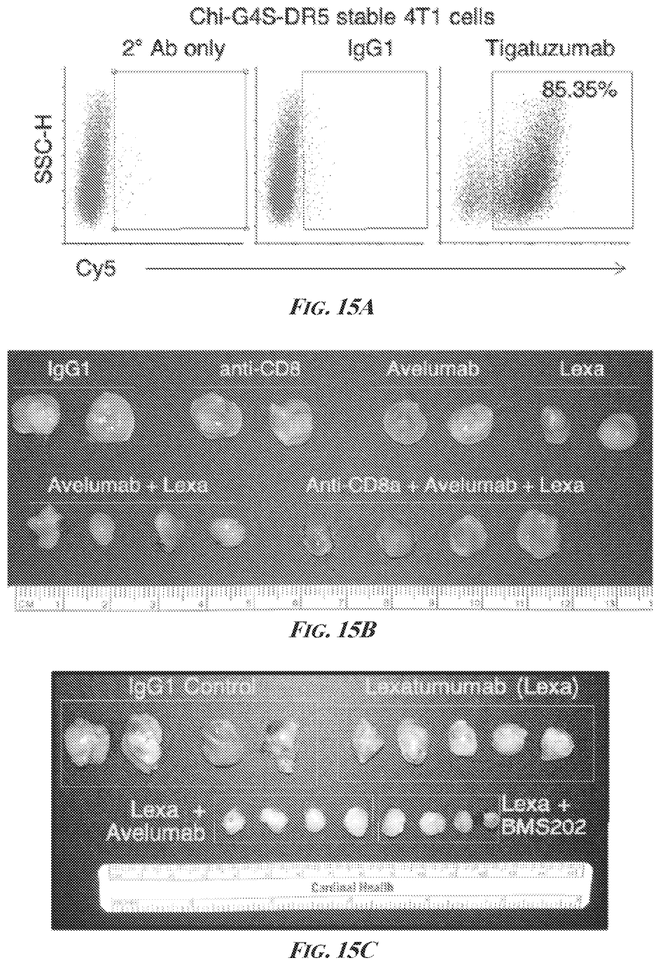

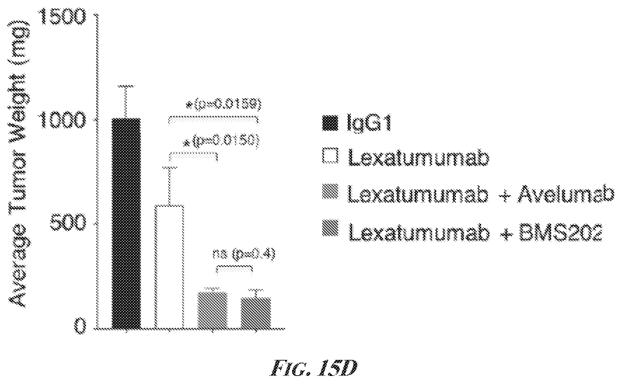

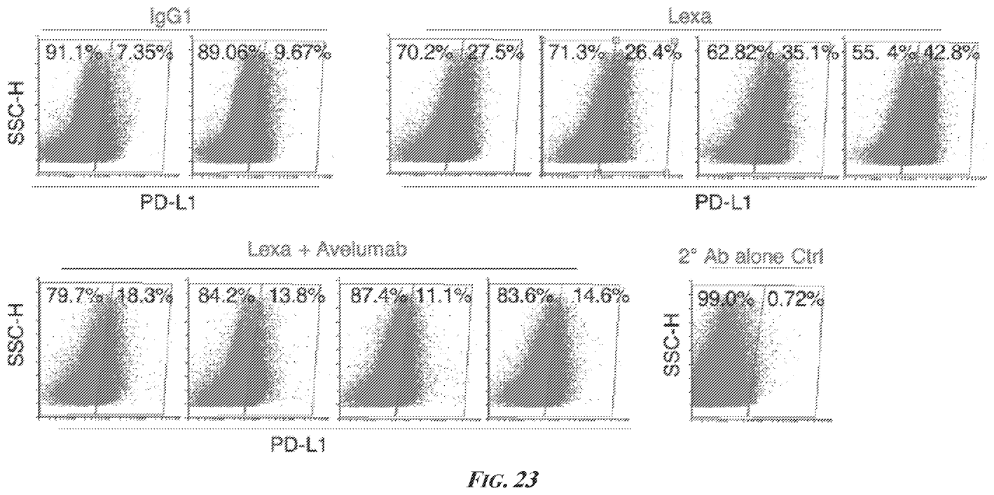

To investigate clinical human DR5 agonist using in vivo heterogeneous mixed tumor graft condition, we engineered chimeric DR5 constructs, with extracellular domain (ECD) of human DR5 fused with mouse DR5 transmembrane (TM) and intracellular domain (ICD; FIG. 5A). These constructs were expressed in murine 4T1, MC38 and ID8 cells using lentiviral infection. We observed mixed stable lines with >85% cells being positive for DR5 (FIG. 15A). Interestingly, full-length human DR5 (huDR5) and G4S linker fused (chi-G4SDR5) expressed in murine cells, while a direct fusion (chi-DR5) was not stable (FIGS. 5B-5D). In terms of activity, consistent with its expression, only chi-G4S-DR5 was activated with clinical DR5 agonist antibodies to induce ˜75-85% cell death (FIG. 5E). When grafted on Balb/c animals only 4T1 chi-G4S-DR5 cells formed tumors while huDR5 stable 4T1 tumors were rejected (FIG. 5F). Lexatumumab treated chi-G4S-DR5 mixed tumors had higher overall PD-L1 on cell surface, while co-treatment with clinical anti-PD-L1 antibody avelumab (human/mouse cross-reactive) inhibited surface presence of PD-L1 (FIGS. 5G and 23). This is in agreement with published reports of glycosylated PD-L1 internalization after binding to activity blocking anti-PD-L1 antibodies (Lee et al., 2019).

### Example 6

### DR-5-ROCK1/DR5-PD-L1 Co-targeting Improves Efficacy of Clinical Human DR5 Agonists

Consistent with higher PD-L1 in chi-G4S-DR5 stable 4T1 tumors (FIG. 5G), DR5 agonist and ROCK1 inhibitor co-treatment (similar to MD5-1-ROCK1 co-targeting), showed higher anti-tumor efficacy (FIGS. 5H and 5I, tumors isolated after 7 doses) as compared to lexatumumab alone. To confirm that surface enhanced PD-L1 in chi-G4S-DR5 stable 4T1 tumor works by inhibiting effector CD8+ T-cell function via PD-1 engagement, next we depleted CD8+ T cells in tumor bearing animals (FIGS. 5J and 5K). In vivo depletion of CD8+ T cells abrogated the combinatorial efficacy of lexatumumab and ROCK1 inhibitor (FIG. 5L, tumors harvested after 6 doses). Similar higher anti-tumor efficacy results were obtained when lexatumumab (or KMTR2) were used either in combination of anti-PD-L1 avelumab antibody (FIGS. 5M-5P) or a PD-1 inhibitor (FIGS. 15C and 15D). As expected, depletion of CD8 cells in tumor bearing animals also abrogated combinatorial lexatumumab and avelumab efficacy (FIGS. 5M and 15B, tumors harvested after 6 doses). We also carried out animal survival studies with double and triple co-targeting of DR5-PD-L1-ROCK1 co-targeting. Consistently double and triple combinations significantly improved survival as compared to DR5 agonist alone or anti-PD-L1 alone (FIG. 5Q).

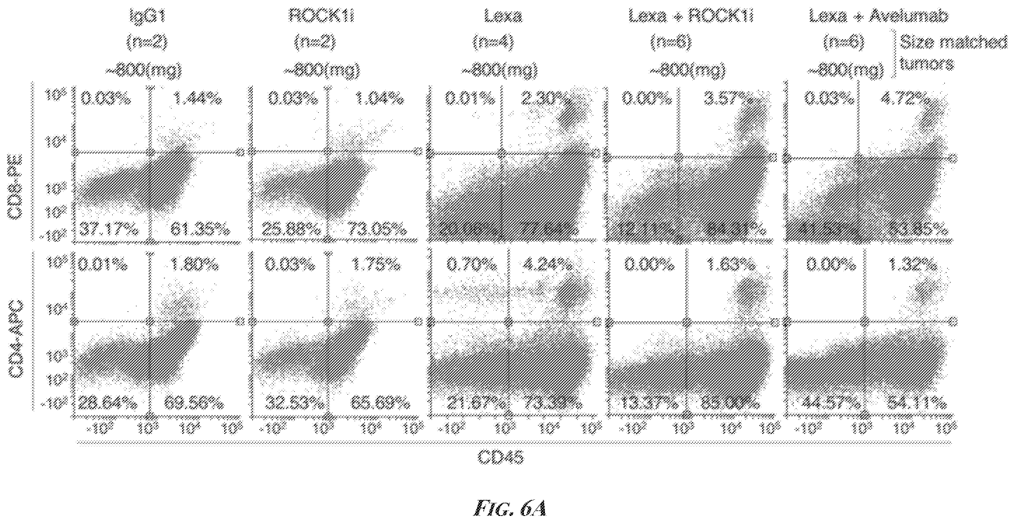

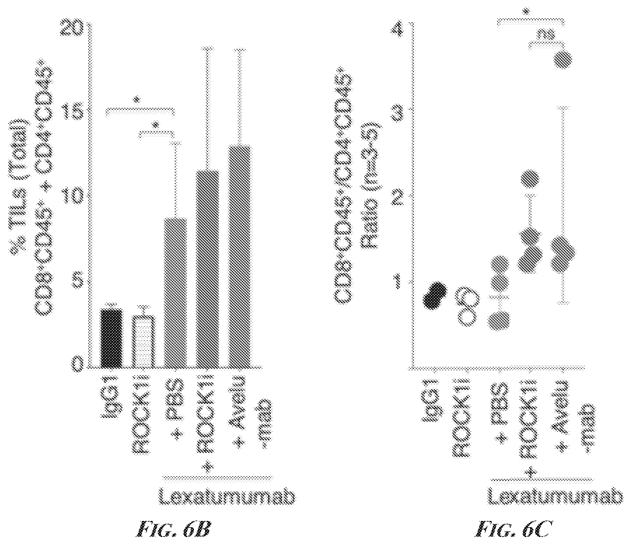

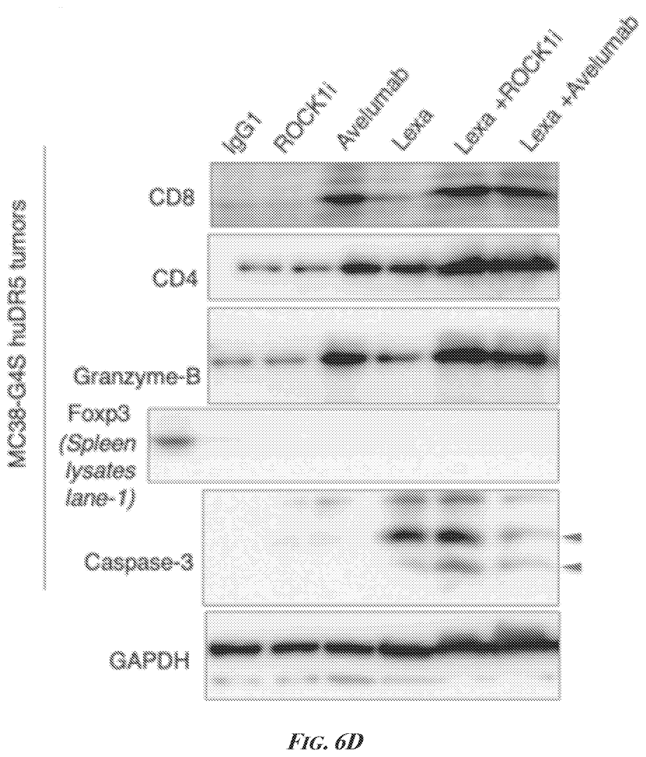

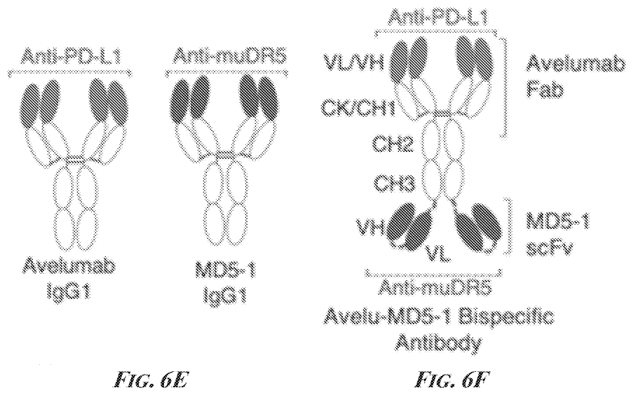

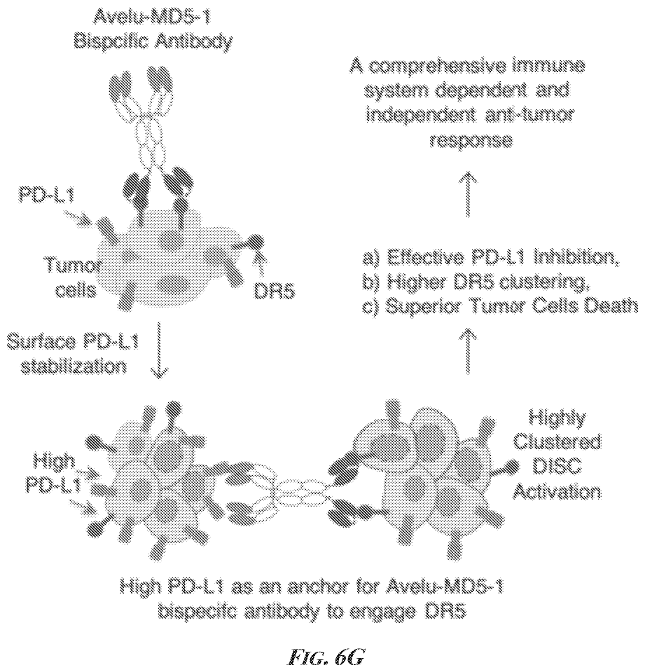

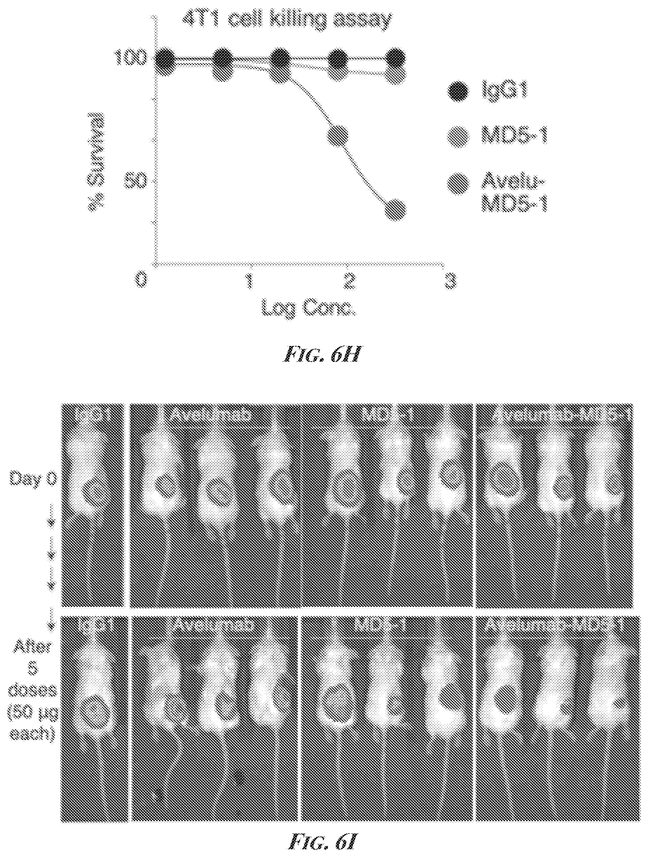

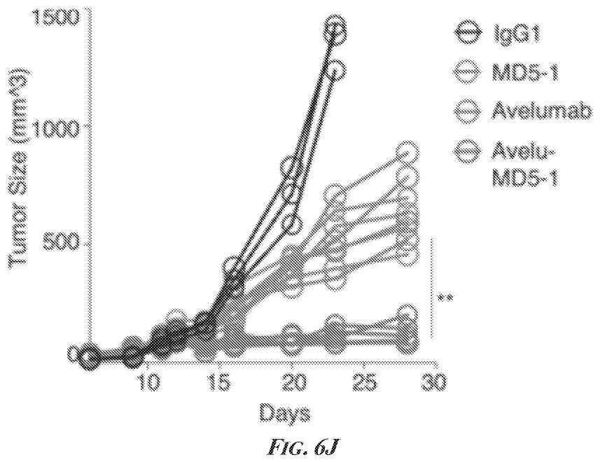

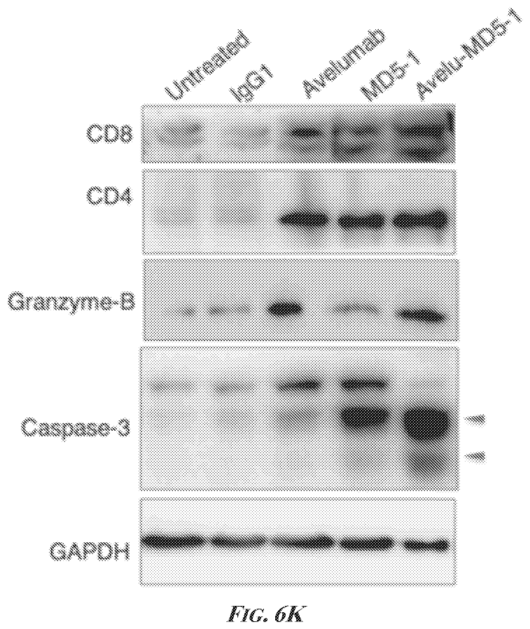

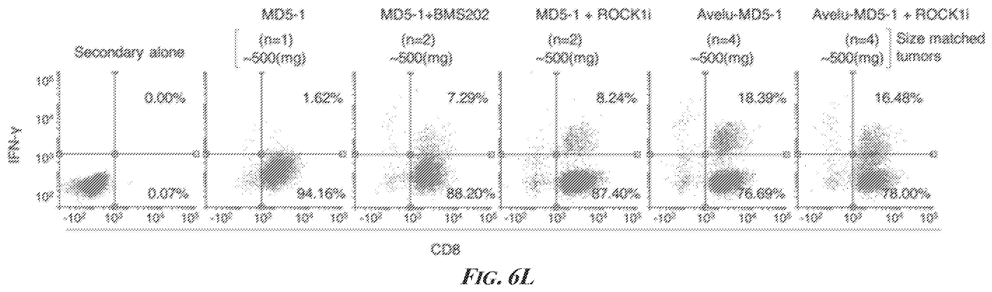

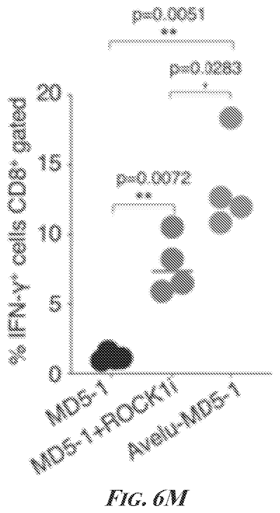

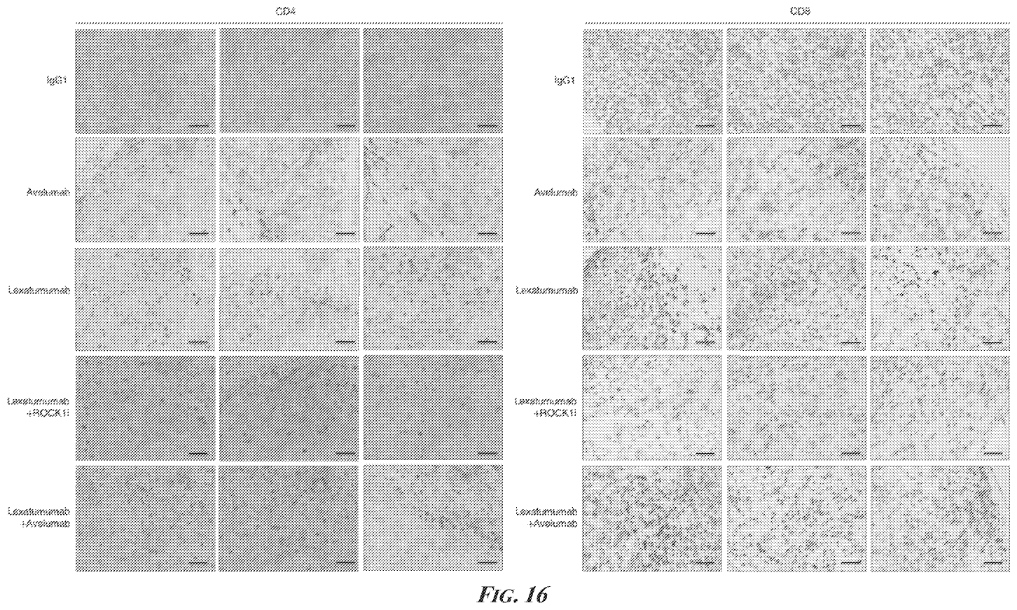

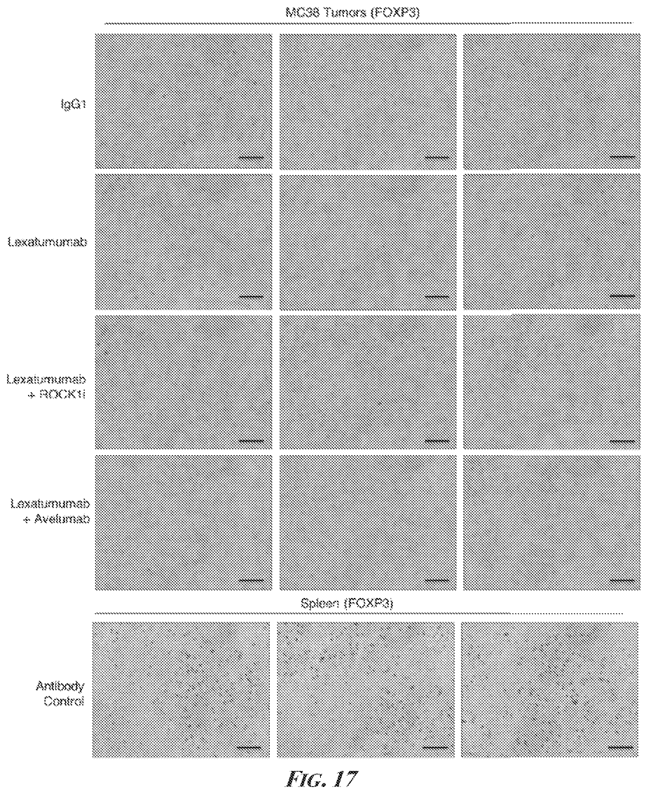

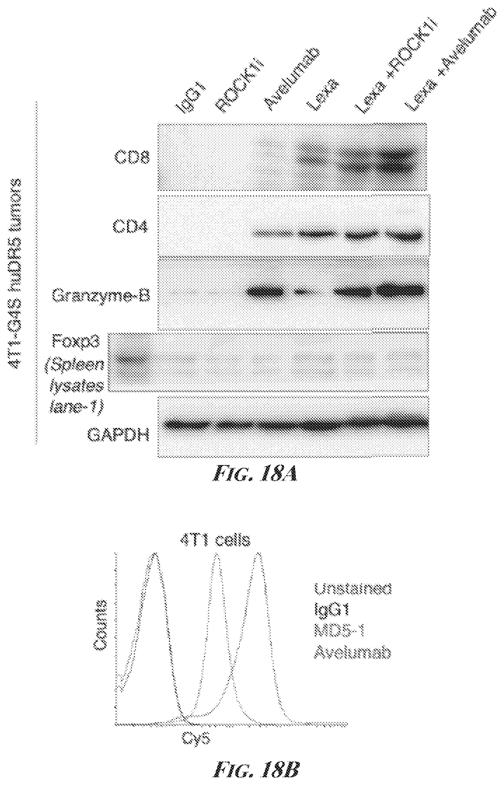

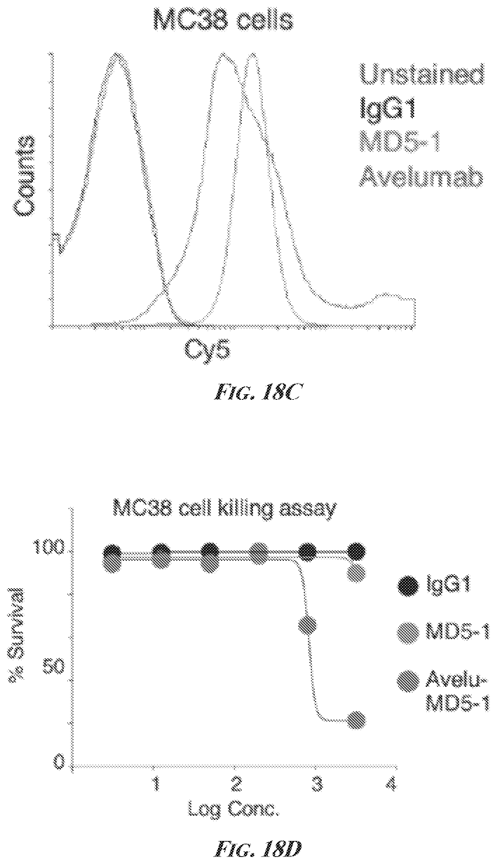

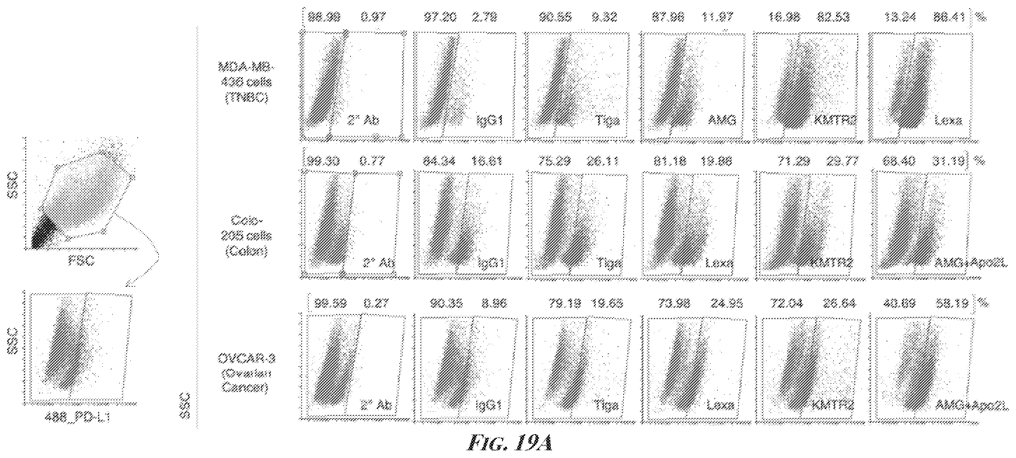

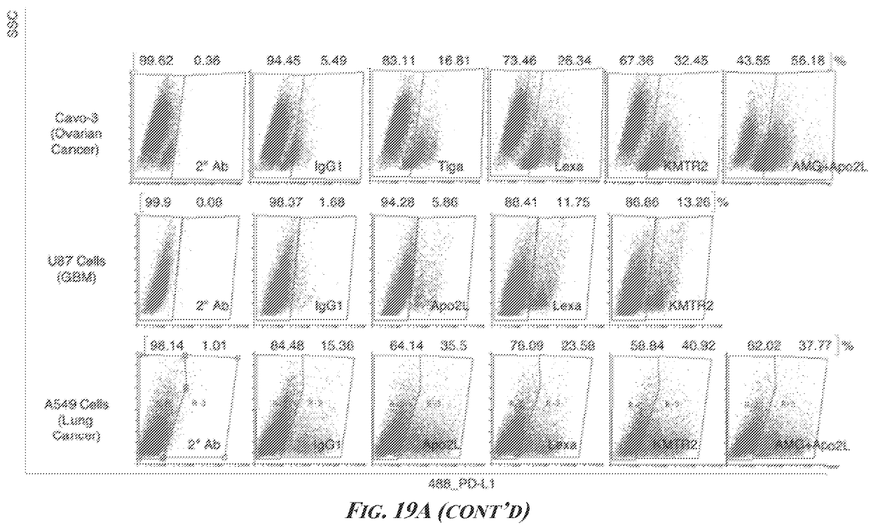

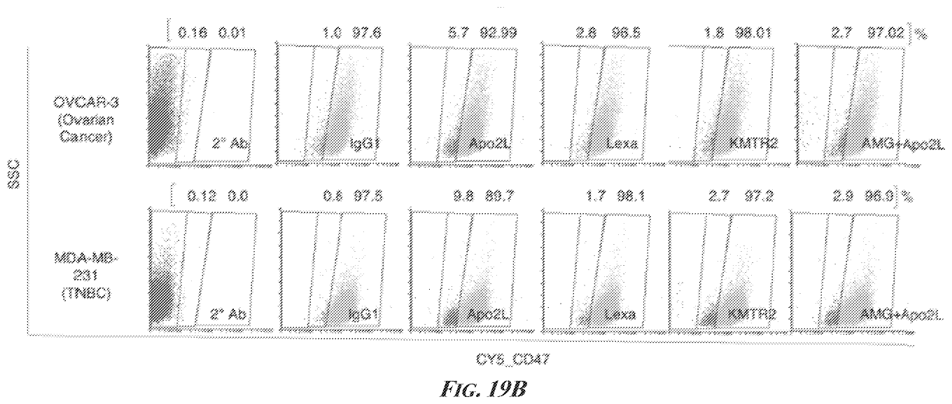

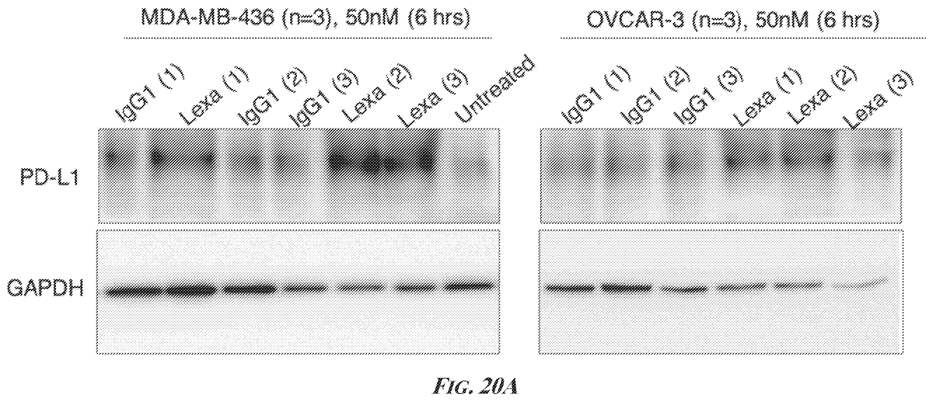

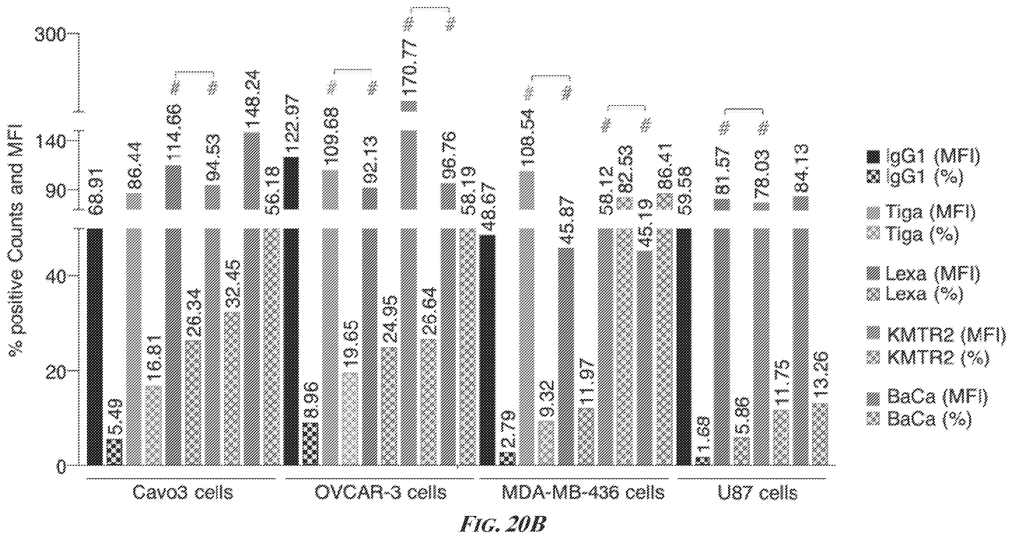

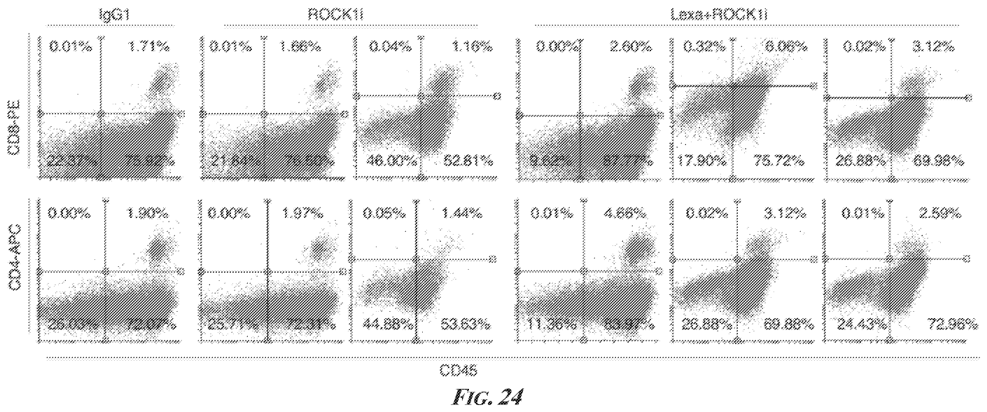

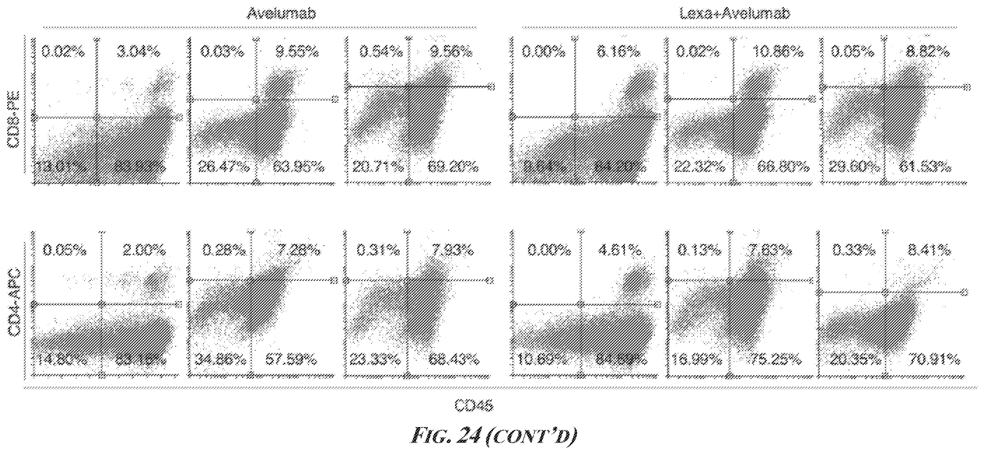

To further confirm the direct role of intratumor effector CD8+ T-cell in improved combinatorial efficacy, animals were treated with 4 doses of DR5 agonist+ROCK1 inhibitor (and avelumab). Next, size matched tumors were later subjected to tumor infiltrating leukocyte (TIL) enrichment using Ficoll-Paque method as described previously (Tan & Lei, 2019) followed by dual staining T-cells analysis using flow cytometry. We observed significant enriched of CD8+CD45+ and CD4+CD45+ T-cells in DR5 agonist treated tumors regardless of ROCK1i and avelumab co-treatments (FIGS. 6A, 6B, and 24; n=6-20 tumor bearing animals). Similar results of immune infiltration were also evident in CD8, CD4 IHC studies of chi-G4S-DR5 stable MC38 tumors treated with either DR5 agonist alone or in combination of ROCK1i or avelumab (FIG. 16). These observation supports tumor breakdown (debulking) function of DR5 agonists to enhance immune infiltration. Based on numerous clinical trials, a higher ratio of cytotoxic T-cells over helper T-cells has been described the key prognostic predictor of effective anti-tumor response in TNBC and other tumors (Wang et al., 2017a). Strikingly, we observed a higher CD8+/CD4+ ratio in lexatumumab +ROCK1i and lexatumumab+avelumab treated 4T1 tumors but not in DR5 agonist alone treated tumors (FIGS. 6C and 24; n=6-20 tumor bearing animals). Similar results of significantly higher granzyme-b activity (a marker of cytotoxic T-cell function) were evident in avelumab, lexatumumab +ROCK1i and lexatumumab+avelumab treated size matched tumors as compared to DR5 agonist alone treated chi-G4S-DR5 stable MC38 and 4T1 tumors (FIGS. 6D and 18A). Interestingly, DR5 agonist co-treatments did not enhance activity of regulatory T-cells (T-regs) in tumors as evident with Foxp3 western blots and IHC data (FIGS. 6D, 18A, and 17). Collectively, these sets of comprehensive investigations collective support higher tumor breakdown and higher anti-tumor immune function of DR5 agonists when given in combination of ROCK1 inhibitors or PD-L1 function inhibiting antibodies.

### Example 7

### DR-5-PD-L1 Co-targeting Bispecific Antibody for Solid Tumors

PD-L1 is a key regulator of immune-suppression in TNBC and other solid cancers, and FDA has approved various PD-L1/PD-1 blocking antibodies (Aktas et al., 2019). As DR5 agonist stabilized PD-L1 on cell surface, we next hypothesize that elevated surface PDL1 if used as an anchor, will not only activate T-cells but will also use PD-L1 as an anchor to enhanced DR5 signaling (Shivange et al., 2018). Thus, we next genetically engineered avelumab and murine DR5 agonist MD5-1 antibody into bispecific antibody called avelu-MD5-1 (FIGS. 6E and 6F), capable of not only blocking PD-L1 immunosuppressive function but also enhancing DR5-DISC clustering (FIG. 6G). Both antibodies were confirmed to have binding against murine MC38 cells and 4T1 cells (FIGS. 18B and 18C). MD5-1 antibody requires Fc crosslinking to activate cell death (Shivange et al., 2018) while avelu-MD5-1 was effective in killing murine 4T1 and MC38 cells (FIGS. 6H and 18D). When tested in 4T1 TNBC syngeneic tumor models, avelu-MD5-1 completely regressed tumors (FIGS. 6I and 6J). Both MD5-1 alone and avelumab alone only stabilized the tumors at similar dose (FIGS. 6I and 6J).

Next, size matched MD5-1, avelumab and avelu-MD5-1 tumor lysates (after 6 doses) were analyzed for granzyme-b and caspase-3 activity (FIG. 6K). Similar to previous results of lexatumumab (FIG. 6D), MD5-1 treated tumor lysates had high CD4 and CD8 expression as compared to IgG1 treated tumors. However, the granzyme expression in MD5-1 lysates was similar to IgG1 treated tumors, while both MD5-1 and avelu-MD5-1 lysates had elevated cleaved caspase-3 levels (FIG. 6K).

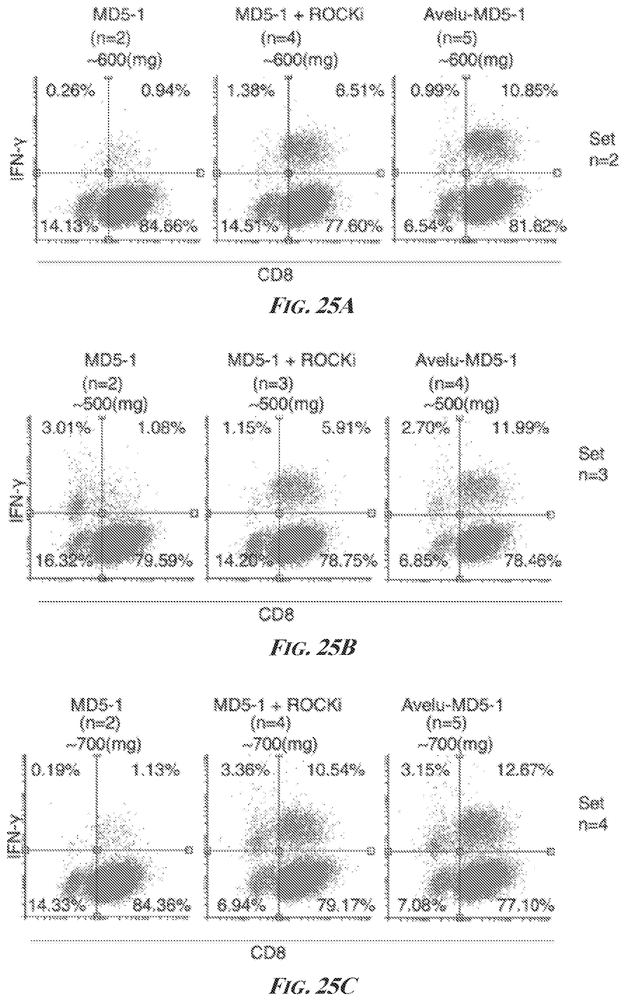

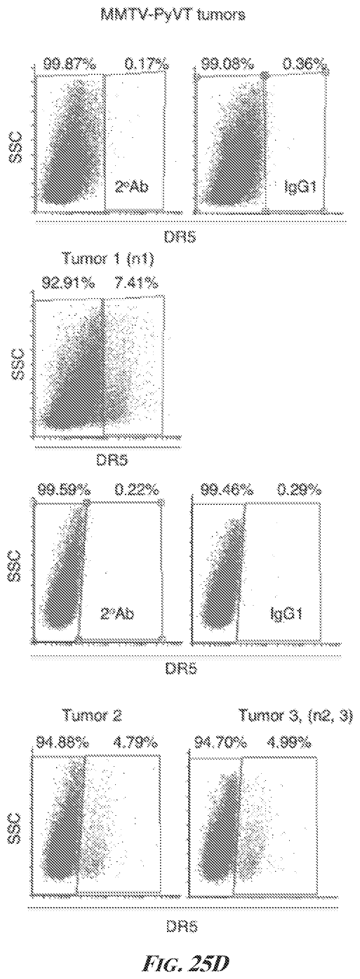

Next, we analyzed for intracellular IFN-γ expression (an indicator of cytotoxic T-cell activity) in enriched CD8 population using flow cytometry studies. We observed a significantly higher percentage (multiple-fold) of IFN-γ positive CD8 cells in MD5-1+ROCK1i, MD5-1+PD-1 inhibitor (BMS202) and avelu-MD5-1 bispecific treated tumors as compared to DR5 agonist (MD5-1) alone (FIGS. 6L, 6M, and 25A-25C; n=7-16 tumors). Collectively, these results raised a high expectation of potentially significantly effective clinical response in TNBC patients by FDA approved anti-PD-L1 atezolizumab if given in combination of DR5 agonists (Aktas et al., 2019).

### Discussion of Examples

Over last few decades, the faithful translation of many preclinical studies into human clinical trials have largely relied on homogenous cellular and heterogeneous patients derived tumor xenograft models. In current era of cancer immunotherapy, these tumor models present significant hurdles to fully understand the complex cancer immunotherapy strategies by antibodies that eliminate tumor cells by apoptotic cytotoxicity. As immune activating or repressive function of human DR5 agonist antibodies remains largely untested and since all DR5 agonist have failed to move beyond phase-II trials (Ashkenazi, 2015), we undertook these investigations to underpin the potential immune inhibitory function of DR5 agonists if any.

The presently disclosed findings with various solid tumor cell lines, multiple solid tumor xenografts, and using multiple clinical DR5 agonist antibodies support PD-L1 stabilization and immunosuppressive role of DR5 agonists. PD-L1 surface mobilization was also maintained regardless of EMT acquisition in tumor cells. Using an array of experimental, preclinical and clinical DR5 agonists antibodies targeting mouse, human and chimeric DR5, our findings have discovered an unexpected PD-L1 based immune evasion mechanism that is not selective to a particular DR5 antibody, which binds to a particular DR5 epitope. Rather, the immune evasion is driven by DISC-caspase-8 signaling. Consistent with previous reports, our results support caspase mediated cleavage of proteasome complex (Cohen, 2005). Moreover, in agreement with previous reports of PDL1 regulation by deubiquitinase CSN5 (Lim et al., 2016), proteasome inhibition and deubiquitinase activity serve the same purpose via different upstream mechanisms.

As lexatumumab, apomab, AMG655, and tigatuzumab have been tested clinically in gastric cancers, TNBC, and other solid cancers (Kalthoff & Trauzold, 2009), future IHC and ISH studies from treated patient samples will strengthen the results in human. Unfortunately, we could not get access to DR5 agonist treated patient samples. Regardless, in grafted TNBC PDX tumors, we observed high PD-L1 in IHC studies after DR5 agonist treatments and their co-targeting (PD-L1+DR5) significantly improved immune effector function and anti-tumor efficacy. Continuing on the similar note, an appropriate immune competent tumor model remains a challenge in DR5 field. Indeed, when tested using MMTV-PyVT TNBC GEM tumor model (Usary et al., 2016), less than 10% cells stained positive for DR5 as compared to surrogate 4T1, MC38 syngeneic models (FIG. 25D). Furthermore, these breast TNBC GEM models (MMTV-PyVT, MMTV-neu) express undetectably low PD-L1 (<5%) on tumor cells (Nolan et al., 2017) and do not respond to PD-1/PD-L1 and anti-CTLA4 immune checkpoint therapies unless given in combination of adjuvants such as cisplatin (Nolan et al., 2017). Thus, described transgenic syngeneic tumor studies in this paper is very first report in the field to test human clinical DR5 agonists in immune competent tumor microenvironment.

Our findings of S5a proteasome 26s subunit degradation and ROCK1 activation by activated caspase-8 without amplification of caspase-3 and downstream cell-death are intriguing as the cellular resistance due to Bcl-2 (Bcl-x) and other pro-survival proteins remains a critical clinical challenge to success of DR5 therapy (LeBlanc et al., 2002). In addition, various reports have described non-apoptotic function of caspase-8 during development(Miura, 2012; Solier et al., 2017), particularly its key role in T-cell development and immune homeostasis by another TNF superfamily Fas ligand signaling pathway (Chun et al., 2002; Salmena et al., 2003). Importantly, random caspase cascade activation by any other apoptotic agent that act through mitochondria are not involved in leukocyte development (Chun et al., 2002; Salmena et al., 2003). Furthermore, as caspase-8 is activated by both pro-survival and pro-death signals (Newton et al., 2019), the function of partially activated caspase-8 (by antibodies that activate apoptosis below tumor clearance threshold) in regulating proteasome activity and cytoskeleton without activating cell-death, indicate its differential regulation in cancer cells than immune cells (Ferrari et al., 1998). If clinical proteasome inhibitors induce immune suppression in patients is beyond the scope of described investigations.

Between rho binding domain (RBD) domain and pleckstrin homology (PH) domain, although ROCK1 contains two optimal caspase-8/caspase-3 cleavage XEXD sites, recent studies have established that either distal regions of substrate itself or other regulators in complex could optimally prefer caspase-8 over caspase-3 (Baker & Masters, 2018). Similarly caspase-8 regulation by two different forms of cFLIP (large and small) has been described, which can result in entirely different outcome (Hughes et al., 2016). As Rho family of GTPases are key player in lymphocyte development and activation, and cancer cell exploiting one of their family member (ROCK1) to escape immune activity is similar to hijacking of macrophage-produced complement C1q to promote their own tumor growth (Roumenina et al., 2019).

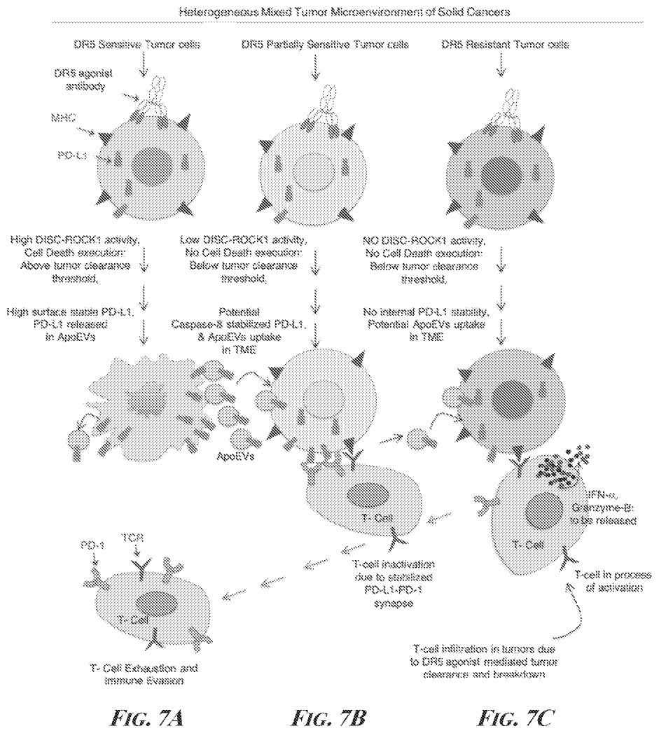

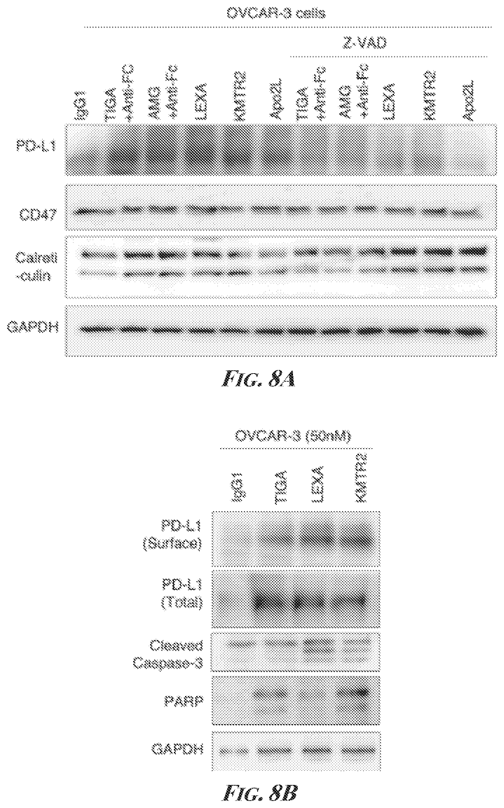

Tumor cells having lower apoptotic threshold not only mobilized PD-L1 on cell surface (both in vitro and in vivo) but also shuttles it to neighboring tumor cells in a process that requires ROCK1 activation (FIGS. 7A-7C). ROCK-1 inhibitors that block metastasis and invasion are already in clinical trials (Chin et al., 2015). How ROCK1 help mobilize PD-L1 to cell surface demands further investigations. If activated ROCK1 makes use of same set of regulators that are important for its established membrane blebbing and cytoskeletal ruffling function (Coleman et al., 2001) or some other mechanisms that potentially also include other regulators such as CMTM6 (FIGS. 4A-4K) to help mobilize PD-L1 to tumor cell surface is beyond the scope of these investigations CMTM6 (Burr et al., 2017). Regardless, these findings of PD-L1 shuttling via ApoEVs are also is in agreement with described studies of immunosuppression by exosomal PD-L1 (Chen et al., 2018). Importantly, besides avoiding immunosuppression, an effective DR5 agonist must also drive superior receptor clustering to eliminate both low and high DR5 expressing tumor cell in heterogeneous solid tumors (Ashkenazi, 2015). The observed dual high caspase-3 and IFN-γ activity (FIGS. 6A-6M) of a DR5 and PD-L1 co-engaging bispecific antibody (compare to MD5-1 or avelumab alone) is a key strategy and is in agreement with previous reports of higher order DR5 clustering by an anchored approach (Shivange et al., 2018). Furthermore, we consistently found that DR5 agonist co-targeting either with anti-PD-L1 or ROCK1i gave the higher immune infiltrations (T-cells) in tumors, however animals treated with DR5+PD-L1 co-targeting had higher overall survival and significantly higher IFN-γ activity as compared to DR5+ROCK1i. We believe the observed differences are due to distinct PD-L1 inhibition working mechanisms in the TME. The anti-PD-L1 antibody blocks both the basal and overall PD-L1 function in TME, while ROCK1 inhibition only regulates intra-cellularly stabilized PD-L1 shuttling to cell surface without changing basal surface PD-L1 levels in TME. Moreover, unlike syngeneic or transgenic surrogate tumors, differential contributing role of immune-inhibitory cytokines from T-regs (Shevach, 2004) and MDSCs (Condamine et al., 2014) could account for additional discrepancies in the TME of different tumor types.

After a comprehensive trial in PD-L1+ patient population, atezolizumab (anti-PD-L1 therapy) was recently approved for metastatic TNBC patients expressing PDL1 (Mavratzas et al., 2020), and since a large proportion of TNBC also express elevated DR5 levels (Forero-Torres et al., 2010), if a bispecific PD-L1-DR5 antibody will further improve survival in TNBC patients need to be seen in clinical trials. Given that key factor determining tumor progression within spatiotemporal dynamics, is the infiltration and activity of cytotoxic T cells in the TME (Binnewies et al., 2018) and considering most preclinical DR5 agonist studies have relied on xenograft models lacking T-cells and TME induced immunological changes in tumors (Camidge, 2008; Kaplan-Lefko et al., 2010; Motoki et al., 2005; Zhang et al., 2007), our results support orchestration of an immune suppression by human and murine DR5 agonists.

In summary, decades of research focused on deregulation of both intrinsic and extrinsic cell death have defined various genetic and non-genetic apoptotic variable factors that contribute to acquired resistance in clinical settings (Spencer et al., 2009). We report an additional PDL1 barrier that escalates T-cell dysfunction and immunosuppression as a function of DR5 agonists in solid cancers. We also find that ROCK-1, downstream of DR5 agonists, help regulate surface mobilization of PD-L1 in dying cells. Considering ROCK-1 function in actin polymerization regulation, endosomal recycling and membrane blebbing, its direct association with PD-L1 and CMTM6 complex could serve as an additional therapeutic target with already successful PD-L1 therapies to avoid immune tolerance in cytotoxic tumors (Burr et al., 2017; Ren et al., 2019). If superior cell death could be maintained by keeping immune suppression in check, a potential new therapeutic avenue will open doors to give second lease of life to clinically tested DR5 agonist antibodies to enhance tumor immunity and overpower clinical efficacy.

TABLE 4
Exemplary Reagents Employed and Their Functions

| Reagent name | Type | Function |
|---|---|---|
| GSK269962A, also | Small molecule inhibitor | ROCK1 inhibitor |
| referred to as GSK269 | | |
| GSK429286A, also | Small molecule inhibitor | ROCK1 inhibitor |
| referred to as GSK429 | | |
| BMS202 | Small molecule inhibitor | PD-1 inhibitor |
| Lexatumumab (Lexa) | Monospecific Antibody | Human DR5 activator |
| AMG655 (AMG) | Monospecific Antibody | Human DR5 activator |
| Tigatuzumab (Tiga) | Monospecific Antibody | Human DR5 activator |
| KMTR2 | Monospecific Antibody | Human DR5 activator |
| MD5-1 | Monospecific Antibody | Murine DR5 activator |
| Avelumab-MD5-1 | Bispecific Antibody | Murine DR5 activator |
| Avelumab-MD5-1 | Bispecific Antibody | PD-L1 inhibitor |
| Avelumab | Bispecific Antibody | PD-L1 inhibitor |
| OKT3 | Monospecific Antibody | CD3 activator |

TABLE 5
Reagents and Suppliers

| Reagents, Antibodies etc. | Commercial Source | Catalog No. |
|---|---|---|
| PD-L1 (EIL3N ®) XP ® Rabbit mAb | Cell Signaling Technology | 13684 |
| CMTM6 Antibody | Cell Signaling Technology | 34557 |
| ROCK1 (C8F7) Rabbit mAb | Cell Signaling Technology | 4035 |
| α-Tubulin Antibody | Cell Signaling Technology | 2144 |
| Cleaved Caspase-3 Antibody | Cell Signaling Technology | 9661 |
| Anti-DR5 (Human) | Cell Signaling Technology | 3696 |
| GAPDH Antibody | Cell Signaling Technology | 5174 |
| GAPDH Antibody | Santa Cruz Biotechnology | Sc-32233 |
| Cleaved PARP antibody | Cell Signaling Technology | 9953 |
| Calreticulin | Cell Signaling Technology | 12238 |
| Mouse Anti-Human CD47 | BD Biosciences | 556044 |
| CSN5/COPS5 | Cell Signaling Technology | 6895 |
| P-p65 | Cell Signaling Technology | 3033 |
| p65 | Cell Signaling Technology | 6956 |
| Caspase-8 | Cell Signaling Technology | 9746 |
| P-MLC | Cell Signaling Technology | 3675 |
| E-Cadherin | Cell Signaling Technology | 4065 |
| E-Cadherin | Cell Signaling Technology | 14472 |
| N-Cadherin | Cell Signaling Technology | 13116 |
| Vimentin | Cell Signaling Technology | 5741 |
| P-STAT3 | Cell Signaling Technology | 9145 |
| STAT3 | Cell Signaling Technology | 12640 |
| ERK | Cell Signaling Technology | 9102 |
| Folate Receptor alpha Polyclonal | Invitrogen | PA5-24186 |
| Antibody (FOLR1) | | |
| FOLR1 | R & D System | MAB5646 |
| Anti-Rabbit-HRP antibody | Cell Signaling Technology | 7074 |
| Anti-Mouse-HRP antibody | Cell Signaling Technology | 7076 |
| PD-L1 Antibody | Novus Biologicals | NBP1-76769 |
| Commercial MD5-1 (anti-Murine | Abcam | ab171248 |
| DR5) antibody | | |
| CD8a Monoclonal Antibody (53-6.7), | Invitrogen | 12-0081-82 |
| PE, eBioscience ™ | | |
| CD4 Monoclonal Antibody (RM4-5), | Invitrogen | 17-0042-82 |
| APC, eBioscience ™ | | |
| Mouse CD25/IL-2R alpha Alexa | R & D Systems | FAB9164G |
| Fluor ® 488-conjugated Antibody | | |
| Cy5 conjugated Anti-Human IgG | Jackson ImmunoResearch | 709-175-149 |
| (H + L) | | |
| Alexa Fluor ® 488 AffiniPure Goat | Jackson ImmunoResearch | 111-545-003 |
| Anti-Rabbit IgG (H + L) | | |
| Cy ™5 AffiniPure Donkey Anti-Mouse | Jackson ImmunoResearch | 715-175-150 |
| IgG (H + L) | | |
| Ubiquitin polyclonal antibody | Enzolifesciences | ADI-SPA-200 |
| K48-linkage Specific Polyubiquitin | Cell Signaling Technology | 4289 |
| Antibody | | |
| Z-VAD-FMK | Apex Bio | A1902 |
| Z-DEVD-FMK | Apex Bio | A1920 |
| GSK 269962A | TargetMol | T3518 |
| GSK 429286 | Tocris | 3726 |
| Y27632 | Apex Bio | A3008 |
| Y39983 | Sclleckchem.com | S7935 |
| Doxorubicin | Cayman Chemical | 15007 |
| | Company | |
| Etoposide | Millipore | 341205 |
| MG-132 | Sigma | 474787 |
| Tunicamycin | MP Biomedicals | 150028 |
| Xenolight D- luciferin potassium salt | PerkinElmer | P/N 122799 |
| CHO free style Media | Thermo Fisher | 12651014 |
| HiTrap MabSelect Sure column | GE | 11003493 |
| Protein-A resin | Thermo Fisher | P153142 |
| HisPur Ni-NTA resin | Thermo Fisher | 88221 |
| HiPure Plasmid Maxiprep kit | Invitrogen | K21007 |
| Endpoint Chromogenic LAL endotoxin | Lonza | 50-648U |
| assay kit | | |
| AlamarBlue Cell viability reagent | Thermo Fisher | DAL1100 |
| MTT reagent | Thermo Fisher | V13154 |
| Infusion | Takara BioScience | STO344 |
| CHO CD efficient Feed B | | A1024001 |
| PEI transfection reagent | Thermo Fisher | BMS1003A |
| Matrigel | Corning | 354234 |
| Mouse anti-human IgG1 Fc | Thermo Fisher | A10648 |
| EZ-Link Sulfo-NHS-SS-Biotin | Thermo Fisher | 21331 |
| Ficoll ®-Paque PREMIUM 1.084 | GE Healthcare | 17-5446-02 |
| Corning ® 500 mL RPMI 1640 | Corning | 10-040-CV |
| Corning ® 500 mL DMEM (Dulbecco's | Corning | 10-13-CV |
| Modified Eagle's Medium) | | |
| VWR ® Cell Strainers, DNase/RNase | VWR | 10199-656 |
| Free, Non-Pyrogenic, Sterile | | |
| Halt protease inhibitor | Thermo Fisher | 78430 |
| AST reagent | Pointe Scientific | 23-666-1221 |
| EnzyChrom ALT Assay Kit | Bioassay Systems | EASTR-100 |
| Goat anti-Human IgG (H&L) Coated | Spherotech | HMS-30-10 |
| Magnetic Particles, Smooth | | |
| Surface | | |
| Superscript II | Invitrogen | 18064014 |

TABLE 6
List of Cell Lines Employed and Generated

| Species and Cell Line Name | Description or Source^{a} | Catalog Number^{a} |
|---|---|---|
| Human: OVCAR-3 | Ovarian adenocarcinoma | ATCC HTB-161 |
| Human: MDA-MB-436 | TNBC | ATCC HTB-130 |
| Human: MDA-MB-231 | TNBC | ATCC HTB-26 |
| Human: MDA-MB-231-2B | TNBC | ATCC HTB-26 |
| Human: PD-1 Effector Cells | Promega | J1151 |
| Human: PD-L1 aAPC/CHO- | Promega | J1091 |
| K1 Cells | | |
| Human: A549 | Lung cancer | ATCC CLL-185 |
| Human: Cavo-3 | Ovarian cancer | ATCC HTB-75 |
| Human: HCC1806 | TNBC | ATCC CRL-2335 |
| Human: PANK1 | Pancreatic cancer | ATCC CRL-1469 |
| Human: U87 | Brain cancer | ATCC HTB-14 |
| Human: HCT116 | Colon cancer | ATCC CCL247 |
| Human: Colo-205 | Colon cancer | ATCC CCL-222 |
| Human: CHO-K cells | Stably transformed as | ATCC CCL-61 |
| | described herein | |
| Mouse: 4T1 | Murine TNBC cell | ATCC CRL-2539 |
| Mouse: MC38 | Murine colon cancer | CVCL_B288; Corbett et al., |
| | | 1975 |
| Mouse: ID8 | Murine ovarian cancer | ABC-TC3940; AcceGen |
| PDX cell line: UCD52 | Mouse liver metastasis | Wright Center |
| Mouse: MC38 Chimeric | Internally generated | Human DR5 expressing |
| human-mouse DR-5 G4S | | murine cells |
| (Chi-G4S-DR5 cells) | | |
| Mouse: 4T1 Chimeric human- | Internally generated | Human DR5 expressing |
| mouse DR5 G4S (Chi- | | murine cells |
| G4S-DR5 cells) | | |
| Mouse: 4T1 Chimeric human- | Internally generated | Human DR5 expressing |
| mouse DR5 no G4S | | murine cells |
| linker (Chi-DR5 cells) | | |
| Mouse: MC38 Chimeric | Internally generated | Human DR5 expressing |
| human-mouse DR5 no | | murine cells |
| G4S linker (Chi-DR5 | | |
| cells) | | |
| Mouse: 4T1 complete human | Internally generated | Human DR5 expressing |
| DR5 no G4S linker | | murine cells |
| (huDR5 cells) | | |
| Human: MDA-MB-436 | Internally generated | DR5 knockout cells |
| DR5Ko | | |
| Human: MDA-MB-231 | Internally generated | DR5 knockout cells |
| DR5Ko | | |
| Human: MDA-MB-231-2B | Internally generated | DR5 knockout cells |
| DR5Ko | | |
| Human: MDA-MB-436 DR5 | Internally generated | DR5 resistant Cells |
| antibody Resistant | | |
| Human: MDA-MB-231 DR-5 | Internally generated | DR5 resistant Cells |
| antibody Resistant | | |
| Human: OVCAR3 DR-5 | Internally generated | DR5 resistant Cells |
| antibody Resistant | | |

^{a}List of Abbreviations: Triple-negative breast cancer (TNBC); Promega Corporation, Madison, Wisconsin, United States of America (Promega); ATCC ®, Manassas, Virginia, United States of America (ATCC); AcceGen, Fairfield, New Jersey, United States of America (AcceGen); Wright Center for Clinical and Translational Research Bioinformatics Core, Virginia Commonwealth University, Richmond, Virginia, United States of America (Wright Center).

TABLE 7
Amino Acid Sequences of DR5 Agonists and Other Control Antibodies

| | |
|---|---|
| Lexatumumab lambda (V_{L}) | SEQ ID NO: 1 |
| Lexatumumab IgG1 (V_{H}) | SEQ ID NO: 2 |
| Tigatuzumab c-kappa (V_{L}) | SEQ ID NO: 3 |
| Tigatuzumab IgG1 (V_{H}) | SEQ ID NO: 4 |
| AMG-655 (Conatumumab) c-kappa (V_{L}) | SEQ ID NO: 5 |
| AMG-655 (Conatumumab) IgG1 (V_{H}) | SEQ ID NO: 6 |
| KMTR2 c-kappa (V_{L}) | SEQ ID NO: 7 |
| KMTR2 IgG1 (V_{H}) | SEQ ID NO: 8 |
| Avelumab c-kappa (V_{L}) | SEQ ID NO: 9 |
| Avelumab IgG4 (V_{H}) | SEQ ID NO: 10 |
| Farletuzumab c-kappa (V_{L}) | SEQ ID NO: 11 |
| Farletuzumab IgG1 (V_{H}) | SEQ ID NO: 12 |

Lexatumumab lambda (V_{L})-SEQ ID NO: 1
SSELTQDPAVSVALGQTVRITCQGDSLRSYYASWYQQKPGQAPVLVIYGKNNRPSGIPDR
FSGSSSGNTASLTITGAQAEDEADYYCNSRDSSGNHVVFGGGTKLTVLGQPKAAPSVTLFP
PSSEELQANKATLVCLISDFYPGAVTVAWKADSSPVKAGVETTTPSKQSNNKYAASSYLS
LTPEQWKSHRSYSCQVTHEGSTVEKTVAPTECS
Lexatumumab IgG1 (V_{H})-SEQ ID NO: 2
EVQLVQSGGGVERPGGSLRLSCAASGFTFDDYGMSWVRQAPGKGLEWVSGINWNGGST
GYADSVKGRVTISRDNAKNSLYLQMNSLRAEDTAVYYCAKILGAGRGWYFDLWGKGTT
VTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKTHTCPPCPAPEA
AGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
Tigatuzumab c-kappa (V_{L})-SEQ ID NO: 3
DIQMTQSPSSLSASVGDRVTITCKASQDVGTAVAWYQQKPGKAPKLLIYWASTRHTGVPS
RFSGSGSGTDFTLTISSLQPEDFATYYCQQYSSYRTFGQGTKVEIKRTVAAPSVFIFPPSDEQ
LKSGTASVVCLLNNFYPREAKVQWKVDNALQSGNSQESVTEQDSKDSTYSLSSTLTLSKA
DYEKHKVYACEVTHQGLSSPVTKSFNRGEC
Tigatuzumab IgG1 (V_{H})-SEQ ID NO: 4
EVQLVESGGGLVQPGGSLRLSCAASGFTFSSYVMSWVRQAPGKGLEWVATISSGGSYTY
YPDSVKGRFTISRDNAKNTLYLQMNSLRAEDTAVYYCARRGDSMITTDYWGQGTLVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPEAAGGP
SVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQ
QGNVFSCSVMHEALHNHYTQKSLSLSPGK
AMG-655 (Conatumumab) c-kappa (V_{L})-SEQ ID NO: 5
EIVLTQSPGTLSLSPGERATLSCRASQGISRSYLAWYQQKPGQAPSLLIYGASSRATGIPDRF
SGSGSGTDFTLTISRLEPEDFAVYYCQQFGSSPWTFGQGTKVEIKRTVAAPSVFIFPPSDEQ
LKSGTASVVCLLNNFYPREAKVQWKVDNALQSGNSQESVTEQDSKDSTYSLSSTLTLSKA
DYEKHKVYACEVTHQGLSSPVTKSFNRGEC
AMG-655 (Conatumumab) IgG1 (V_{H})-SEQ ID NO: 6
EVQLLESGGGLVQPGGSLRLSCAASGFTFSSYIMMWVRQAPGKGLEWVSSIYPSGGITFY
ADTVKGRFTISRDNSKNTLYLQMNSLRAEDTAVYYCARIKLGTVTTVDYWGQGTLVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVESKYGPPCPSCPAPEFLGGPSVFL
FPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKN
QVSLTCLVKGFYPSDIA VEWESNGQPENNYKTTPPVLDSDGSFFLYSKLTVDKSRWQEGN
VFSCSVMHEALHNHYTQKSLSLSPGK
KMTR2 c-kappa (V_{L})-SEQ ID NO: 7
EIVLTQSPATLSLSPGERATLSCRASQSVSSYLAWYQQKPGQAPRLLIYDASNRATGIPARF
SGSGSGTDFTLTISSLEPEDFAVYYCQQRSNWPLTFGGGTKVEIKRTVAAPSVFIFPPSDEQ
LKSGTASVVCLLNNFYPREAKVQWKVDNALQSGNSQESVTEQDSKDSTYSLSSTLTLSKA
DYEKHKVYACEVTHQGLSSPVTKSFNRGEC
KMTR2 IgG1 (V_{H})-SEQ ID NO: 8
QVQLVQSGAEMKKPGASVKVSCKTSGYTFTNYKINWVRQAPGQGLEWMGWMNPDTDS
TGYPQKFQGRVTMTRNTSISTAYMELSSLRSEDTAVYYCARSYGSGSYYRDYYYGMDV
WGQGTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
Avelumab c-kappa (V_{L})-SEQ ID NO: 9
MGWSCIILFLVATATGVHSQSALTQPASVSGSPGQSITISCTGTSSDVGGYNYVSWYQQHP
GKAPKLMIYDVSNRPSGVSNRFSGSKSGNTASLTISGLQAEDEADYYCSSYTSSSTRVFGT
GTKVTVLRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
Avelumab IgG4 (V_{H})-SEQ ID NO: 10
MGWSCIILFLVATATGVHSEVQLLESGGGLVQPGGSLRLSCAASGFTFSSYIMMWVRQAP
GKGLEWVSSIYPSGGITFYADTVKGRFTISRDNSKNTLYLQMNSLRAEDTAVYYCARIKL
GTVTTVDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVESKY
GPPCPSCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGS
FFLYSKLTVDKSRWQEGNVFSCSVMHEALHNHYTQKSLSLSLG
Farletuzumab c-kappa (V_{L})-SEQ ID NO: 11
DIQLTQSPSSLSASVGDRVTITCSVSSSISSNNLHWYQQKPGKAPKPWIYGTSNLASGVPSR
FSGSGSGTDYTFTISSLQPEDIATYYCQQWSSYPYMYTFGQGTKVEIKRTVAAPSVFIFPPS
DEQLKSGTASVVCLLNNFYPREAKVQWKVDNALQSGNSQESVTEQDSKDSTYSLSSTLTL
SKADYEKHKVYACEVTHQGLSSPVTKSFNRGEC
Farletuzumab IgG1 (V_{H})-SEQ ID NO: 12
EVQLVESGGGVVQPGRSLRLSCSASGFTFSGYGLSWVRQAPGKGLEWVAMISSGGSYTY
YADSVKGRFAISRDNAKNTLFLQMDSLRPEDTGVYFCARHGDDPAWFAYWGQGTPVTV
SSASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAAGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSLPGK

TABLE 8
Amino Acid Sequences of huDR5 and Chimeric DR5 Proteins
Expressed in Murine 4T1, ID8, and MC38 Cells

| | |
|---|---|
| HuDR5 | SEQ ID NO: 13 |
| Chi-DR5 | SEQ ID NO: 14 |
| Chi-G4S DR5 | SEQ ID NO: 15 |

HuDR5-SEQ ID NO: 13
MSEQRGQNAPAASGARKRHGPGPREARGARPGPRVPKTLVLVVAAVLLLVSAESALITQ
QDLAPQQRAAPQQKRSSPSEGLCPPGHHISEDGRDCISCKYGQDYSTHWNDLLFCLRCTR
CDSGEVELSPCTTTRNTVCQCEEGTFREEDSPEMCRKCRTGCPRGMVKVGDCTPWSDIEC
VHKESGTKHSGEVPAVEETVTSSPGTPASPCSLSGIIIGVTVAAVVLIVAVFVCKSLLWKKV
LPYLKGICSGGGGDPERVDRSSQRPGAEDNVLNEIVSILQPTQVPEQEMEVQEPAEPTGVN
MLSPGESEHLLEPAEAERSQRRRLLVPANEGDPTETLRQCFDDFADLVPFDSWEPLMRKL
GLMDNEIKVAKAEAAGHRDTLYTMLIKWVNKTGRDASVHTLLDALETLGERLAKQKIED
HLLSSGKFMYLEGNADSAMS
Chi-DR5-SEQ ID NO: 14
MSEQRGQNAPAASGARKRHGPGPREARGARPGPRVPKTLVLVVAAVLLLVSAESALITQ
QDLAPQQRAAPQQKRSSPSEGLCPPGHHISEDGRDCISCKYGQDYSTHWNDLLFCLRCTR
CDSGEVELSPCTTTRNTVCQCEEGTFREEDSPEMCRKCRTGCPRGMVKVGDCTPWSDIEC
VHKESGTKHSGEVPAVEETVTSSPGTPASPCSLSGLWIGLLVPVVLLIGALLVWKTGAWR
QWLLCIKRGCERDPESANSVHLSLLDRQTSSTTNDSNHNTEPGKTQKTGKKLLVPVNGND
SADDLKFIFEYCSDIVPFDSWNRLMRQLGLTDNQIQMVKAETLVTREALYQMLLKWRHQ
TGRSASINHLLDALEAVEERDAMEKIEDYAVKSGRFTYQNAAAQPETGPGGSQCV
Chi-G4S DR5-SEQ ID NO: 15
MEPPGPSTPTASAAARADHYTPGLRPLPKRRLLYSFALLLAVLQAVFVPVTAITQQDLAPQ
QRAAPQQKRSSPSEGLCPPGHHISEDGRDCISCKYGQDYSTHWNDLLFCLRCTRCDSGEV
ELSPCTTTRNTVCQCEEGTFREEDSPEMCRKCRTGCPRGMVKVGDCTPWSDIECVHKESG
TKHSGEVPAVEETVTSSPGTPASPCSGGGGSLGLWIGLLVPVVLLIGALLVWKTGAWRQW
LLCIKRGCERDPESANSVHLSLLDRQTSSTTNDSNHNTEPGKTQKTGKKLLVPVNGNDSA
DDLKFIFEYCSDIVPFDSWNRLMRQLGLTDNQIQMVKAETLVTREALYQMLLKWRHQTG
RSASINHLLDALEAVEERDAMEKIEDYAVKSGRFTYQNAAAQPETGPGGSQCV

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Agata et al. (1996) Expression of the PD-1 antigen on the surface of stimulated mouse T and B lymphocytes. Int Immunol 8:765-772.
Aktas et al. (2019) Atezolizumab and Nab-Paclitaxel in Advanced Triple-Negative Breast Cancer. N Engl J Med 380:985-986.
Altschul et al. (1990a) Basic local alignment search tool. J Mol Biol 215:403-410.
Altschul et al. (1990b) Protein database searches for multiple alignments. Proc Natl Acad Sci USA 87:14:5509-5513.
Altschul et al. (1997) Gapped BLAST and PSI-BLAST: a new generation of protein database search programs. Nucleic Acids Res 25:3389-3402.
Alzubi et al (2019) Separation of breast cancer and organ microenvironment transcriptomes in metastases. Breast Cancer Res 21:36.
Anderson et al. (2017) Obstacles Posed by the Tumor Microenvironment to T cell Activity: A Case for Synergistic Therapies. Cancer Cell 31:311-325.
Ashkenazi & Herbst (2008) To kill a tumor cell: the potential of proapoptotic receptor agonists. J Clin Invest 118:1979-1990.
Ashkenazi (2008) Directing cancer cells to self-destruct with pro-apoptotic receptor agonists. Nat Rev Drug Discov 7:1001-1012.
Ashkenazi (2015) Targeting the extrinsic apoptotic pathway in cancer: lessons learned and future directions. J Clin Invest 125:487-489.
Asiedu et al. (2011) TGFbeta/TNF(alpha)-mediated epithelial-mesenchymal transition generates breast cancer stem cells with a claudin-low phenotype. Cancer Res 71:4707-4719.
Baker & Masters (2018) Caspase substrates won't be defined by a four-letter code. J Biol Chem 293:7068-7069.
Barbas (1995) Synthetic human antibodies. Nature Medicine 1:837-839.
Bennett et al. (2003) Program Death-1 Engagement Upon TCR Activation Has Distinct Effects on Costimulation and Cytokine-Driven Proliferation: Attenuation of ICOS, IL-4, and IL-21, But Not CD28, IL-7, and IL-15 Responses. J Immunol 170:711-718.
Binnewies et al. (2018) Understanding the tumor immune microenvironment (TIME) for effective therapy. Nat Med 24:541-550.
Bird et al. (1988) Single-chain antigen-binding proteins. Science 242:423-426.
Brennan et al. (1985) Preparation of Bispecific Antibodies by Chemical Recombination of Monoclonal Immunoglobulin G1 Fragments. Science 229:81-83.
Brinkmann & Kontermann (2017) The making of bispecific antibodies. MAbs 9:182-212.
Burr et al. (2017) CMTM6 maintains the expression of PD-L1 and regulates anti-tumour immunity. Nature 549:101-105.
Burton & Barbas (1994) Human antibodies from combinatorial libraries. Adv Immunol 57:191-280.
Calve et al. (2016) Incorporation ofnon-canonical amino acids into the developing murine proteome. Sci Rep 6:32377.
Camidge (2008) Apomab: an agonist monoclonal antibody directed against Death Receptor 5/TRAIL-Receptor 2 for use in the treatment of solid tumors. Expert Opin Biol Ther 8:1167-1176.
Carter et al. (1992a) Humanization of an anti-p185HER2 antibody for human cancer therapy. Proc Natl Acad Sci USA 89:4285-4289.
Carter et al. (1992b) High level Escherichia coli expression and production of a bivalent humanized antibody fragment. Bio/Technology 10:163-167.
Caruso & Poon (2018) Apoptotic Cell-Derived Extracellular Vesicles: More Than Just Debris. Front Immunol 9:1486.
Chan et al. (2019) IL-6/JAK1 pathway drives PD-L1 Y112 phosphorylation to promote cancer immune evasion. J Clin Invest 129:3324-3338.
Chen et al. (2018) Exosomal PD-L1 contributes to immunosuppression and is associated with anti-PD-1 response. Nature 560:382-386.
Chin et al. (2015) Rho-associated kinase signalling and the cancer microenvironment: novel biological implications and therapeutic opportunities. Expert Rev Mol Med 17:e17.
Chothia & Lesk (1987) Canonical structures for the hypervariable regions of immunoglobulins. J Mol Biol 196:901-917.
Chou & Fasman (1974) Prediction of protein conformation. Biochemistry 13:222-245.
Chou & Fasman (1978) Empirical Predictions of Protein Conformation. Annual Review of Biochemistry 47:251-276.
Chou & Fasman (1979) Prediction of beta-turns. Biophysical Journal 26:367-383.
Chun et al. (2002) Pleiotropic defects in lymphocyte activation caused by caspase-8 mutations lead to human immunodeficiency. Nature 419:395-399.
Cohen (2005) Caspase inactivation of the proteasome. Cell Death Differ 12:1218.
Cole et al. (1985) “The EBV-hybridoma technique and its application to human lung cancer” in Monoclonal Antibodies in Cancer Therapy, Allen R. Liss, Inc., New York, New York, United States of America, pages 77-96.
Coleman et al. (2001) Membrane blebbing during apoptosis results from caspase-mediated activation of ROCK I. Nat Cell Biol 3:339-345.
Condamine et al. (2014) ER stress regulates myeloid-derived suppressor cell fate through TRAIL-R-mediated apoptosis. J Clin Invest 124:2626-2639.
Corbett et al. (1975) Tumor induction relationships in development of transplantable cancers of the colon in mice for chemotherapy assays, with a note on carcinogen structure. Cancer Res 35:2434-2439.
Cote et al. (1983) Generation of human monoclonal antibodies reactive with cellular antigens. Proc Natl Acad Sci USA 80:2026-2030.
Croce & Reed (2016) Finally, An Apoptosis-Targeting Therapeutic for Cancer. Cancer Res 76:5914-5920.
de Kruif et al. (1995) Selection and application of human single chain Fv antibody fragments from a semi-synthetic phage antibody display library with designed CDR3 regions. J Mol Biol 248:97-105.
Deutscher et al. (1990) Guide to Protein Purification, Harcourt Brace Jovanovich, San Diego, California, United States of America.
Devereux et al. (1984) A comprehensive set of sequence analysis programs for the VAX. Nucl Acids Res 12:387.
Durocher & Butler (2009) Expression systems for therapeutic glycoprotein production. Curr Opin Biotechnol 20:700-707.
Ferrari et al. (1998) Differential regulation and ATP requirement for caspase-8 and caspase-3 activation during CD95- and anticancer drug-induced apoptosis. J Exp Med 188:979-984.
Forero-Torres et al. (2010) Phase I trial of weekly tigatuzumab, an agonistic humanized monoclonal antibody targeting death receptor 5 (DR5). Cancer Biother Radiopharm 25:13-19.
Forero-Torres et al. (2013) Phase 2, multicenter, open-label study of tigatuzumab (CS-1008), a humanized monoclonal antibody targeting death receptor 5, in combination with gemcitabine in chemotherapy-naive patients with unresectable or metastatic pancreatic cancer. Cancer Med 2(6):925-932.
Forero-Torres et al. (2015) TBCRC 019: A Phase II Trial ofNanoparticle Albumin-Bound Paclitaxel with or without the Anti-Death Receptor 5 Monoclonal Antibody Tigatuzumab in Patients with Triple-Negative Breast Cancer. Clin Cancer Res 21:2722-2729.
Genaro (1985) Remington's Pharmaceutical Sciences, Mack Publishing Co., Easton, Pennsylvania, United States of America.
Graves et al. (2014) Apo2L/TRAIL and the death receptor 5 agonist antibody AMG 655 cooperate to promote receptor clustering and antitumor activity. Cancer Cell 26:177-189.
Green & Sambrook (2012) Molecular Cloning: A Laboratory Manual, 4^{th} Edition. Cold Spring Harbor Laboratory Press, Cold Spring Harbor, New York, United States of America.
Gregory & Dransfield (2018) Apoptotic Tumor Cell-Derived Extracellular Vesicles as Important Regulators of the Onco-Regenerative Niche. Front Immunol 9:1111.
Griffiths-Jones (2004) The microRNA Registry. Nucleic Acids Research 32(suppl_1):D109-D111.
Gross & Mienhofer (1981) The Peptides, Vol. 3, Academic Press, New York, New York, United States of America, pp. 3-88.
Gruber et al. (1994) Efficient tumor cell lysis mediated by a bispecific single chain antibody expressed in Escherichia coli. J Immunol 152:5368.
Gu et al. (1997) Construction and expression of mouse-human chimeric antibody SZ-51 specific for activated platelet P-selectin. Thrombosis and Haemostasis 77(4):755-759.
Guisier et al. (2019) A rationale for surgical debulking to improve anti-PD1 therapy outcome in non small cell lung cancer. Sci Rep 9:16902.
Haanen (2017) Converting Cold into Hot Tumors by Combining Immunotherapies. Cell 170:1055-1056.
Harlow & Lane (1988) Antibodies: A Laboratory Manual. Cold Spring Harbor Laboratory Press, Cold Spring Harbor, New York, United States of America.
Hsu et al. (2018) Posttranslational Modifications of PD-L1 and Their Applications in Cancer Therapy. Cancer Res 78:6349-6353.
Hughes et al. (2016) Co-operative and Hierarchical Binding of c-FLIP and Caspase-8: A Unified Model Defines How c-FLIP Isoforms Differentially Control Cell Fate. Mol Cell 61:834-849.
Huse et al. (1989) Generation of a large combinatorial library of the immunoglobulin repertoire in phage lambda. Science 246:1275-1281.
Huston et al. (1988) Protein engineering of antibody binding sites: recovery of specific activity in an anti-digoxin single-chain Fv analogue produced in Escherichia coli. Proc Natl Acad Sci USA 85:5879.
Jones et al (1986) Replacing the complementarity-determining regions in a human antibody with those from a mouse. Nature 321:522.
Kalthoff & Trauzold (2009) Following the death TRAIL to hunt down tumor cells: translating programmed cell death signaling mechanisms into clinical practice. Results Probl Cell Differ 49:vii-xiii.
Kaplan-Lefko et al. (2010) Conatumumab, a fully human agonist antibody to death receptor 5, induces apoptosis via caspase activation in multiple tumor types. Cancer Biol Ther 9:618-631.
Karlin & Altschul (1990) Methods for assessing the statistical significance of molecular sequence features by using general scoring schemes. Proc Natl Acad Sci USA 87(6):2264-2268.
Karlin & Altschul (1993) Applications and statistics for multiple high-scoring segments in molecular sequences. Proc Natl Acad Sci USA 90(12):5873-5877.
Kohler & Milstein (1975) Continuous cultures of fused cells secreting antibody of predefined specificity. Nature 256(5517):495-497.
Kozbor & Roder (1983) The production of monoclonal antibodies from human lymphocytes. Immunol Today 4(3):72-79.
Kyte & Doolittle (1982) A simple method for displaying the hydropathic character of a protein. J Mol Biol 157(1):105-132.
LeBlanc et al. (2002) Tumor-cell resistance to death receptor—induced apoptosis through mutational inactivation of the proapoptotic Bcl-2 homolog Bax. Nat Med 8:274-281.
Lee et al. (2019) Removal ofN-Linked Glycosylation Enhances PD-L1 Detection and Predicts Anti-PD-1/PD-L1 Therapeutic Efficacy. Cancer Cell 36:168-178 e164.
Leelatian et al. (2017) Preparing Viable Single Cells from Human Tissue and Tumors for Cytomic Analysis. Curr Protoc Mol Biol 118:25C.21.21-25C.21.23.
Li & Ravetch (2012) Apoptotic and antitumor activity of death receptor antibodies require inhibitory Fcgamma receptor engagement. Proc Natl Acad Sci USA 109:10966-10971.
Li et al. (2007) Antitumor efficacy of LBYl35, an anti-DR5 monoclonal antibody, alone or in combination with chemotherapy in human colon tumor cell lines and xenografts. AACR Meeting Abstracts 2007. Abstract 4874.
Lim et al. (2016) Deubiquitination and Stabilization of PD-L1 by CSN5. Cancer Cell 30:925-939.
Madore et al. (2016) PD-L1 Negative Status is Associated with Lower Mutation Burden, Differential Expression of Immune-Related Genes, and Worse Survival in Stage III Melanoma. Clin Cancer Res 22:3915-3923.
Marini (2006) Drug evaluation: lexatumumab, an intravenous human agonistic mAb targeting TRAIL receptor 2. Curr Opin Mol Ther 8:539-546.
Marks et al. (1991) By-passing immunization. Human antibodies from V-gene libraries displayed on phage. J Mol Biol 222:581-597.
Mavratzas et al. (2020) Atezolizumab for use in PD-L1-positive unresectable, locally advanced or metastatic triple-negative breast cancer. Future Oncol 16:4439-4453.
Melero et al. (2014) T-cell and NK-cell infiltration into solid tumors: a key limiting factor for efficacious cancer immunotherapy. Cancer Discov 4:522-526.
Mellman et al. (2011) Cancer immunotherapy comes of age. Nature 480:480-489.
Milstein & Cuello (1983) Hybrid hybridomas and their use in immunohistochemistry Nature 305:537.
Miura (2012) Apoptotic and nonapoptotic caspase functions in animal development. Cold Spring Harb Perspect Biol 4(10):a008664.
Morimoto & Inouye (1992) Single-step purification of F(ab′)₂ fragments of mouse monoclonal antibodies (immunoglobulins G) by hydrophobic interaction high performance liquid chromatography using TSKgel Phenyl-5PW. J Biochem Biophys Methods 24(1-2):107-117.
Morrison et al. (1984) Chimeric human antibody molecules: mouse antigen-binding domains with human constant region domains. Proc Natl Acad Sci USA 81(21):6851-6855.
Motoki et al. (2005) Enhanced apoptosis and tumor regression induced by a direct agonist antibody to tumor necrosis factor-related apoptosis-inducing ligand receptor 2. Clin Cancer Res 11:3126-3135.
Nam et al. (2018) Combined Rho-kinase inhibition and immunogenic cell death triggers and propagates immunity against cancer. Nat Commun 9:2165.
Narayan & Vaughn (2015) Pharmacokinetic and toxicity considerations in the use of neoadjuvant chemotherapy for bladder cancer. Expert Opin Drug Metab Toxicol 11:731-742.
Neuberger et al. (1984) Recombinant antibodies possessing novel effector functions. Nature 312(5995):604-608.
Newton et al. (2019) Activity of caspase-8 determines plasticity between cell death pathways. Nature 575:679-682.
Nolan et al. (2017) Combined immune checkpoint blockade as a therapeutic strategy for BRCA1-mutated breast cancer. Sci Transl Med 9(393):eaal4922.
Obeid et al. (2007) Calreticulin exposure dictates the immunogenicity of cancer cell death. Nat Med 13:54-61.
Okazaki et al. (2002) New regulatory co-receptors: inducible co-stimulator and PD-1. Curr Opin Immunol 14:779-782.
Opzoomer et al. (2019) Cytotoxic Chemotherapy as an Immune Stimulus: A Molecular Perspective on Turning Up the Immunological Heat on Cancer. Front Immunol 10:1654.
Pardoll (2012) The blockade of immune checkpoints in cancer immunotherapy. Nat Rev Cancer 12:252-264.
PCT International Patent Application Publication Nos. WO 1992/02190, WO 1993/16185, WO 2013/079174.
Ponder & Boise (2019) The prodomain of caspase-3 regulates its own removal and caspase activation. Cell Death Discov 5:56.
Presta (1992) Antibody engineering. Curr Opin Biotechnol 3(4):394-398.
Presta et al. (1993) Humanization of an antibody directed against IgE. J Immunol 151(5):2623-2632.
Ren et al. (2019) Osteosarcoma cell intrinsic PD-L2 signals promote invasion and metastasis via the RhoA-ROCK-LIMK2 and autophagy pathways. Cell Death Dis 10:261.
Riechmann et al. (1988) Reshaping human antibodies for therapy. Nature 332(6162):323-327.
Roumenina et al. (2019) Tumor Cells Hijack Macrophage-Produced Complement C1q to Promote Tumor Growth. Cancer Immunol Res 7:1091-1105.
Salmena et al. (2003) Essential role for caspase 8 in T-cell homeostasis and T-cell-mediated immunity. Genes Dev 17:883-895.
Saunders (2019) Conceptual Approaches to Modulating Antibody Effector Functions and Circulation Half-Life. Front Immunol 10:1296.
Shen et al. (2011) Biogenesis of the posterior pole is mediated by the exosome/microvesicle protein-sorting pathway. J Biol Chem 286:44162-44176.
Shevach (2004) Fatal attraction: tumors beckon regulatory T cells. Nat Med 10:900-901.
Shivange et al. (2018) A Single-Agent Dual-Specificity Targeting of FOLR1 and DR5 as an Effective Strategy for Ovarian Cancer. Cancer Cell 34:331-345 e311.
Sims et al. (1993) A humanized CD18 antibody can block function without cell destruction. J Immunol 151(4):2296-2308.
Solier et al. (2017) Non-apoptotic functions of caspases in myeloid cell differentiation. Cell Death Differ 24:1337-1347.
Spencer et al. (2009) Non-genetic origins of cell-to-cell variability in TRAIL-induced apoptosis.
Nature 459:428-432.
Sun et al. (2004) Caspase activation inhibits proteasome function during apoptosis. Mol Cell 14:81-93.
Sun et al. (2017) A systematic analysis of FDA-approved anticancer drugs. BMC Syst Biol 11:87.
Takeda et al. (1985) Construction of chimaeric processed immunoglobulin genes containing mouse variable and human constant region sequences. Nature 314(6010):452-454.
Takeda et al (2008) Death receptor 5 mediated-apoptosis contributes to cholestatic liver disease. Proc Natl Acad Sci USA 105:10895-10900.
Tamada et al. (2015) TRAIL-R2 Superoligomerization Induced by Human Monoclonal Agonistic Antibody KMTR2. Sci Rep 5:17936.
Tan & Lei (2019) Isolation of Tumor-Infiltrating Lymphocytes by Ficoll-Paque Density Gradient Centrifugation. Methods Mol Biol 1960:93-99.
Tuszynski et al. (1988) Thrombospondin promotes platelet aggregation. Blood 72:109-115.
U.S. Patent Application Publication Nos. 2002/0034765; 2003/0017534; 2003/0022244; 2003/0153043; 2004/0253645; 2006/0073137; 2018/0298087; 2018/0312588; 2018/0346564; 2019/0151448; 2020/0155521.
U.S. Pat. Nos. 4,554,101; 4,816,567; 4,946,778; 4,975,369; 5,001,065; 5,075,431; 5,081,235; 5,169,939; 5,202,238; 5,204,244; 5,225,539; 5,231,026; 5,292,867; 5,354,847; 5,436,157; 5,472,693; 5,482,856; 5,491,088; 5,500,362; 5,502,167; 5,530,101; 5,571,894; 5,585,089; 5,587,458; 5,641,870; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,479,284; 6,506,559; 6,548,640; 6,632,927; 6,639,055; 6,677,436; 6,750,325; 6,797,492; 7,056,704; 7,060,808; 7,592,441; 7,825,229; 7,825,230; 7,906,625; 7,960,359; 8,372,968; 8,398,980; 8,420,391; 8,436,150; 8,796,439; 10,253,111. 10,441,655; 10,684,287.
Usary et al. (2016) Overview of Genetically Engineered Mouse Models of Distinct Breast Cancer Subtypes. Curr Protoc Pharmacol 72:14.38.11-14.38.11.
Verhoeyen et al. (1988) Reshaping human antibodies: grafting an antilysozyme activity. Science 239(4847):1534-1536.
Vonderheide (2015) CD47 blockade as another immune checkpoint therapy for cancer. Nat Med 21:1122-1123.
Wajant (2019) Molecular Mode of Action of TRAIL Receptor Agonists-Common Principles and Their Translational Exploitation. Cancers (Basel) 11(7):954.
Wang et al. (2008) Characterization of a novel anti-DR5 monoclonal antibody WD1 with the potential to induce tumor cell apoptosis. Cell Mol Immunol 5:55-60.
Wang et al. (2014) The molecular mechanisms of TRAIL resistance in cancer cells: help in designing new drugs. Curr Pharm Des 20:6714-22.
Wang et al. (2017a) The CD4/CD8 ratio of tumor-infiltrating lymphocytes at the tumor-host interface has prognostic value in triple-negative breast cancer. Hum Pathol 69:110-117.
Wang et al. (2017b) Development of a robust reporter gene assay to measure the bioactivity of anti-PD-1/anti-PD-L1 therapeutic antibodies. J Pharm Biomed Anal 145:447-453.
Whitford et al. (1990) Flow cytometric analysis of tumor infiltrating lymphocytes in breast cancer.
Br J Cancer 62:971-975.
Wilson et al (2011) An Fcγ receptor-dependent mechanism drives antibody-mediated target-receptor signaling in cancer cells. Cancer Cell 19:101-113.
Winter & Milstein (1991) Man-made antibodies. Nature 349(6307):293-299.
Wollebo et al. (2013) Lentiviral transduction of neuronal cells. Methods Mol Biol 1078:141-146.
Wright et al. (1992) Genetically engineered antibodies: progress and prospects. Critical Reviews in Immunology 12(3,4):125-168.
Wu et al. (1997) KILLER/DR5 is a DNA damage-inducible p53-regulated death receptor gene. Nat Genet 17:141-143.
Wu et al. (2005) Selection for TRAIL resistance results in melanoma cells with high proliferative potential. FEBS Lett 579:1940-1944.
Zhang et al. (2007) Lexatumumab (TRAIL-receptor 2 mAb) induces expression of DR5 and promotes apoptosis in primary and metastatic renal cell carcinoma in a mouse orthotopic model. Cancer Lett 251:146-157.
Zhang et al (2017) Structural basis of the therapeutic anti-PD-L1 antibody atezolizumab. Oncotarget 8:90215-90224.
Zhang et al. (2020) Checkpoint therapeutic target database (CKTTD): the first comprehensive database for checkpoint targets and their modulators in cancer immunotherapy. J Immunother Cancer 8:e001247.

It will be understood that various details of the presently disclosed subject matter can be changed without departing from the scope of the presently disclosed subject matter. Furthermore, the foregoing description is for the purpose of illustration only, and not for the purpose of limitation.

1. A method for treating a tumor and/or a cancer in a subject in need thereof, the method comprising administering to the subject:
(a) a composition comprising an effective amount of an inhibitor of a Rho-Associated Coiled-Coil Containing Protein Kinase 1 (ROCK1) biological activity and/or an effective amount of a checkpoint inhibitor, optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor, or any combination thereof; and
(b) a composition comprising an effective amount of a Death Receptor 5 (DR5) agonist.

2. The method of claim 1, wherein the cancer comprises a solid tumor, optionally a solid tumor selected from the group consisting of an ovarian tumor, a glioblastoma, a pancreatic tumor, a lung tumor, and a triple negative breast (TNBC) tumor.

3. The method of claim 1, wherein the inhibitor of ROCK1 activity is a small molecule inhibitor.

4. The method of claim 1, wherein the inhibitor of ROCK 1 activity is selected from the group comprising N-(3-{[2-(4-Amino-1,2,5-oxadiazol-3-yl)-I-ethyl-IH-imidazo[4,5-c]pyridin-6-yl]oxy}phenyl)-4-[2-(morpholin-4-yl)ethoxy]benzamide (GSK269) and N-(6-Fluoro-1H-indazol-5-yl)-6-methyl-2-oxo-4-[4-(trifhioromethyl)phenyl]-3,4-dihydro-IH-pyridine-5-carboxamide (GSK429).

5. The method of claim 1, wherein the checkpoint inhibitor comprises an antibody, optionally an antibody that binds to a CTLA4 polypeptide, a PD-1 polypeptide, and/or a PD-L1 polypeptide.

6. The method of claim 5, wherein the antibody is selected from the group consisting of avelumab, atezolizumab, durvalumab, nivolumab, pembrolizumab, spartalizumab, tremelimumab, and ipilimumab.

7. The method of claim 1, wherein the DR5 agonist comprises a DR5 targeting antibody.

8. The method of claim 7, wherein the DR5 targeting antibody is selected from the group comprising lexatumumab, apomab, AMG655, LByl35, WD-1, KMTR2, and tigatuzumab.

9. The method of claim 1, wherein the composition comprises (a) an effective amount of an inhibitor of a ROCK1 biological activity and/or an effective amount of a checkpoint inhibitor, optionally wherein the checkpoint inhibitor is a CTLA4 inhibitor, a PD-1 inhibitor, a PD-L1 inhibitor, or any combination thereof; and (b) an effective amount of a DR5 agonist in a single composition.

10. A composition comprising:
(a) an effective amount of an inhibitor of a Rho-Associated Coiled-Coil Containing Protein Kinase 1 (ROCK1) biological activity and/or an effective amount of a checkpoint inhibitor, optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor, or any combination thereof; and
(b) an effective amount of a DR5 agonist.

11. The composition of claim 10, wherein the composition comprises a bispecific antibody.

12. The composition of claim 10, further comprising a pharmaceutically acceptable carrier, optionally a pharmaceutically acceptable carrier that is pharmaceutically acceptable for use in a human.

13. A bispecific antibody that binds to a death receptor 5 (DR5) polypeptide and second polypeptide selected from the group consisting of a Rho-Associated Coiled-Coil Containing Protein Kinase 1 (ROCK1) polypeptide, a Cytotoxic T-Lymphocyte Associated Protein 4 (CTLA4) polypeptide, a Programmed Cell Death Protein 1 (PD-1) polypeptide, and a Programmed Death-Ligand 1 (PD-L1) polypeptide, wherein said bispecific antibody comprises a first antigen binding site that is specific for the DR5 polypeptide and a second antigen binding site that is specific for the ROCK1 polypeptide, the CTLA4 polypeptide, the PD-1 polypeptide, or the PD-L1 polypeptide.

14. The bispecific antibody of claim 13, wherein the bispecific antibody comprises a heavy chain variable region and/or a light chain variable as set forth in any of SEQ ID NOs: 1-12.

15. The bispecific antibody of claim 13, wherein the bispecific antibody is humanized.

16. The bispecific antibody of claim 13, further comprising a pharmaceutically acceptable carrier, optionally a pharmaceutically acceptable carrier that is pharmaceutically acceptable for use in a human.

17. A composition for use in treating a tumor and/or a cancer, the composition comprising:
(a) an effective amount of an inhibitor of a Rho-Associated Coiled-Coil Containing Protein Kinase 1 (ROCK1) biological activity and/or an effective amount of a checkpoint inhibitor, optionally wherein the checkpoint inhibitor is a Cytotoxic T-Lymphocyte Associated Protein 4 (CTLA4) inhibitor, a Programmed Cell Death Protein 1 (PD-1) inhibitor, a Programmed Death-Ligand 1 (PD-L1) inhibitor, or any combination thereof; and
(b) an effective amount of a Death Receptor 5 (DR5) agonist.

18. The composition for use of claim 17, wherein the composition comprises a bispecific antibody, and further wherein the bispecific antibody comprises a first antigen binding site that is specific for the DR5 polypeptide and a second antigen binding site that is specific for a ROCK1 polypeptide, a CTLA4 polypeptide, a PD-1 polypeptide, and/or a PD-L1 polypeptide.

19. The composition for use of claim 17, further comprising a pharmaceutically acceptable carrier, optionally a pharmaceutically acceptable carrier that is pharmaceutically acceptable for use in a human.

20. A bispecific antibody for use in treating a tumor and/or a cancer, wherein the bispecific antibody comprises a first binding activity that binds to a death receptor 5 (DR5) polypeptide and a second binding activity that binds to a ROCK1 polypeptide, a CTLA4 polypeptide, a PD-1 polypeptide, and/or a PD-L1 polypeptide.

21. The bispecific antibody for use in treating a tumor and/or a cancer of claim 20, wherein the bispecific antibody comprises a heavy chain variable region and/or a light chain variable as set forth in any of SEQ ID NOs: 1-12.

22. The bispecific antibody for use in treating a tumor and/or a cancer of claim 20, wherein the antibody is humanized.

23. The bispecific antibody for use in treating a tumor and/or a cancer of claim 20, further comprising a pharmaceutically acceptable carrier, optionally a pharmaceutically acceptable carrier that is pharmaceutically acceptable for use in a human.


SEQUENCE LISTING (34 sequences)

SEQ ID NO: 1 - protein, 214 aa, Artificial Sequence
  Artificially synthesized antibody variable region amino acid sequence
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 2 - protein, 451 aa, Artificial Sequence
  Artificially synthesized antibody variable region amino acid sequence
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 213 aa, Artificial Sequence
  Artificially synthesized antibody variable region amino acid sequence
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, Artificial Sequence
  Artificially synthesized antibody variable region amino acid sequence
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 215 aa, Artificial Sequence
  Artificially synthesized antibody variable region amino acid sequence
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 447 aa, Artificial Sequence
  Artificially synthesized antibody variable region amino acid sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSPGK

SEQ ID NO: 7 - protein, 214 aa, Artificial Sequence
  Artificially synthesized antibody variable region amino acid sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 456 aa, Artificial Sequence
  Artificially synthesized antibody variable region amino acid sequence
QVQLVQSGAEMKKPGASVKVSCKTSGYTFTNYKINWVRQAPGQGLEWMGWMNPDTDSTGYPQKFQGRVTMTRNTSISTAYMELSSLRSEDTAVYYCARSYGSGSYYRDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 236 aa, Artificial Sequence
  Artificially synthesized antibody variable region amino acid sequence
MGWSCIILFLVATATGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 465 aa, Artificial Sequence
  Artificially synthesized antibody variable region amino acid sequence
MGWSCIILFLVATATGVHSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 11 - protein, 217 aa, Artificial Sequence
  Artificially synthesized antibody variable region amino acid sequence
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 450 aa, Artificial Sequence
  Artificially synthesized antibody variable region amino acid sequence
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLPGK

SEQ ID NO: 13 - protein, 441 aa, Artificial Sequence
  Artificially synthesized HuDR5 amino acid sequence
MSEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 14 - protein, 413 aa, Artificial Sequence
  Artificially synthesized Chi-DR5 amino acid sequence
MSEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGLWIGLLVPVVLLIGALLVWKTGAWRQWLLCIKRGCERDPESANSVHLSLLDRQTSSTTNDSNHNTEPGKTQKTGKKLLVPVNGNDSADDLKFIFEYCSDIVPFDSWNRLMRQLGLTDNQIQMVKAETLVTREALYQMLLKWRHQTGRSASINHLLDALEAVEERDAMEKIEDYAVKSGRFTYQNAAAQPETGPGGSQCV

SEQ ID NO: 15 - protein, 413 aa, Artificial Sequence
  Artificially synthesized Chi-G4S DR5 amino acid sequence
MEPPGPSTPTASAAARADHYTPGLRPLPKRRLLYSFALLLAVLQAVFVPVTAITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSGGGGSLGLWIGLLVPVVLLIGALLVWKTGAWRQWLLCIKRGCERDPESANSVHLSLLDRQTSSTTNDSNHNTEPGKTQKTGKKLLVPVNGNDSADDLKFIFEYCSDIVPFDSWNRLMRQLGLTDNQIQMVKAETLVTREALYQMLLKWRHQTGRSASINHLLDALEAVEERDAMEKIEDYAVKSGRFTYQNAAAQPETGPGGSQCV

SEQ ID NO: 16 - DNA, 9446 bp, Homo sapiens
  CDS (904)..(4968)
agtctgccccggcggtctccgtttgtttgaacaggaaggcggacatattagtccctctcagcccccctcgccccaccccccaggcattcgccgccgcgactcgccctttccccggctgggaccgcagcccctcccagaagctcccccatcagcagccgccgggacccaactatcgtcttcctcttcgcccgctctccagcctttcctctgctaagtctccatcgggcatcgacctcgccctgccccaccggacaccgtagcagcagccccagcagcgacgggacaaaatgggagagtgaggctgtcctgcgtggaccagctcgtggccgagactgatcggtgcgtcgggccgggccgagtagagccggggacgcggggctagaccgtctacagcgcctctgagcggagcgggcccggcccgtggcccgagcggcggccgcagctggcacagctcctcacccgccctttgctttcgcctttcctcttctccctcccttgttgcccggagggagtctccaccctgcttctctttctctacccgctcctgcccatctcgggacggggacccctccatggcgacggcggccggggcccgctagactgaagcacctcgccggagcgacgaggctggtggcgacggcgctgtcggctgtcgtgaggggctgccgggtgggatgcgactttgggcgtccgagcggctgtgggtcgctgttgcccccggcccggggtctggagagcggaggtcccctcagtgaggggaagacgggggaaccgggcgcacctggtgaccctgaggttccggctcctccgccccgcggctgcgaacccaccgcggaggaagttggttgaaattgctttccgctgctggtgctggtaagagggcattgtcacagcagcagcaacatgtcgactggggacagttttgagactcgatttgaaaaaatggacaacctgctgcgggatcccaaatcggaagtgaattcggattgtttgctggatggattggatgctttggtatatgatttggattttcctgccttaagaaaaaacaaaaatattgacaactttttaagcagatataaagacacaataaataaaatcagagatttacgaatgaaagctgaagattatgaagtagtgaaggtgattggtagaggtgcatttggagaagttcaattggtaaggcataaatccaccaggaaggtatatgctatgaagcttctcagcaaatttgaaatgataaagagatctgattctgcttttttctgggaagaaagggacatcatggcttttgccaacagtccttgggttgttcagcttttttatgcattccaagatgatcgttatctctacatggtgatggaatacatgcctggtggagatcttgtaaacttaatgagcaactatgatgtgcctgaaaaatgggcacgattctatactgcagaagtagttcttgcattggatgcaatccattccatgggttttattcacagagatgtgaagcctgataacatgctgctggataaatctggacatttgaagttagcagattttggtacttgtatgaagatgaataaggaaggcatggtacgatgtgatacagcggttggaacacctgattatatttcccctgaagtattaaaatcccaaggtggtgatggttattatggaagagaatgtgactggtggtcggttggggtatttttatacgaaatgcttgtaggtgatacacctttttatgcagattctttggttggaacttacagtaaaattatgaaccataaaaattcacttacctttcctgatgataatgacatatcaaaagaagcaaaaaaccttatttgtgccttccttactgacagggaagtgaggttagggcgaaatggtgtagaagaaatcaaacgacatctcttcttcaaaaatgaccagtgggcttgggaaacgctccgagacactgtagcaccagttgtacccgatttaagtagtgacattgatactagtaattttgatgacttggaagaagataaaggagaggaagaaacattccctattcctaaagctttcgttggcaatcaactaccttttgtaggatttacatattatagcaatcgtagatacttatcttcagcaaatcctaatgataacagaactagctccaatgcagataaaagcttgcaggaaagtttgcaaaaaacaatctataagctggaagaacagctgcataatgaaatgcagttaaaagatgaaatggagcagaagtgcagaacctcaaacataaaactagacaagataatgaaagaattggatgaagagggaaatcaaagaagaaatctagaatctacagtgtctcagattgagaaggagaaaatgttgctacagcatagaattaatgagtaccaaagaaaagctgaacaggaaaatgagaagagaagaaatgtagaaaatgaagtttctacattaaaggatcagttggaagacttaaagaaagtcagtcagaattcacagcttgctaatgagaagctgtcccagttacaaaagcagctagaagaagccaatgacttacttaggacagaatcggacacagctgtaagattgaggaagagtcacacagagatgagcaagtcaattagtcagttagagtccctgaacagagagttgcaagagagaaatcgaattttagagaattctaagtcacaaacagacaaagattattaccagctgcaagctatattagaagctgaacgaagagacagaggtcatgattctgagatgattggagaccttcaagctcgaattacatctttacaagaggaggtgaagcatctcaaacataatctcgaaaaagtggaaggagaaagaaaagaggctcaagacatgcttaatcactcagaaaaggaaaagaataatttagagatagatttaaactacaaacttaaatcattacaacaacggttagaacaagaggtaaatgaacacaaagtaaccaaagctcgtttaactgacaaacatcaatctattgaagaggcaaagtctgtggcaatgtgtgagatggaaaaaaagctgaaagaagaaagagaagctcgagagaaggctgaaaatcgggttgttcagattgagaaacagtgttccatgctagacgttgatctgaagcaatctcagcagaaactagaacatttgactggaaataaagaaaggatggaggatgaagttaagaatctaaccctgcaactggagcaggaatcaaataagcggctgttgttacaaaatgaattgaagactcaagcatttgaggcagacaatttaaaaggtttagaaaagcagatgaaacaggaaataaatactttattggaagcaaagagattattagaatttgagttagctcagcttacgaaacagtatagaggaaatgaaggacagatgcgggagctacaagatcagcttgaagctgagcaatatttctcgacactttataaaacccaggtaaaggaacttaaagaagaaattgaagaaaaaaacagagaaaatttaaagaaaatacaggaactacaaaatgaaaaagaaactcttgctactcagttggatctagcagaaacaaaagctgagtctgagcagttggcgcgaggccttctggaagaacagtattttgaattgacgcaagaaagcaagaaagctgcttcaagaaatagacaagagattacagataaagatcacactgttagtcggcttgaagaagcaaacagcatgctaaccaaagatattgaaatattaagaagagagaatgaagagctaacagagaaaatgaagaaggcagaggaagaatataaactggagaaggaggaggagatcagtaatcttaaggctgcctttgaaaagaatatcaacactgaacgaacccttaaaacacaggctgttaacaaattggcagaaataatgaatcgaaaagattttaaaattgatagaaagaaagctaatacacaagatttgagaaagaaagaaaaggaaaatcgaaagctgcaactggaactcaaccaagaaagagagaaattcaaccagatggtagtgaaacatcagaaggaactgaatgacatgcaagcgcaattggtagaagaatgtgcacataggaatgagcttcagatgcagttggccagcaaagagagtgatattgagcaattgcgtgctaaacttttggacctctcggattctacaagtgttgctagttttcctagtgctgatgaaactgatggtaacctcccagagtcaagaattgaaggttggctttcagtaccaaatagaggaaatatcaaacgatatggctggaagaaacagtatgttgtggtaagcagcaaaaaaattttgttctataatgacgaacaagataaggagcaatccaatccatctatggtattggacatagataaactgtttcacgttagacctgtaacccaaggagatgtgtatagagctgaaactgaagaaattcctaaaatattccagatactatatgcaaatgaaggtgaatgtagaaaagatgtagagatggaaccagtacaacaagctgaaaaaactaatttccaaaatcacaaaggccatgagtttattcctacactctaccactttcctgccaattgtgatgcctgtgccaaacctctctggcatgtttttaagccaccccctgccctagagtgtcgaagatgccatgttaagtgccacagagatcacttagataagaaagaggacttaatttgtccatgtaaagtaagttatgatgtaacatcagcaagagatatgctgctgttagcatgttctcaggatgaacaaaaaaaatgggtaactcatttagtaaagaaaatccctaagaatccaccatctggttttgttcgtgcttcccctcgaacgctttctacaagatccactgcaaatcagtctttccggaaagtggtcaaaaatacatctggaaaaactagttaaccatgtgactgagtgccctgtggaatcgtgtgggatgctacctgataaaccaggcttctttaaccatgcagagcagacaggctgtttctttgacacaaatatcacaggcttcagggttaagattgctgtttttctgtccttgctttggcacaacacactgagggttttttttattgcgggtttgcctacaggtagattagattaattattactatgtaatgcaagtacagttgggggaaagcttaggtagatatattttttttaaaaggtgctgcctttttggatttataagaaaatgcctgtcagtcgtgatagaacagagttttcctcatatgagtaagaggaagggactttcactttcaagtggaacagccatcactatcaagatcagctcatggaaggagtaaagaaaatatctcaaaatgagacaaactgaagttttgttttttttttaatgacttaagtttttgtgctcttgcaagactatacaaaactattttaagaaagcagtgatatcacttgaacttcagtgccctcactgtagaatttaaaagccttactgttgattgcccatgttggacttgatggagaaattaaatatctttcattatgctttacaaaatactgtatatgtttcagcaagtttggggaatgggagaggacaaaaaaaagttacatttaatctatgcatttttgccaagccatattgagttattttactactagagacattaggaaactaactgtacaaaagaaccaagtttaaaagcattttgtggggtacatcatttctataattgtataatgtatttctttgtggttttaaatgataaagacattaagttaacaaacatataagaaatgtatgcactgtttgaaatgtaaattattcttagaacactttcaatgggggttgcattgtccttttagtgccttaatttgagataattattttactgccatgagtaagtatagaaatttcaaaaaatgtattttcaaaaaattatgtgtgtcagtgagtttttcattgataattggtttaatttaaaatatttagaggtttgttggactttcataaattgagtacaatctttgcatcaaactacctgctacaataatgactttataaaactgcaaaaaatgtagaaggttgcaccaacataaaaaggaaatatggcaatacatccatgatgttttccagttaacataggaattaccagataaatactgttaaactcttgtccagtaacaagagttgattcatatggacagtatgatttattgtttatttttttaaccaaatacctcctcagtaatttataatggctttgcagtaatgtgtatcagataagaagcactggaaaaccgatcgtctctaggatgatatgcatgtttcaagtggtattgaaagccgcactgatggatatgtaataataaacatatctgttattaatatactaatgactctgtgctcatttaatgagaaataaaagtaatttatggatgggtatctttaatttttactgcaatgtgttttctcatggctgaaatgaatggaaaacatacttcaaattagtctctgattgtatataaatgtttgtgaaattccatggttagattaaagtgtatttttaaaagataaaacgtggtcttgggctctaaaacaagagtgggaatagttcacatgaaaagctggctaaagtgtgttggaaaacaattctccatgggtctcttgcatttctacacatcagggaagcagaggctctgacaacctttcttttgactatcttttccaaggatatttatgtagtaaacagccttggaagatagagttctcccttgtgaggatgtcgggttcttttactgtccagtataataaagataacgtctcccacctatgatgcaaacccactgtgtgctcagcacccacctgggccactctgcattgcccacatggggcttggagggcaaagggaactgatggaacgtgaaacttatgctgcttttgctgtgctgtgaatgaagtcatctgactctgacccaggggtctcatctcttttgtcaggatccatgaaactgtgactggcagcatgcaaacaggataaaatctcagcacagttcctgacggtgtccttactaccttctcagactcatcaccattacctaatattttaaggtccagttgcagagaacagagttaattctaattagtttaagcaagagaagactttccaaatgacatactaatctgaacttagaattgttgagaactgagagagctttgaggaatcactcccaatgccacattacagaactgggtcaaccaggagagctgcttcctcttctaagttcaggaaaccactcactcccaagtataccaatattgagagggtccagatagtgccataatcaggaatccacactaccactgctggttctctgcaacacttgtcaggcttgctacaatctacctccagttagatgttttgtaccattcctcttgccacttaagaacctcatgtctgcacactgcctcagaaaaggcacttccatgaccctgcttgctggcatttagcacagcctgcatctcacgtctgccttctctaggtgcatctacgtggccaaaattaaatcacatccagacccctagctgcaagaaagtctgagaaatatagcctctagctttctctctaatttgttacaggaggacacactaggagtgtggaatgaaggttcagcattgagttagcccatgcacccacacctgtaagcttctctgagatcaggaactccagctttgggcaacaaagaaaacatagccactgtcattattcacaggttcactcatgacctgtcatttggggtttcagcttaaattaacccattcacccttcatttaaagtaaaaaaaataagtaaaagaacttaaatgacaaactcatagttacttgagtttgtaattcatagttaactttcacagttctataatcaatctggaattcagagtgaggtagagatcaagcagtatttctcccatatggatattcagttgatactgcagtattcattgaaaatacttctttcctgcatcgtaattcaggtgaacaatccattggtgttatttctgtgtccttgccaatatcacacttttaattactgtaactttataataagattttatatctgtagtagaagtccaactttgtaatccctcccaattggcttggctattcttggccttttgcatttccatataattttttatttttgttttgaaacgaagtttcgctcttgttgcccaggctggagtgcagtggcgcgatctcggctcactgcaacctccgcctcccaggttcaagtgatcctcctgcctcagactcccgagtagctgggattacaggtgcccaccaccatgcccagctaatttttgtatttttagtagagacagggtttcaccatgttggccagcctggtcttgaactcctgacctcaggtgatccacacccaccttggcctcccaaagtgctgggattacaggcatgagccaccgcgcctggttgcattcccatataaatttaaaaacaatctactgaaagatgtatacgacctccaccaagaaaattgtaagacatttggagaagtttaaaaagacatgaataaacggaaatggtttttttaaataaacaaatgatttttaaagttgatcattaggttaataaatagtcatataatgacaatccgatgagattatgttgcccagggtggtctctaactcctagcctcaagcagttttactctctcattacaggtgtaggccaccgtgcctggcctgacacttggaaatcttaatcacaattattctttgtttttataatgtttactgttcaaatcaatttctggttggatagtatgtatatataaagccatgctcaatggcctagatatatttactgccaaatcttttatgcttttagtgttttttttctggacaagatctatgcttattgcaataaattaaatgcaaagaatattctagtagaaagtcagtctgcacttcctaaaccccaccccaccccaccaattccttcataactgttattaacagttagctgtgttttctaatggattatctccaaagtatatgtaactcagatttttgtgaggtaggaaaagaataacattaattccacttcacatattgaaagtctaacatacaaagagattaaatatctcctttcttttgccaaatgttaatgaattgtcttatgagaattaagatatgatactttctcccaagctatactgtttcccataaagtatttgagatgttggagattcaagggaaaaaatataaatgcttagaaataaagtgtataatgcaaagattgtgaataaagtgacacttgtatgttaatcctataaattggtttcttgtaccatgtaggatggtttggctattaaattgaattaagtggttca

SEQ ID NO: 17 - protein, 1354 aa, Homo sapiens
MSTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNRTSSNADKSLQESLQKTIYKLEEQLHNEMQLKDEMEQKCRTSNIKLDKIMKELDEEGNQRRNLESTVSQIEKEKMLLQHRINEYQRKAEQENEKRRNVENEVSTLKDQLEDLKKVSQNSQLANEKLSQLQKQLEEANDLLRTESDTAVRLRKSHTEMSKSISQLESLNRELQERNRILENSKSQTDKDYYQLQAILEAERRDRGHDSEMIGDLQARITSLQEEVKHLKHNLEKVEGERKEAQDMLNHSEKEKNNLEIDLNYKLKSLQQRLEQEVNEHKVTKARLTDKHQSIEEAKSVAMCEMEKKLKEEREAREKAENRVVQIEKQCSMLDVDLKQSQQKLEHLTGNKERMEDEVKNLTLQLEQESNKRLLLQNELKTQAFEADNLKGLEKQMKQEINTLLEAKRLLEFELAQLTKQYRGNEGQMRELQDQLEAEQYFSTLYKTQVKELKEEIEEKNRENLKKIQELQNEKETLATQLDLAETKAESEQLARGLLEEQYFELTQESKKAASRNRQEITDKDHTVSRLEEANSMLTKDIEILRRENEELTEKMKKAEEEYKLEKEEEISNLKAAFEKNINTERTLKTQAVNKLAEIMNRKDFKIDRKKANTQDLRKKEKENRKLQLELNQEREKFNQMVVKHQKELNDMQAQLVEECAHRNELQMQLASKESDIEQLRAKLLDLSDSTSVASFPSADETDGNLPESRIEGWLSVPNRGNIKRYGWKKQYVVVSSKKILFYNDEQDKEQSNPSMVLDIDKLFHVRPVTQGDVYRAETEEIPKIFQILYANEGECRKDVEMEPVQQAEKTNFQNHKGHEFIPTLYHFPANCDACAKPLWHVFKPPPALECRRCHVKCHRDHLDKKEDLICPCKVSYDVTSARDMLLLACSQDEQKKWVTHLVKKIPKNPPSGFVRASPRTLSTRSTANQSFRKVVKNTSGKTS

SEQ ID NO: 18 - DNA, 1997 bp, Homo sapiens
  CDS (173)..(844)
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 19 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 20 - DNA, 1887 bp, Homo sapiens
  CDS (173)..(697)
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 21 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 22 - DNA, 2097 bp, Homo sapiens
  CDS (57)..(923)
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 23 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 24 - DNA, 868 bp, Homo sapiens
  CDS (70)..(807)
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctagcatgatgtctgcctatcatagtcattcagtgattgttgaataaatgaatgaatgaataaca

SEQ ID NO: 25 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 26 - DNA, 3292 bp, Homo sapiens
  CDS (70)..(600)
agttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 27 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 28 - DNA, 3634 bp, Homo sapiens
  CDS (70)..(942)
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 29 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 30 - DNA, 3998 bp, Homo sapiens
  CDS (138)..(1460)
agcctggacacataaatcagcacgcggccggagaaccccgcaatctctgcgcccacaaaatacaccgacgatgcccgatctactttaagggctgaaacccacgggcctgagagactataagagcgttccctaccgccatggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagtcccagctgtggaggagacggtgacctccagcccagggactcctgcctctccctgttctctctcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaagtgtgattctcttcaggaagtcagaccttccctggtttaccttttttctggaaaaagcccaactggactccagtcagtaggaaagtgccacaattgtcacatgaccggtactggaagaaactctcccatccaacatcacccagtggatggaacatcctgtaacttttcactgcacttggcattatttttataagctgaatgtgataataaggacactatggaaatgtctggatcattccgtttgtgcgtactttgagatttggtttgggatgtcattgttttcacagcacttttttatcctaatgtaaatgctttatttatttatttgggctacattgtaagatccatctacacagtcgttgtccgacttcacttgatactatatgatatgaaccttttttgggtggggggtgcggggcagttcactctgtctcccaggctggagtgcaatggtgcaatcttggctcactatagccttgacctctcaggctcaagcgattctcccacctcagccatccaaatagctgggaccacaggtgtgcaccaccacgcccggctaattttttgtattttgtctagatataggggctctctatgttgctcagggtggtctcgaattcctggactcaagcagtctgcccacctcagactcccaaagcggtggaattagaggcgtgagcccccatgcttggccttacctttctacttttataattctgtatgttattattttatgaacatgaagaaactttagtaaatgtacttgtttacatagttatgtgaatagattagataaacataaaaggaggagacatacaatgggggaagaagaagaagtcccctgtaagatgtcactgtctgggttccagccctccctcagatgtactttggcttcaatgattggcaacttctacaggggccagtcttttgaactggacaaccttacaagtatatgagtattatttataggtagttgtttacatatgagtcgggaccaaagagaactggatccacgtgaagtcctgtgtgtggctggtccctacctgggcagtctcatttgcacccatagcccccatctatggacaggctgggacagaggcagatgggttagatcacacataacaatagggtctatgtcatatcccaagtgaacttgagccctgtttgggctcaggagatagaagacaaaatctgtctcccacgtctgccatggcatcaagggggaagagtagatggtgcttgagaatggtgtgaaatggttgccatctcaggagtagatggcccggctcacttctggttatctgtcaccctgagcccatgagctgccttttagggtacagattgcctacttgaggaccttggccgctctgtaagcatctgactcatctcagaaatgtcaattcttaaacactgtggcaacaggacctagaatggctgacgcattaaggttttcttcttgtgtcctgttctattattgttttaagacctcagtaaccatttcagcctctttccagcaaacccttctccatagtatttcagtcatggaaggatcatttatgcaggtagtcattccaggagtttttggtcttttctgtctcaaggcattgtgtgttttgttccgggactggtttgggtgggacaaagttagaattgcctgaagatcacacattcagactgttgtgtctgtggagttttaggagtggggggtgacctttctggtctttgcacttccatcctctcccacttccatctggcatcccacgcgttgtcccctgcacttctggaaggcacagggtgctgctgcctcctggtctttgcctttgctgggccttctgtgcaggacgctcagcctcagggctcagaaggtgccagtccggtcccaggtcccttgtcccttccacagaggccttcctagaagatgcatctagagtgtcagccttatcagtgtttaagatttttcttttatttttaatttttttgagacagaatctcactctctcgcccaggctggagtgcaacggtacgatcttggctcagtgcaacctccgcctcctgggttcaagcgattctcgtgcctcagcctccggagtagctgggattgcaggcacccgccaccacgcctggctaatttttgtatttttagtagagacggggtttcaccatgttggtcaggctggtctcgaactcctgacctcaggtgatccaccttggcctccgaaagtgctgggattacaggcgtgagccaccagccaggccaagctattcttttaaagtaagcttcctgacgacatgaaataattgggggttttgttgtttagttacattaggctttgctatatccccaggccaaatagcatgtgacacaggacagccatagtatagtgtgtcactcgtggttggtgtcctttcatgcttctgccctgtcaaaggtccctatttgaaatgtgttataatacaaacaaggaagcacattgtgtacaaaatacttatgtatttatgaatccatgaccaaattaaatatgaaaccttatataaaaa

SEQ ID NO: 31 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 32 - DNA, 3911 bp, Homo sapiens
  CDS (138)..(1373)
agcctggacacataaatcagcacgcggccggagaaccccgcaatctctgcgcccacaaaatacaccgacgatgcccgatctactttaagggctgaaacccacgggcctgagagactataagagcgttccctaccgccatggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaagtgtgattctcttcaggaagtcagaccttccctggtttaccttttttctggaaaaagcccaactggactccagtcagtaggaaagtgccacaattgtcacatgaccggtactggaagaaactctcccatccaacatcacccagtggatggaacatcctgtaacttttcactgcacttggcattatttttataagctgaatgtgataataaggacactatggaaatgtctggatcattccgtttgtgcgtactttgagatttggtttgggatgtcattgttttcacagcacttttttatcctaatgtaaatgctttatttatttatttgggctacattgtaagatccatctacacagtcgttgtccgacttcacttgatactatatgatatgaaccttttttgggtggggggtgcggggcagttcactctgtctcccaggctggagtgcaatggtgcaatcttggctcactatagccttgacctctcaggctcaagcgattctcccacctcagccatccaaatagctgggaccacaggtgtgcaccaccacgcccggctaattttttgtattttgtctagatataggggctctctatgttgctcagggtggtctcgaattcctggactcaagcagtctgcccacctcagactcccaaagcggtggaattagaggcgtgagcccccatgcttggccttacctttctacttttataattctgtatgttattattttatgaacatgaagaaactttagtaaatgtacttgtttacatagttatgtgaatagattagataaacataaaaggaggagacatacaatgggggaagaagaagaagtcccctgtaagatgtcactgtctgggttccagccctccctcagatgtactttggcttcaatgattggcaacttctacaggggccagtcttttgaactggacaaccttacaagtatatgagtattatttataggtagttgtttacatatgagtcgggaccaaagagaactggatccacgtgaagtcctgtgtgtggctggtccctacctgggcagtctcatttgcacccatagcccccatctatggacaggctgggacagaggcagatgggttagatcacacataacaatagggtctatgtcatatcccaagtgaacttgagccctgtttgggctcaggagatagaagacaaaatctgtctcccacgtctgccatggcatcaagggggaagagtagatggtgcttgagaatggtgtgaaatggttgccatctcaggagtagatggcccggctcacttctggttatctgtcaccctgagcccatgagctgccttttagggtacagattgcctacttgaggaccttggccgctctgtaagcatctgactcatctcagaaatgtcaattcttaaacactgtggcaacaggacctagaatggctgacgcattaaggttttcttcttgtgtcctgttctattattgttttaagacctcagtaaccatttcagcctctttccagcaaacccttctccatagtatttcagtcatggaaggatcatttatgcaggtagtcattccaggagtttttggtcttttctgtctcaaggcattgtgtgttttgttccgggactggtttgggtgggacaaagttagaattgcctgaagatcacacattcagactgttgtgtctgtggagttttaggagtggggggtgacctttctggtctttgcacttccatcctctcccacttccatctggcatcccacgcgttgtcccctgcacttctggaaggcacagggtgctgctgcctcctggtctttgcctttgctgggccttctgtgcaggacgctcagcctcagggctcagaaggtgccagtccggtcccaggtcccttgtcccttccacagaggccttcctagaagatgcatctagagtgtcagccttatcagtgtttaagatttttcttttatttttaatttttttgagacagaatctcactctctcgcccaggctggagtgcaacggtacgatcttggctcagtgcaacctccgcctcctgggttcaagcgattctcgtgcctcagcctccggagtagctgggattgcaggcacccgccaccacgcctggctaatttttgtatttttagtagagacggggtttcaccatgttggtcaggctggtctcgaactcctgacctcaggtgatccaccttggcctccgaaagtgctgggattacaggcgtgagccaccagccaggccaagctattcttttaaagtaagcttcctgacgacatgaaataattgggggttttgttgtttagttacattaggctttgctatatccccaggccaaatagcatgtgacacaggacagccatagtatagtgtgtcactcgtggttggtgtcctttcatgcttctgccctgtcaaaggtccctatttgaaatgtgttataatacaaacaaggaagcacattgtgtacaaaatacttatgtatttatgaatccatgaccaaattaaatatgaaaccttatataaaaa

SEQ ID NO: 33 - protein, 411 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 34 - protein, 5 aa, Artificial Sequence
  Artificially synthesized peptide linker
GGGGS